(19) United States
(12) Patent Application Publication
(10) Pub. No.: US 2025/0367166 A1
(43) Pub. Date: Dec. 4, 2025

(54) NEXT GENERATION DIPROVOCIMS THAT ACTIVATE THE INNATE AND ADAPTIVE IMMUNE RESPONSE

(71) Applicants: The Scripps Research Institute, La Jolla, CA (US); THE BOARD OF REGENTS OF THE UNIVERSITY OF TEXAS SYSTEM, Austin, TX (US)

(72) Inventors: Dale L. Boger, La Jolla, CA (US); Bruce Beutler, Irving, TX (US)

(21) Appl. No.: 18/859,673

(22) PCT Filed: Apr. 27, 2023
(86) PCT No.: PCT/US23/66308

§ 371 Date: Oct. 24, 2024

Related U.S. Application Data
(60) Provisional application No. 63/363,848, filed on Apr. 29, 2022.

(51) Int. Cl. A61K 31/4025; A61P 35/00; C07D 207/16

(52) U.S. Cl. CPC A61K 31/4025; A61P 35/00; C07D 207/16

(57) ABSTRACT

The present invention contemplates new members of the diprovocim family of compounds that exhibit improvements in both potency and efficacy in the murine system, permitting more effective use in vivo in animal models while maintaining the remarkable activity agonist towards human TLR1/TLR2. The prototypical new agonist called diprovocim-X exhibits the same excellent potency and efficacy of diprovocim-1 in human THP-1 cells (EC₅₀ of 140 pM vs 110 pM with efficacy 100% that of diprovocim-1 and Pam3CSK4), and displays a superb EC₅₀ of 750 pM in mouse macrophages with an efficacy 550% that of diprovocim-1. Diprovocim-X served as an adjuvant in vivo in mice when co-administrated with a non-immunogenic antigen (OVA), indicating stimulation of the adaptive immune response. These the new diprovocim family compounds are now functionalized for linkage to antigenic, targeting, or delivery moieties, properties to enable precision activation of coordinated innate and adaptive immune responses in target tissues.

### CROSS-REFERENCE TO RELATED APPLICATION

This application claims priority to U.S. application Ser. No. 63/363,848, filed on Apr. 29, 2022, whose disclosures are incorporated herein by reference.

### GOVERNMENTAL SUPPORT

This invention was made with governmental support under AI082657, CA042056 and AI100627 awarded by the National Institutes of Health. The government has certain rights in the invention.

### REFERENCE TO SEQUENCE LISTING

The instant application contains a Sequence Listing that has been submitted electronically in in .XML format and is hereby incorporated by reference in its entirety. The Sequence Listing: is named “9709-298.xml”, is 2,099 bytes in size, and was created on Apr. 26, 2023.

### BACKGROUND ART

Controlled manipulation of both the inflammatory innate immune response and ensuing adaptive immune response is necessary for precision therapeutics targeting infectious diseases and cancer while avoiding toxic side effects. Immunostimulatory small molecules with defined specificities that activate both innate and adaptive immune responses are therefore highly desirable but are still quite rare, and even more so those with comprehensively characterized molecular and cellular mechanisms. Most are mimics or modifications of antigenic microbial or viral components that act as ligands for innate immune receptors (e.g.; LPS, lipopeptides, nucleic acids); as such their actions are separated, either or both spatially and temporally, from adaptive immune activation by co-administered antigens. Furthermore, they represent structurally unattractive starting points for drug discovery, being difficult to structurally modify and chemically prepare. However, the endogenous protein targets on which they act and the intricate atomic-level mechanisms they activate provide powerful opportunities for the development of new therapeutics.^{1,2}

The Toll-like receptors (TLRs) comprise one family of innate immune receptors, collectively mediate the recognition of most microbes, and elicit intracellular signaling leading to the release of inflammatory cytokines and chemokines that provide critical activating signals to adaptive immune cells. TLR agonists are attractive adjuvants for use in prophylactic vaccination against either bacterial or viral pathogens,^{3,4} and as immunostimulators in the field of cancer immunotherapy where the adaptive immune system is exploited to not only identify and eradicate cancer cells based on their expression of neo-antigens but also to form a long lasting systemic anti-tumor memory response (antigen-specific anti-tumor immunity).⁵

Studies^{6,7} conducted over 30 years ago discovered the immune-activating N-terminal segments of bacterial lipoproteins and lipopeptides that were later shown to act by heterodimerization of TLR1/TLR2.^{8,9} Such agonists based on the lipoproteins are effective vaccine adjuvants^{6,7} and continue to be widely studied today.^{10,11}

TLR2 requires heterodimerization with either TLR1 or TLR6 for activation. Bacterial triacylated proteins or peptides activate TLR1/TLR2 (e.g.; Pam3CSK4^{6,7}) whereas diacylated lipopolypeptides stimulate TLR2/TLR6 (e.g.; MALP-2^{9,10}). Complementary to recent studies that disclosed the only other known and rare small molecule TLR2/TLR1 agonists,^{10,12} and through screening a compound library designed to promote cell surface receptor dimerization,¹³ we discovered and characterized the diprovocims as a new and especially potent class of synthetic small molecule TLR2/TLR1 agonists that bear no structural similarity to any other natural or synthetic TLR agonist.¹⁴⁻¹⁶

The most potent diprovocims elicit full agonist activity at extraordinarily low concentrations (EC₅₀=110 pM) in human cells, being more potent than any known TLR agonist. Moreover, the efficacy of the class matches that of naturally-derived TLR agonists such as Pam3CSK4 or other lipopeptides but are even more potent. A representative of the series, compound 1 (diprovocim-1, below), was shown to act as an effective adjuvant

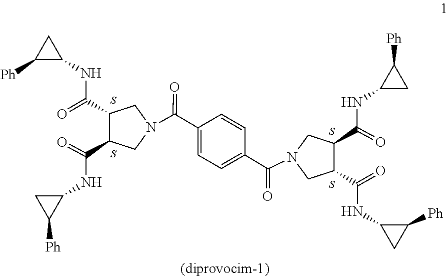

in mice when co-administered by conventional intramuscular (i.m.) injection (vaccination) with the antigen ovalbumin (OVA), which is non-immunogenic in the unadjuvanted state.¹⁴ It was further shown to act synergistically with a checkpoint inhibitor (anti-PD-L1), where the combination treatment eradicated tumors in mice implanted with an immunogen bearing murine melanoma (B16-OVA) and protected mice from tumor rechallenge.¹⁴ This impressive in vivo activity was observed with diprovocim/OVA co-administration i.m., rather than with intratumor adjuvant administration that has been a convention in recent studies.

Herein, we detail studies that led to the identification of a site and manner by which the diprovocims could be functionalized, permitting covalent linkage to candidate protein and peptide antigens or coupling with a targeting or delivery moiety,¹⁷ without impacting TLR1/TLR2 activity. Because the potency and efficacy of the diprovocims on human receptors are superb, whereas their activity is significantly weaker in murine systems typically employed in initial in vivo studies, a second and equally important advance disclosed herein is the identification of substantial improvements in both potency and efficacy on mTLR1/TLR2 while maintaining full activity on hTLR1/TLR2.

### BRIEF SUMMARY OF THE INVENTION

The present invention contemplates a compound of Formula I, below, wherein R¹, R² and R³

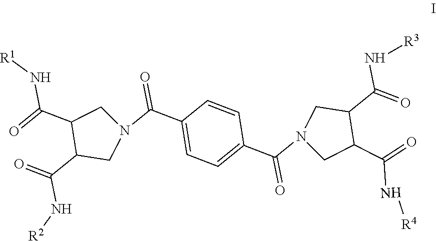

are the same or different and are a trans-2-phenylcyclopropyl, or a trans-2-(4-fluorophenyl)-cyclopropyl group. R⁴ is a composite of (a) a hydrocarbyl group bonded to the depicted amido nitrogen atom and (b) a substituent group bonded to the hydrocarbyl group as discussed below. The hydrocarbyl group has whose longest chain of atoms has a total length that is about that of a saturated chain of about 2 carbon atoms (an ethylene group), and a length that is less than that of a saturated chain of about 20 carbon atoms [an eicosylene group]. That hydrocarbyl group includes a methylene group (—CH₂—) that is 1) bonded directly to or 2) bonded indirectly and distal to the amido nitrogen of the depicted —C(O)NH—R⁴ group. The methylene group is also bonded to a substituent group that is one or another of i) a phenyl group that includes a substituent selected from the group consisting of a hydroxyl, amino, carboxy, C₁-C₆ alkyl carboxylate, sulfo, C₁-C₆ alkyl sulfonate, fluoro, azido and an ethynyl group, ii) a hydroxyl, a mercaptan, amine, mono- or disubstituted amine, an azido group or iii) to an oxygen, nitrogen or sulfur atom that is part of a substituent group that is selected from the group consisting of an ether, ester, carbonate, carbamate, thioether, thioester, thiourea, amido, mono- or disubstituted amide, urea, and a N′-mono or N′,N′-disubstituted urea. The substituent group itself contains a chain of atoms that include zero to four oxygens, zero to two sulfur atoms, and zero to four nitrogen atoms, with the proviso that the sum of the sulfur, nitrogen and oxygen atoms in the substituent is at least one, and not greater than eight. The length of the hydrocarbyl group together with the substituent bonded to the methylene of that hydrocarbyl group is less than that of a saturated chain of about 24 carbon atoms [a tetracosylene group; (—C₂₄H₄₈—)].

In a contemplated compound, each depicted pyrrolidinyldicarboxamido group has the (S,S) configuration. The bonds to the cyclopropyl moiety have a (1S,2R) configuration. Preferably, a contemplated compound has the structural Formula Ia, and the R¹⁻⁴ moieties are as described above

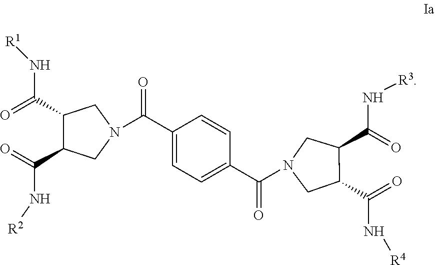

A preferred compound is also a single enantiomer.

A pharmaceutical composition that comprises a concentration of a contemplated compound of Formula I that is effective to induce release of TNF-α from one or both of in vitro cultured human PMA differentiated THP-1 cells and/or mouse macrophages. The compound is present dissolved or dispersed in a physiologically tolerable diluent in that pharmaceutical composition. The compound of that pharmaceutical composition is a single enantiomer.

A method of enhancing an immunogen-specific IgG humoral immune response titer of an immunized mammalian subject is also contemplated. That method comprises contacting immune cells of that mammalian subject with composition containing an adjuvant-effective amount of a compound of Formula I and an immunogen to which the response is to be enhanced that are dissolved or dispersed in a physiologically tolerable diluent. A contemplated method provides an enhancement in IgG titer that is about 2 to about 4 times the titer observed due contacting immune cells with the same amount of immunogen dissolved or dispersed in a physiologically tolerable diluent in the absence of the compound of Formula I, when the titers are measured 14 days after immune cell contacting.

The contacted immune cells are preferably those of a human subject, and that contact is preferably carried out in vivo. It is also preferred that the compound of Formula I be a single enantiomer.

### BRIEF DESCRIPTION OF THE DRAWINGS

In the drawings forming a portion of this disclosure

FIG. 1 is a timeline for in vivo assaying of diprovocim-X adjuvant activity in which mice were immunized i.m. with saline, ovalbumin (OVA), diprovocim-1+OVA, or diprovocim-X+OVA; diprovocims and OVA were administered at 200 μg and 100 μg per mouse, respectively. Immunizations (#1, initial; #2, boost) are indicated. Assays are indicated in gray ovals.

FIG. 2 in two parts as FIG. 2A and FIG. 2B are graphs that illustrate cytotoxic T lymphocyte (CTL) assay results (FIG. 2A) in which CD8 T cell cytotoxic activity towards target cells [SIINFEKL-loadedC57BL/6J (SEQ ID NO: 1) splenocytes¹⁴] were measured 9 days after initial immunization (open circles) and 9 days after a boost (filled circles). Data for OVA administered alone are shown as squares, data for Diprovocim-1+OVA are shown as triangles, and data for Diprovocim-X+OVA are show as diamonds, with open and closed symbols being as timed for saline. Data points represent individual mice. FIG. 2B shows B16-OVA tumor volume was measured on the indicated days after B16-OVA administration (N=8, 8, 8, 5 mice respectively for saline, OVA, diprovocim-1+OVA, diprovocim-X+OVA). P values represent significance of difference on day 22 compared to saline. Mean±SD are plotted. P values were determined by one-way ANOVA and post-hoc Tukey multiple comparisons test. *P<0.05; ****P<0.0001; ns, not significant;

FIG. 3 in three parts as the graphs of FIGS. 3A, 3B and 3C that illustrate serum OVA-specific IgG (FIG. 3A), IgG1 (FIG. 3B), and IgG2b (FIG. 3C) concentrations measured by ELISA 14 days after initial immunization, and in which data points represent individual mice. Circles represent data for saline control, squares represent data for OVA control, triangles represent data for diprovocim-1+OVA, and diamonds represent data for diprovocim-X+OVA. Mean±SD are plotted and P values were determined by one-way ANOVA and post-hoc Tukey multiple comparisons test; *P=0.0502, ****P<0.0001, and “NS”=not significant;

FIG. 4 illustrates Reaction Scheme 1 that shows the reactions leading to Compound S6;

FIG. 5 illustrates Reaction Scheme 2 that shows the reactions leading to Compound S8;

FIG. 6 illustrates Reaction Scheme 3 that shows the reactions leading to Compound S10;

FIG. 7 illustrates Reaction Scheme 4 that shows the reactions leading to Compounds S13 and S15;

FIG. 8 illustrates Reaction Scheme 5 that shows the reactions leading to Compound S19;

FIG. 9 illustrates Reaction Scheme 6 that shows the reactions leading to Compound S21; and

FIG. 10 illustrates Reaction Scheme 7 that shows the reactions leading to Compounds S26 and S27.

### DEFINITIONS

Antibody: a polypeptide that immunologically binds to a ligand group. Antibodies, as used herein, are immunoglobulin molecules and immunologically active fragments of immunoglobulin molecules. Such portions known in the art as Fab, Fab′; F(ab′)₂ and F_{V} are included. Typically, antibodies bind ligands that range in size from about 6 through about 34 Angstroms (Å) with association constants in the range of about 104 to about 10¹⁰ M⁻¹, and as high as 10¹³ M⁻¹. Antibodies can bind a wide range of ligands, including small molecules such as steroids and prostaglandins, biopolymers such as nucleic acids, proteins and polysaccharides, and synthetic polymers such as polypropylene.

An “antibody combining site” or “paratope” is that structural portion of an antibody molecule comprised of a heavy and light chain variable and hypervariable regions that specifically binds to (immunoreacts with) an “antigen” or “epitope”.

The term “antibody” is meant to particularly encompass monoclonal antibodies that are suitable for injection (pharmaceutically acceptable) into a diseased mammal in need of treatment without undo adverse effects due to contaminants. Such monoclonal antibodies can be obtained from the animal species that is immunized as discussed herein, such as a human. Or, the antibodies can be induced in one animal and the antibody-producing cells modified to produce antibody protein sequences of the mammal to be immunized. Although other species of mammal are contemplated for immunization, a human is a particularly preferred recipient of the immunization. As a consequence, a contemplated monoclonal antibody that was originally induced in a mouse, for example, can be more useful to a human recipient as a so-called “humanized” antibody, or as a “chimeric” antibody. These terms are used herein as described in International Nonproprietary Names (INN) for biological and biotechnological substances (a review), World Health Organization (2016), § 2.7.

The word “antigen” has been used historically to designate an entity that is bound by an antibody or receptor, and also to designate the entity that induces the production of the antibody. More current usage limits the meaning of antigen to that entity bound by an antibody or receptor, whereas the word “immunogen” is used for the entity that induces antibody production or binds to the receptor. Where an entity discussed herein is both immunogenic and antigenic, reference to it as either an immunogen or antigen is typically made according to its intended utility.

The term “immunoreact” in its various forms is used herein to refer to specific binding between an antigenic determinant-containing molecule (antigen) and a molecule containing an antibody combining site such as a whole antibody molecule or a paratope-containing portion thereof.

An “antigenic determinant” is the structural portion of the antigen that is immunologically bound by an antibody combining site or T cell receptor. The term is also used interchangeably with “epitope”. Antibodies can bind a single epitope of an antigen (monoclonal) or multiple epitopes (polyclonal). In a proteinaceous material, the length of a linear epitope is usually recited as being about 5 to about 7 amino acid residues.

The articles “a” and “an” are used herein to refer to one or to more than one (i.e., to at least one) of the grammatical object of the article. By way of example, “an element” means one element or more than one element.

The word “hydrocarbyl” is used herein as a short-hand term for a non-aromatic group that includes straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrocarbyl groups, are referred to herein as portions of substituent groups or radicals, as discussed hereinafter.

Where a specific aliphatic hydrocarbyl substituent group is intended, that group is recited; i.e., methyl, ethyl, butyl, tert-butyl, hexyl, hexenyl, 2-ethylhexyl, dodecyl (C₁₂), octadecyl (C₁₈). A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Although long chain (e.g., C₁₈) hydrocarbyl groups are contemplated, examples of shorter (C₁-C₄) groups are used illustratively here. Such illustrative alkyl radicals include ethyl, n-propyl, isopropyl, n-butyl, isobutyl, sec-butyl, tert-butyl and cyclopropyl. Examples of suitable alkenyl radicals include ethenyl (vinyl), 2-propenyl, 3-propenyl, 1,4-butadienyl, 1-butenyl, 2-butenyl, and 3-butenyl. Examples of alkynyl radicals include ethynyl, 2-propynyl, 1-propynyl, 1-butynyl, 2-butynyl, 3-butynyl, and 1-methyl-2-propynyl.

As a skilled worker will understand, a substituent that cannot exist such as a C₁ alkenyl group is not intended to be encompassed by the word “hydrocarbyl” in that an alkenyl group must have two carbon atoms, although such substituents with two or more carbon atoms are intended.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, n-propoxy, isopropoxy, allyloxy, n-butoxy, iso-butoxy, sec-butoxy, and tert-butoxy groups.

A “ligand” a molecule or portion thereof having a structural region that binds specifically to a particular receptor molecule, usually via electrostatic forces and/or hydrogen bonds. An exemplary ligand is the epidermal growth factor molecule.

A “marker molecule” (antigen or immunogen) can be but need not be expressed on the cell surface, and rather can be expressed anywhere in the diseased cell. The reason for that is that substantially all of the native cellular proteins of mammals are processed into shorter peptides by the cell and bound extracellularly by class I MHC molecules. Such native proteins are typically so processed during the organism's immaturity, and T cells or other immune cells that may be induced by those native protein portions are eliminated by the organism prior to maturity, resulting in “self protein” tolerance. As a result, except in cases of certain immunological diseases, only foreign peptides or neoantigenic peptides caused by disease such as cancer whose cells result from mutation are recognized as “foreign” and induce an immune response to the MHC-bound peptide.

Further, as to mutations, at times the mutation can be a frame-shift mutation, and an un-natural sequence of amino acids results. The marker molecule can also be a “tumor antigen;” that is, a protein that can be expressed by other cells during embryonic development, for example, but is characteristically expressed much more by tumors than by normal cells. Or the marker molecule can be an oncogene product, for example, an abnormal fusion protein created by a recombination event within tumor cells. The marker molecule can also be the product of an infectious agent such as a virus or bacterium as well.

A “peptide/polypeptide” is an oligomer or polymer comprising at least two amino acid residues in which adjacent residues are linked by a peptide bond between the alpha-amino group of one residue and the alpha-carboxyl group of an adjacent residue. The primary structure of a polypeptide has a primary amine group at one terminus and a carboxylic acid group at the other terminus of the polymer. A peptide or polypeptide is depicted herein and usually in the art from left to right and in the direction from amino-terminus to carboxy-terminus. Also, a polypeptide in aqueous solution is usually in one or more zwitterionic forms depending on the pH value of the solution. The words “peptide” and “polypeptide” are used interchangeably herein.

A “protein” is a single polypeptide or set of cross-linked polypeptides comprising more than about 100 amino acid residues. Proteins can have chemical cross-linking, e.g., via disulfide bridges, within the same polypeptide chain or between adjacent polypeptides. When a protein is glycosylated it can be called a glycoprotein. When a protein comprises one or more discrete polypeptide/protein subunits linked together, as by a peptide linkage, amino acid residue sequence, disulfide bridge, and the like, the protein is frequently termed a fusion protein, fusion polypeptide, chimeric fusion, and the like.

A “receptor” is a biologically active proteinaceous molecule having a structural region that specifically binds to (or with) another molecule (ligand). An exemplary receptor molecule is an antibody combining site or a transmembrane cellular protein molecule involved in intra- or intercellular signaling such as the endothelial growth hormone receptor referred to as EGFR, ERBB and also as HER2, and the like.

The term “residue” is used interchangeably with the phrase amino acid residue. All amino acid residues identified herein are in the natural or L-configuration, unless otherwise described. In keeping with standard polypeptide nomenclature, [IUPAC-IUB Tentative Rules, J. Biol. Chem., 243:3557-3559 (1968)], abbreviations for amino acid residues are as shown in the following Table of Correspondence.

TABLE OF CORRESPONDENCE

| 1-Letter | 3-Letter | AMINO ACID NAME |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| Z | Glx | L-glutamic acid |
| | | or |
| | | L-glutamine |
| W | Trp | L-tryptophan |
| R | Arg | L-arginine |
| D | Asp | L-aspartic acid |
| N | Asn | L-asparagine |
| B | Asx | L-aspartic acid |
| | | or |
| | | L-asparagine |
| C | Cys | L-cysteine |

Abbreviations Used
Abu, aminobutyric acid; DME, dimethoxyethane; DMF, dimethylformamide; DMSO, dimethylsulfoxide; DMAP, 4-(dimethylamino) pyridine; EDCI, 1-ethyl-3-(3-(dimethylamino)propyl) carbodiimide; HOAt, 1-hydroxy-7-azabenzotriazole; HOBt, 1-hydroxybenzotriazole; IFN, interferon; IL, interleukin; LPS, lipopolysaccharide; Met, methionine NF-κB, nuclear factor kappa-light-chain-enhancer of activated B cells; OVA, ovalbumin; rt, room temperature; TLR, Toll-like receptor; TNF-α, tumor necrosis factor alpha; min, minute; h, hour; ca., about; Boc or tBoc, tertiarybutyloxycarbonyl; Fmoc, fluorenylmethoxy-carbonyl; Cbz, benzyloxycarbonyl; and Troc, 2,2,2-trichloroethoxycarbonyl.

The present invention has several benefits and advantages.

A salient benefit of the invention is that the combination immunization provides synergistic results in inhibiting diseased cell growth.

An advantage of the invention is that the combination immunization provides T cell help that virus- and bacteria-free vaccines have often lacked.

Another benefit that the invention provides is that those skilled in the art have been finding, studying and publishing formulas of disease-related immunogens that have been ultimately unsuccessful since the early 1980's but can now be successfully put to use.

Still further benefits and advantages of the invention will be apparent to the skilled worker from the disclosures that follow.

### DETAILED DESCRIPTION OF PREFERRED EMBODIMENTS

The present invention contemplates a compound of Formula I, below, wherein R¹, R² and R³

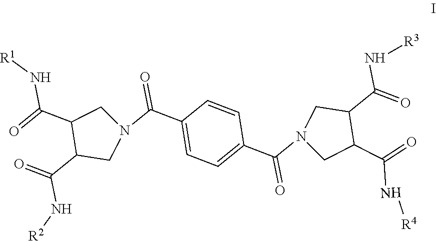

are the same or different and are a trans-2-phenylcyclopropyl, or a trans-2-(4-fluorophenyl)-cyclopropyl group. R⁴ is a composite of (a) a hydrocarbyl group bonded to the depicted amido nitrogen atom and (b) a substituent group bonded thereto as discussed below. The R⁴ hydrocarbyl group has a longest chain of atoms of a total length that is about that of a saturated chain of about 2 carbon atoms (an ethylene group; —CH₂—CH₂—), and a length that is less than that of a saturated chain of about 20 carbon atoms [an eicosylene group].

That hydrocarbyl group includes a methylene group (—CH₂—) that is 1) bonded directly to or 2) bonded indirectly and distal to the amido nitrogen of the depicted —C(O)NH—R⁴ group. That methylene group is also bonded to a substituent group that is i) a phenyl group that includes a substituent selected from the group consisting of a hydroxyl, amino, carboxy [—C(O)OH], C₁-C₆ alkyl carboxylate [—C(O)O-ester], sulfo [—SO₃H], C₁-C₆ alkyl sulfonate [—SO₃-ester], fluoro, azido and an ethynyl |—C≡CH| group, ii) a hydroxyl, a mercaptan, amine, mono- or disubstituted amine, an azido group or iii) to an oxygen, nitrogen or sulfur atom that is part of a substituent group that is selected from the group consisting of an amino acid, ether, ester, carboxylate, carbonate, carbamate, thioether, thioester, thiourea, amido, mono- or disubstituted amide, urea, and a N′-mono or N′,N′-disubstituted urea. The substituent group itself contains a chain of atoms that include zero to four oxygens, zero to two sulfur atoms, and zero to four nitrogen atoms, with the proviso that the sum of the sulfur, nitrogen and oxygen atoms in the substituent chain is at least one, and less than eight. The length of the hydrocarbyl group together with the substituent bonded to the methylene of that hydrocarbyl group is less than that of a saturated chain of about 24 carbon atoms [a tetracosylene group; (—C₂₄H₄₈—)].

In a contemplated compound, each depicted pyrrolidinyldicarboxamido group has the (S,S) configuration. The bonds to the cyclopropyl moiety have a (1S,2R) configuration. Preferably, a contemplated compound has the structural Formula Ia, and the R¹⁻⁴ moieties are as described above

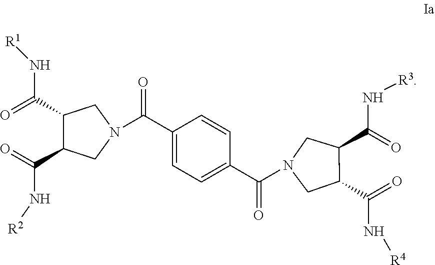

A preferred compound is also a single enantiomer.

An R⁴ group (radical) can be viewed as having been made from two portions as a composite. A first portion is a hydrocarbyl group. A hydrocarbyl group portion of an R⁴ group preferably has a straight alkyl chain or a straight alkyl chain bonded to a terminal phenyl group. The hydrocarbyl group portion of the R⁴ group can have a longest chain of atoms has a total length that is about that of a saturated chain of about 2 carbon atoms (an ethylene group), and a length that is less than that of a saturated chain of about 20 carbon atoms [an eicosylene group]. Preferably, the hydrocarbyl group portion has a saturated chain length of about 6 to about 16 carbon atoms. More preferably still, that saturated chain length is about 10 to about 14 carbon atoms.

It is to be understood that many more atoms can be present in ring structures or substituents that do not constitute part of a carbon chain. Chain length measurement techniques are discussed hereinafter.

The second R⁴ group portion is a substituent group that is covalently bonded to a methylene group (—CH₂—) of the hydrocarbyl portion that is also bonded directly to the amido nitrogen atom of the depicted amide including R⁴ of Formula I or Ia [—C(O)NH—R⁴]. Alternatively, the substituent group can be bonded indirectly and distal to that amido nitrogen. Put differently, the substituent group can alternatively be bonded to a methylene group that is down the hydrocarbyl chain from that amido nitrogen atom. Preferably, that substituent-bonded methylene group is the last carbon atom in the hydrocarbyl chain.

The constituents of the substituent group portion of the R⁴ group can be extremely varied as can be seen in the disclosures that follow. In some embodiments the substituent is a substituted phenyl group.

A phenyl group is a hydrocarbyl group, but because when present a phenyl is itself substituted and because the distinction between the hydrocarbyl and substituent portions of the R⁴ is thought to be clearer, phenyl group is deemed a substituent herein. A phenyl group portion of a substituent group itself can include a substituent selected from the group consisting of a hydroxyl, amino, carboxy, C₁-C₆ alkyl carboxylate (ester), sulfo, C₁-C₆ alkyl sulfonate (ester), fluoro, azido and an ethynyl group.

A second substituent portion of the R⁴ group can be a hydroxyl, a mercaptan, amine, mono- or disubstituted amine, or an azido group.

A third substituent portion bonded to the before-discussed methylene group (—CH₂—) of the hydrocarbyl portion can include an oxygen, nitrogen or sulfur atom that links a remaining part of a substituent group selected from the group consisting of an ether, amino acid, ester, carbonate, carbamate, thioether, thioester, thiourea, amido, mono- or disubstituted amide, urea, and a N′-mono or N′,N′-disubstituted urea. This contemplated substituent group contains a chain of atoms that includes zero to four oxygens, zero to two sulfur atoms, and zero to four nitrogen atoms, with the proviso that the sum of the sulfur, nitrogen and oxygen atoms in the substituent is at least one, and not greater than eight.

The length of the hydrocarbyl group together with the substituent bonded to the methylene of that hydrocarbyl group is greater than a saturated chain of two carbon atoms and less than that of a saturated chain of about 24 carbon atoms [a tetracosylene group].

A contemplated radical chain length is measured along the longest linear atom chain in the radical, following the skeletal atoms around a ring where necessary. Each atom in the chain, e.g. carbon, oxygen, sulfur or nitrogen, is presumed to be carbon for ease in calculation.

Such lengths can be determined by using published bond angles, bond lengths and atomic radii, as needed, to draw and measure a desired, usually staggered, chain, or by building models using commercially available kits whose bond angles, lengths and atomic radii are in accord with accepted, published values. However, such lengths can be time-consuming to determine.

Preferably, therefore, radical (group) lengths are determined somewhat less exactly by assuming that all atoms have bond lengths of saturated carbon, that unsaturated bonds have the same lengths as saturated bonds and that bond angles for unsaturated bonds are the same as those for saturated bonds, although the above-mentioned modes of measurement are preferred. Because of the inherent lack of exactness, lengths are referred to as being “about” a stated length using bond angles, and bond lengths of saturated, single carbon-to-carbon bonds. For example, a phenyl or pyridyl group has a length of a four carbon chain, as does a propoxy group, whereas a biphenyl group has a length of about an eight carbon chain using such a measurement mode.

In addition, an R⁴ group when rotated about an axis drawn through the depicted amido nitrogen-position of Formula I or Ia and the 4-position of a 6-membered ring or the substituent-bonded 3- or 4-position of a 5-membered ring defines a three-dimensional volume whose widest dimension has the width of about one furanyl ring to about two phenyl rings in a direction transverse to that axis to rotation. Thus, a t-butyl group is within that volume whereas the fluorenylmethyl group of an Fmoc amine blocking group extends outside of that volume when so rotated, thereby excluding Fmoc as part of a contemplated substituent group.

### Pharmaceutical Composition and Methods

A contemplated Compound of Formula I or Ia, and of the sub-generic formulas thereof, can also be used in the manufacture of a medicament (pharmaceutical composition). When so used, a contemplated compound of Formula I or Ia, is present dissolved or dispersed in a pharmaceutically acceptable diluent (or carrier) in an amount (or at a concentration) effective to induce release of TNF-α from in vitro cultured human PMA differentiated THP-1 cells.

One use for such a composition is as an adjuvant for a vaccine or inducing an immune response in cultured cells. As such, an improved method of enhancing an immunogen-specific humoral immune response is contemplated that comprises contacting immune cells with an adjuvant-effective amount of a compound of Formula I or Ia, and an immunogen to which that response is to be enhanced. In a live mammal, this method is a method of vaccinating in which a mammal in need of vaccination is administered an effective amount of an immunogen and an effective amount of a compound of Formula I or Ia, as an adjuvant. Here, the improvement comprises using a Compound of Formula I or Ia, or its pharmaceutically acceptable salt as the adjuvant.

For example, studies illustrated elsewhere herein, a contemplated compound acts as a robust in vivo adjuvant or TLR1/TLR2 agonist that evoked a potent TLR2-dependent adjuvant activity in vivo in mice at 200 μg (i.m.) when co-injected with ovalbumin as immunogen by an intramuscular route. In addition, diprovocim-1 did not display the overt toxicity that is characteristic of LPS administration when used as an adjuvant.

A contemplated composition also typically contains pharmaceutically acceptable salts, buffers and the like excipients that collectively are referred to as pharmaceutically (or physiologically) acceptable diluents or carriers as compared to those that can be present in a composition that is not intended for pharmaceutical use, as in an in vitro assay.

A compound of the invention can be provided for use by itself, or as a pharmaceutically acceptable salt. A contemplated Compound of Formula I, an aniline, is a weak base. Parental anilinium ion has a reported pKa value of 4.6. A carboxyl group is also present in the molecule that is preferably esterified, but can be present as a salt.

Exemplary salts useful for a contemplated compound include but are not limited to the following: sulfate, hydrochloride, hydrobromides, acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenyl-propionate, picrate, pivalate, propionate, succinate, tartrate, thiocyanate, tosylate, mesylate and undecanoate. Salts of the carboxylate group include sodium, potassium, magnesium, calcium, aluminum, ammonium, and the many substituted ammonium salts.

The reader is directed to Berge, J. Pharm. Sci. 1977 68(1):1-19 for lists of commonly used pharmaceutically acceptable acids and bases that form pharmaceutically acceptable salts with pharmaceutical compounds.

In some cases, the salts can also be used as an aid in the isolation, purification or resolution of the compounds of this invention. In such uses, the acid used and the salt prepared need not be pharmaceutically acceptable.

As is seen from the data that follow, a contemplated compound is active in in vivo and in in vitro assay studies at micromolar amounts. When used in an assay such as an in vitro assay, a contemplated compound is present in the composition in an amount that is sufficient to provide a concentration of about 10 μM to about 100 μM to contact cells to be assayed.

A contemplated pharmaceutical composition contains an effective amount of a Compound of Formula I or Ia or a pharmaceutically acceptable salt thereof dissolved or dispersed in a physiologically (pharmaceutically) acceptable carrier. In some embodiments, an adjuvant effective (TLR2 agonist effective) amount is utilized. Such a composition can be administered to mammalian cells in vitro as in a cell culture to contact those cells, or the cells can be contacted in vivo as in a living, host mammal in need.

When used as a vaccine adjuvant, a Compound of Formula I or Ia is preferably administered together with the selected immunogen. Both components are preferably present together in a single composition. However, the two ingredients can be present in separately administered compositions, and those separate compositions can be administered up to about one to about two hours apart. It is preferred when two separate compositions are administered, that they be administered as close together in time as possible.

Usually, a Compound of Formula I or Ia contemplated here is administered parenterally in vivo in a weight amount per square meter of the recipient's body surface area (bsa). For adults, this amount is typically about 1 to about 20 mg/m² bsa, and about one-half those amounts for children

A contemplated composition is typically administered in vivo to a subject in need thereof a plurality of times within one month, such as daily or weekly, and can be administered over a period of several months to several years. More usually, a contemplated composition is administered a plurality of times over a course of treatment.

A contemplated pharmaceutical composition can be administered orally (perorally) or parenterally, which is preferred, in a formulation containing conventional nontoxic pharmaceutically acceptable carriers, adjuvants, and vehicles as desired. The term parenteral as used herein includes subcutaneous injections, intravenous, intramuscular (which is most preferred), intrasternal injection, or infusion techniques. Formulation of drugs is discussed in, for example, Hoover, John E., Remington's Pharmaceutical Sciences, Mack Publishing Co., Easton, Pennsylvania; 1975 and Liberman, H. A. and Lachman, L., Eds., Pharmaceutical Dosage Forms, Marcel Decker, New York, N.Y., 1980.

A contemplated pharmaceutical composition is preferably adapted for parenteral administration. Thus, a pharmaceutical composition is preferably in liquid form when administered, and most preferably, the liquid is an aqueous liquid, although other liquids are contemplated as discussed below, and a presently most preferred composition is an injectable preparation.

Thus, injectable preparations, for example, sterile injectable aqueous or oleaginous solutions or suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline.

Other liquid pharmaceutical compositions include, for example, solutions suitable for parenteral administration. Sterile water solutions of a Compound of Formula I or Ia or sterile solution of a Compound of Formula I or Ia in solvents comprising water, ethanol, or propylene glycol are examples of liquid compositions suitable for parenteral administration. In some aspects, a contemplated Compound of Formula I or Ia is provided as a dry powder that is to be dissolved in an appropriate liquid medium such as sodium chloride for injection prior to use.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of an injectable composition. Dimethyl acetamide, surfactants including ionic and non-ionic detergents, polyethylene glycols can be used. Mixtures of solvents and wetting agents such as those discussed above are also useful.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. The amount of a contemplated Compound of Formula I or Ia in a solid dosage form is as discussed previously, an amount sufficient to provide a concentration of about 10 mM to about 100 mM, preferably about 1 nM to about 50 nM, in the serum or blood plasma. A solid dosage form can also be administered a plurality of times during a one week time period.

In such solid dosage forms, a compound of this invention is ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

A mammal in need of treatment (a subject) and to which a pharmaceutical composition containing a Compound of Formula I or Ia is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

Where an in vitro assay is contemplated, a sample to be assayed such as cells and tissue can be used. These in vitro compositions typically contain water, sodium or potassium chloride, and one or more buffer salts such as and acetate and phosphate salts, Hepes or the like, a metal ion chelator such as EDTA that are buffered to a desired pH value such as pH 4.0-8.5, preferably about pH 7.2-7.4, depending on the assay to be performed, as is well known. Preferably, the pharmaceutical composition is in unit dosage form. In such form, the composition is divided into unit doses containing appropriate quantities of the active compound. The unit dosage form can be a packaged preparation, the package containing discrete quantities of the preparation, for example, in vials or ampules.

In another preferred embodiment, a contemplated Compound of Formula I or Ia is administered as an adjuvant along with one or more immunogenic materials as a vaccine. One such composition is illustrated herein in which olvalbumin was used as the immunogen in the vaccination of C57BL/6J mice.

### Results and Discussion

TLR1/TLR2 not only preferentially binds triacyl lipopeptides but also binds diacyl lipopeptides only weakly, whereas TLR2/TLR6 only binds diacyl lipopeptides. X-ray crystallographic structures of Pam3CSK4 (three lipid chains) bound to hTLR1/TLR2 and Pam2CSK4 (two lipid chains) bound to mTRL2/TLR6 have been disclosed.⁹ These studies revealed that the amide lipid chain of Pam3CSK4 inserts into a TLR1 hydrophobic pocket and the remaining two ester lipid chains bind in a TLR2 hydrophobic channel, filling a continuous hydrophobic pocket spanning both proteins at the TLR1/TLR2 heterodimer interface. The diprovocim amide side chains serve the same role as the lipid chains of Pam3CSK4, extending into the hydrophobic pockets spanning TLR1/TLR2.

However, the symmetrical diprovocims (e.g., diprovocim-1) contain four and not three such groups. Models of diprovocim-1 bound to TLR1/TLR2 based on a related X-ray crystal structure and a complementary mutagenesis analysis of the binding site indicate that three of the side chains mimic the three lipid chains of the natural agonists and bind the hydrophobic pockets of TLR1/TLR2 whereas the fourth binds the exterior of the heterodimer interface and extends toward the surrounding solvent.¹⁶ This fourth side chain was chosen to achieve our objectives, providing both a potential functionalized conjugation site and site that might improve murine activity without impacting the already superb human activity.

The initial experimental basis for the studies rested with our prior observation that one of the four side chains in diprovocim-1 could be replaced with a straight chain hydrophobic side chain. That side chain displayed a well-behaved chain length dependence (Table, below), culminating in optimal potency and efficacy with chain lengths of 12-14 that matched the activity of diprovocim-1 (1).¹⁵

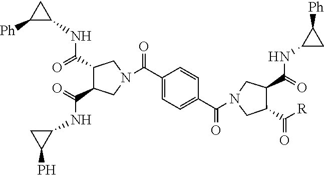

| | EC₅₀ (nM) | EC₅₀ (nM) |
|---|---|---|
| Compound | (THP-1)^{a} | (mouse)^{b} |
| Diprovocim-1 | 0.11 (100) | 1.3 (100) |
| R = OMe | 15 (100)^{c} | 640 (80)_{c} |
| 2, R = OH | 200 (110) | >5000 |
| R = NHCH₂CH₃ | 17 (90) | 360 (130) |
| R = NH(CH₂)₃CH₃ | 2 (120) | 30 (150) |
| R = NH(CH₂)₅CH₃ | 0.25 (100) | 80 (150) |
| R = NH(CH₂)₇CH₃ | 0.33 (100) | 540 (240) |
| R = NH(CH₂)₉CH₃ | 0.1 (100) | 650 (260) |
| R = NH(CH₂)₁₁CH₃ | 0.1 (110) | 2.1 (100) |
| R = NH(CH₂)₁₃CH₃ | 0.1 (150) | 1.1 (100) |
| R = NH(CH₂)₁₅CH₃ | 0.5 (110) | 2.6 (75) |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

The candidate TLR1/TLR2 agonists were assessed for potency (EC₅₀) and efficacy (amount relative to diprovocim-1 as 100%) by measurement of the stimulated release of TNF-α from both differentiated THP-1 cells (human) and thioglycolate-elicited mouse peritoneal macrophages (murine) as detailed in prior studies leading the discovery and characterization of the diprovocims.¹⁵

In initial studies, two series of side chain replacements of varied length that terminate in either a methyl ester or primary alcohol were prepared by coupling the carboxylic acid Compound 2¹⁵ with the corresponding side chain free amine. As seen from Table 1, below, each series recapitulated the observation that potency improved significantly with increasing hydrophobic chain length (12>10>8>6) with each Compounds 3 and 7 (12 carbon length) matching the activity of diprovocim-1.

Although neither the potency nor efficacy improved, it was significant that useful polar functionality could be incorporated at the side chain terminus. Examination of the corresponding carboxylic acid Compound 11 led to a substantial reduction in activity, whereas simple amides (Compounds 12 and 13) and the corresponding amine Compound 16 led only to modest reductions in the activity.

Notably, and analogous to subsequent observations, the Boc protected amine Compound 17 also exhibited good activity approaching that of diprovocim-1 (Compound 1) and the methyl ester and alcohol (Compounds 3 and 7). Combined, these studies indicate that a hydrophobic side chain bearing a terminal ester, alcohol, amine, carbamate, and perhaps amide maintain the activity of the potent diprovocims and offer opportunities for further functionalization or extension.

TABLE 1

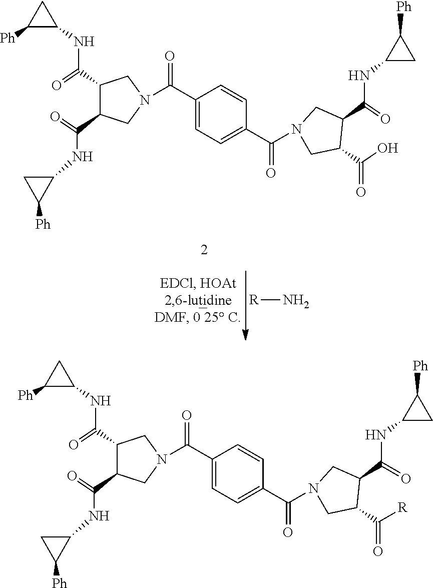

| | | EC₅₀ (nM) | EC₅₀ (nM) |
|---|---|---|---|
| Compound, R = | | (THP-1)^{a} | (mouse)^{b} |
| 1, Diprovocim-1 | | 0.11 (100) | 1.3 (100) |
| 3, NH(CH | | | 1.4 (100)_{c} |
| 4, NH(CH₂)₁₁CO₂Me | | 0.20 (100)^{c} | |
| 5, NH(CH₂)₉CO₂Me | | 0.37 | 17 |
| 6, NH(CH₂)₇CO₂Me | | 3.8 | 240 |
| | ₂)₅CO₂Me | 45 | 1100 |
| 7, NH(CH | | | |
| 8, NH(CH₂)₁₁CH₂OH | | 0.21 (90) | 1.2 (110) |
| 9, NH(CH₂)₉CH₂OH | | 0.5 | 50 |
| | ₂)₇CH₂OH | 4.3 | 93 |

| | | |
|---|---|---|
| 10, NH(CH₂)₅CH₂OH | 90 | 180 |
| 11, NH(CH₂)₁₁CO₂H | 3.4 (100) | 280 (110) |
| 12, NH(CH₂)₁₁CONH₂ | 0.9 (110) | 60 (120) |
| 13, NH(CH₂)₁₁CONHMe | 1 (120) | 60 (150) |
| 14, NH(CH₂)₁₁CONHCH₂CO₂tBu | 20 (100) | 25 (70) |
| 15, NH(CH₂)₁₁CONHCH₂CO₂H | 200 (110) | 1200 (30) |
| 16, NH(CH₂)₁₁CH₂NH₂ | 0.8 (140) | 10 (130) |
| 17, NH(CH₂)₁₁CH₂NHBoc | 0.18 (70) | 2.8 (120) |
| 18, NH(CH₂)₁₁CH₂NHCOCH₂NHBoc | 0.33 (90) | 170 (90) |
| 19, NH(CH₂)₁₁CH₂C≡CH | 0.04 (120) | 1.3 (180) |
| 20, NH(CH₂)₁₁CH₂N₃ | 0.07 (30) | 0.6 (50) |
| 21, NH(CH₂)₁₁CH₂SH | 0.5 | 5 |
| 22, NHCH₂Ph | 7 | 50 |
| 23, NH(CH₂)₂Ph | 1.5 | 30 |
| 24, NHCH₂Ph-4F | 2 | 30 |
| 25, NH(CH₂)₂Ph-4F | 0.5 | 1 |
| 26, NH(CH₂)₈Ph-4OH | 0.15 (90) | 2.6 (380) |
| 27, NH(CH₂)₈Ph-4NH₂ | 0.30 (100) | 6.0 (250) |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

In addition, three functional groups commonly used in conjugation studies (Compounds 19-21; alkyne, azide, and thiol) were found to be well tolerated at the terminus of the 12-carbon hydrophobic chain where the alkyne Compound 19 not only matched or perhaps exceeded the potency of diprovocim-1, but also exhibited a small improvement in the response efficacy. Although not extensively examined, incorporation of a hydrophobic aryl versus straight chain linker was also well tolerated although this was only briefly explored (Compounds 22-27). Within this small series, the activity improved with increasing side chain length with Compounds 26 and 27 approaching the activity of diprovocim-1 and further functionalized with a terminal free phenol or aniline capable of conjugation.

Notably, the efficacy in the murine system employing mouse macrophages exhibited a welcomed substantial increase in efficacy with both Compounds 26 and 27. Finally, introduction of polar atoms in the linker chain with Compounds 28-31, incorporating three repeating ethylene glycol units into the 12-atom linker that were also prepared from Compound 2 by amide coupling with the corresponding amine, led to substantial losses in activity (>1000-fold), highlighting the preferential hydrophobic character required of the side chain region (Table 2).

TABLE 2

| 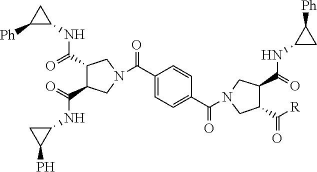 | | |
|---|---|---|
| | EC₅₀ (nM) | EC₅₀ (nM) |
| Compound, R = | (THP-1)^{a} | (mouse)^{b} |
| 28, NHCH₂CH₂(OCH₂CH₂)₃CO₂tBu | 3.1 | 500 |
| 29, NHCH₂CH₂(OCH₂CH₂)₃CO₂Me | 40 (110)^{c} | >500 |
| 30, NHCH₂CH₂(OCH₂CH₂)₃OH | 2000 | >5 μM |
| 31, NHCH₂CH₂(OCH₂CH₂)₃CO₂H | 100 | >5 μM |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

The alcohol Compounds 7 was chosen as the PGP-functionalized side chain to examine in detail for further derivatization (Table vv) by direct acylation. Simple acetylation with Compounds 32 had little further impact on potency although a small improvement was observed in the human THP-1 cell line. Notably, a small improvement was observed in the efficacy (extent of response) in both the human and murine cell lines, suggesting this may provide an avenue to improve the murine efficacy.

Among the earliest such compounds examined, acylation with glycine protected as its Boc derivative to provide Compound 35 afforded an exciting new lead. Its activity and efficacy essentially match that of diprovocim-1 in human THP-1 cells, exhibiting an EC₅₀ of 140 pM with an efficacy of 100% that of diprovocim-1. Even more significantly, it displayed a superb EC₅₀ of 750 pM in mouse macrophages, being 2-fold more active than diprovocim-1, and it exhibited a remarkably improved efficacy of 550% that of diprovocim-1, achieving a key objective of the studies. As a result, and because it became a key compound in the studies, we have come to refer to Compound 35 as diprovocim-X (diprovocim-10), reflecting its chronological discovery.¹⁵

A series of glycine derivatives Compounds 36-42 all exhibited superb potency and good or improved murine efficacy with the exception of the large Fmoc derivative Compound 38. None exceeded the combined properties of diprovocim-X (Compound 35) although the structurally related Troc derivative Compound 37 most closely approached the combined properties, suggesting the size and hydrophobic character of the group is responsible for the improved murine efficacy.

Even the acylated glycine as its free amine (Compound 43), its dimethyl derivative (Compound 44), the corresponding alcohol Compound 45, and its benzyl ether (Compound 46) displayed superb or even improved potency [EC₅₀ 50-200 pM (THP-1) and 0.7-5 nM (mouse macrophages)], while maintaining (THP-1) or slightly improving (mouse macrophages) on the efficacy of diprovocim-1.

Non hydrolytically labile variations on diprovocim-X derived by removing the ester carbonyl affording the ether Compound 48, examination of the similar ether Compound 49, and replacement of the ester with the carbamate Compound 50 provided interesting analogues that deserve comparative examination in instances where questions on the in vivo stability of Compound 35 or its conjugates might arise.

Interestingly, the amide variant Compound 17 of diprovocim-X did not display this increased or improved murine activity, highlighting the importance of the ester 0 versus an amide NH. Further, our studies have shown that even large substituents at this terminal region are well tolerated (not shown) and the 4-substituted benzyl ethers Compounds 51 and 52 are representative of this feature, displaying superb activity and maintained or improved efficacy as seen in Table 3, below.

TABLE 3

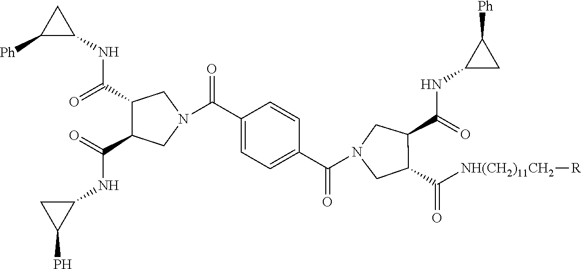

| | EC₅₀ (nM) | EC₅₀ (nM) |
|---|---|---|
| Compound, R = | (THP-1)^{a} | (mouse)^{b} |
| 1, Diprovocim-1 | 0.11 (100) | 1.3 (100) |
| 7, OH | 0.21 (90)^{c} | 1.2 (110)_{c} |
| 32, OAc | 0.14 (130) | 1.5 (140) |
| 33, OCOCH₂CN | 0.25 (100) | 4.5 (120) |
| 34, OCOCH₂N₃ | 0.21 (100) | 3.7 (120) |
| 35, OCOCH₂NHBoc | 0.14 (100) | 0.75 (550) |
| 36, OCOCH₂NHCBz | 0.10 (100) | 40 (410) |
| 37, OCOCH₂NHTroc | 0.27 (100) | 3.4 (300) |
| 38, OCOCH₂NHFmoc | 56 (100) | 375 (170) |
| 39, OCOCH₂NHCO₂Me | 0.38 (100) | 6.0 (120) |
| 40, OCOCH₂NHCOCH₃ | 0.10 (90) | 5.0 (180) |
| 41, OCOCH₂NHCOCF₃ | 0.70 (100) | 3.5 (100) |
| 42, OCOCH₂NHCOtBu | 1.8 (110) | 6.2 (110) |
| 43, OCOCH₂NH₂ | 0.07 (100) | 0.75 (100) |
| 44, OCOCH₂NMe₂ | 0.05 (100) | 4.7 (140) |
| 45, OCOCH₂OH | 0.20 (100) | 0.70 (140) |
| 46, OCOCH₂OBn | 0.06 (130) | 5.3 (220) |
| 47, OCOCH₂OtBu | 0.65 (100) | 5.0 (230) |
| 48, OCH₂CH₂NHBoc | 3.0 (100) | 6.3 (220) |
| 49, OCH₂CO₂tBu | 15 (100) | 1.8 (270) |
| 50, OCONHCH₂CO₂Me | 0.20 (90) | 6.0 (90) |
| 51, OCH₂Ph-4NO₂ | 0.25 (110) | 0.85 (230) |
| 52, OCH₂Ph-4NH₂ | 0.26 (90) | 2.0 (130) |
| 53, OCOCH₂N(Me)CH₂C≡CH | 0.59 (40) | 5.3 (100) |
| 54, OCOCH₂OCH₂C≡CH | 0.29 (30) | 4.9 (110) |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

Consistent with this wider tolerability for substituents extending the side chain beyond the functionalized C12 side chain, introduction of a defined ethylene glycol chain often enlisted with conjugation efforts either through acylation of the free alcohol Compound 7 or amide bond formation on the terminal carboxylate of Compounds 3/11 provided potent TLR1/TLR2 agonists as shown in Table 4, below. Notably, Compound 55 suffered only a two-fold reduction in activity relative to diprovocim-1, approaching the activity of the free alcohol Compound 7 itself, and exhibited a modest increase in the efficacy of the murine response.

TABLE 4

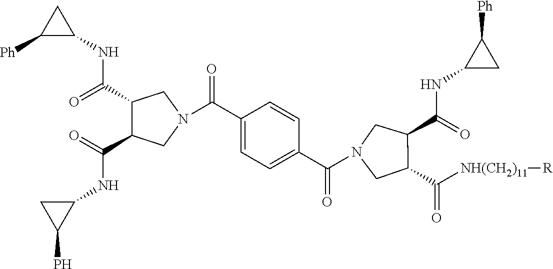

| | EC₅₀ (nM) | EC₅₀ (nM) |
|---|---|---|
| Compound, R = | (THP-1)^{a} | (mouse)^{b} |
| 55, CH₂OCO(CH₂CH₂O)₄CH₂CH₂NHBoc | 0.26 (90) | 3.4 (150) |
| 56, CONH(CH₂CH₂O)₃CH₂CH₂CO₂Bu | 2.8 (100) | 21 (140) |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

Given the impact of the BocNH-glycine extension of the free alcohol Compound 7 that provided diprovocim-X (Compound 35), the acylation of the free alcohol with a full set of BocNH-amino acids was conducted. The results of the study shown in Table 5, below, provided a clear indication that the BocNH-glycine group within Compound 35 plays an integral role in the activity enhancement. Only its replacement with BocNH-L-alanine with Compound 57 approached the activity of Compound 35. All others generally resulted in greater than 10-fold reductions in potency and only sporadic improvements in murine efficacy although all maintained the superb human efficacy. Even the conservative modifications of N-methylation of Compound 35 to provide Compound 70 and replacement of the glycine methylene with a NH to provide the BocNH acyl hydrazide Compound 71 also resulted in this reduction in potency and efficacy.

TABLE 5

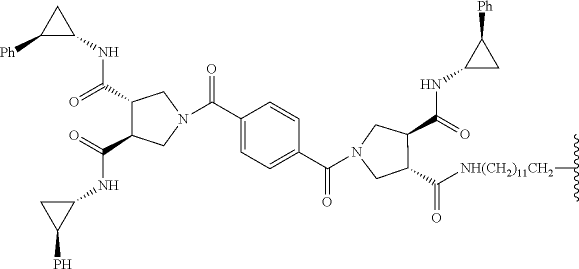

| | | EC₅₀ (nM) | EC₅₀ (nM) |
|---|---|---|---|
| Compound, R = | | (THP-1)^{a} | (mouse)^{b} |
| 1, | Diprovocim-1 | 0.11 (100) | 1.3 (100) |
| 35, | 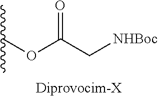 [SMILES: *OC(=O)CNC(=O)OC(C)(C)C] | 0.14 (100)^{c} | 0.75 (550)_{c} |
| 57, | L-Ala | 0.25 (80) | 0.67 (200) |
| 58, | D-Ala | 1.7 (110) | 5.0 (200) |
| | 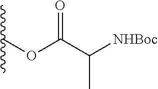 [SMILES: *OC(=O)C(C)NC(=O)OC(C)(C)C] | | |
| 59 | 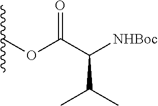 [SMILES: *OC(=O)[C@@H](NC(=O)OC(C)(C)C)C(C)C] | 1.8 (100) | 19 (180) |
| 60 | 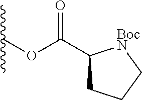 [SMILES: *OC(=O)[C@@H]1CCCN1C(=O)OC(C)(C)C] | 0.94 (90) | 9.2 (100) |
| 61 | 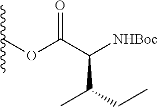 [SMILES: *OC(=O)[C@@H](NC(=O)OC(C)(C)C)[C@@H](C)CC] | 9 (100) | 50 (140) |
| 62 | 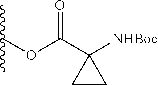 [SMILES: *OC(=O)C1(NC(=O)OC(C)(C)C)CC1] | 5 (100) | 10 (220) |
| 63 | 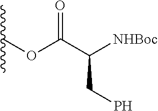 | 40 (100) | 40 (220) |
| 64 | 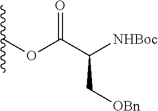 [SMILES: *OC(=O)[C@H](COCc1ccccc1)NC(=O)OC(C)(C)C] | 0.15 (100) | 30 (370) |
| 65 | 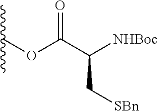 | 18 (100) | 360 (200) |
| 66 | 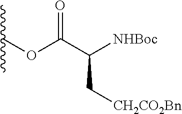 | 39 (100) | 450 (210) |
| 67 | 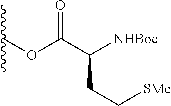 [SMILES: *OC(=O)[C@H](CCSC)NC(=O)OC(C)(C)C] | 2.8 (100) | 44 (150) |
| 68 | 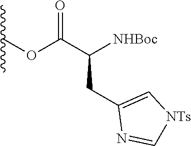 [SMILES: *OC(=O)[C@H](Cc1cn(S(=O)(=O)c2ccc(C)cc2)cn1)NC(=O)OC(C)(C)C] | 17 (90) | 35 (110) |
| 69 | 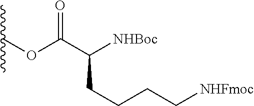 | 280 (100) | 1150 (200) |
| 70 | 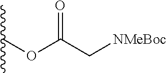 | 1.7 (100) | 17 (110) |
| 71 | 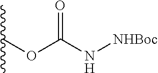 [SMILES: *OC(=O)NNC(=O)OC(C)(C)C] | 5 (100) | 25 (220) |
| 72 | 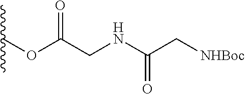 [SMILES: *OC(=O)CNC(=O)CNC(=O)OC(C)(C)C] | 0.52 (110) | 2.5 (110) |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

As these studies were conducted, additional functionalization studies of the alcohol Compound 7, the glycine derivatives 35/44, and its ether variant Compound 48 were pursued in exploration of candidate linker conjugate constructs and/or the consequences of their cleavage. The results of these studies are summarized in Table 6. Acylation of the free alcohol Compound 7 with functionalized p-substituted benzoic acids provided a series of diprovocims, one of which exhibited superb human and murine potency and efficacy (Compound 75) and several of which displayed superb improvements in the murine efficacy (Compounds 73-75).

Although the examples are limited, those terminating in a polar substituent proved substantially more potent than those bearing the paired masked polar substituent, suggesting this region may extend into the surrounding solvent rather than remain in intimate contact with the dimerized receptor. Acylation of the glycine residue of Compound 35 as well as its ether variant Compound 48 with bridging groups that would permit sequential amide bond formations for conjugation with its protein/peptide partners provided analogous observations (Compounds 79-86).

TABLE 6

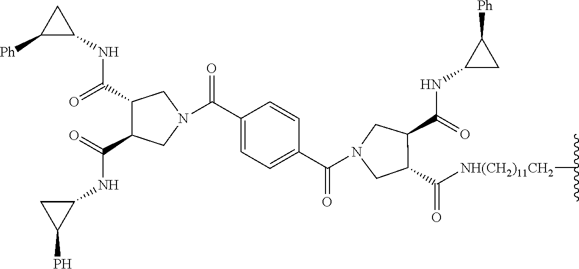

| | | EC₅₀ (nM) | EC₅₀ (nM) |
|---|---|---|---|
| Compound, R = | | (THP-1)^{a} | (mouse)^{b} |
| 1, | Diprovocim-1 | 0.11 (100) | 1.3 (100) |
| 35, | 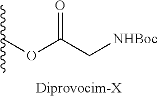 [SMILES: *OC(=O)CNC(=O)OC(C)(C)C] | 0.14 (100)^{c} | 0.75 (550)_{c} |
| 73 | 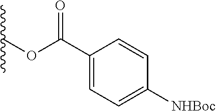 [SMILES: *OC(=O)c1ccc(NC(=O)OC(C)(C)C)cc1] | 0.57 (90) | 30 (540) |
| 74 | 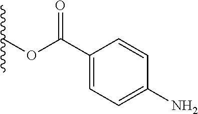 [SMILES: *OC(=O)c1ccc(N)cc1] | 0.05 (100) | 0.67 (300) |
| 75 | 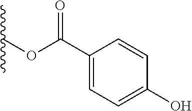 [SMILES: *OC(=O)c1ccc(O)cc1] | 1.7 (100) | 50 (540) |
| 76 | 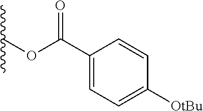 [SMILES: *OC(=O)c1ccc(OC(C)(C)C)cc1] | 27 (<50) | 540 (150) |
| 77 | 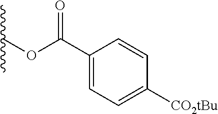 [SMILES: *OC(=O)c1ccc(C(=O)OC(C)(C)C)cc1] | 165 (90) | 785 (190) |
| 78 | 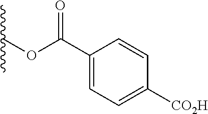 [SMILES: *OC(=O)c1ccc(C(=O)O)cc1] | 0.69 (100) | 2.1 (120) |
| 79 | 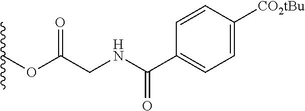 [SMILES: *OC(=O)CNC(=O)c1ccc(C(=O)OC(C)(C)C)cc1] | 6.6 (100) | 465 (130) |
| 80 | 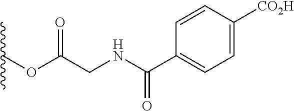 [SMILES: *OC(=O)CNC(=O)c1ccc(C(=O)O)cc1] | 0.17 (100) | 5.0 (60) |
| 81 | 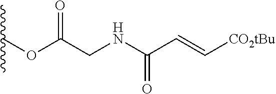 [SMILES: *OC(=O)CNC(=O)/C=C/C(=O)OC(C)(C)C] | 2.0 (90) | 62 (110) |
| 82 | 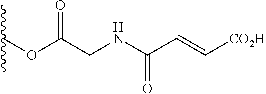 [SMILES: *OC(=O)CNC(=O)/C=C/C(=O)O] | 0.32 (100) | 7.0 (60) |
| 83 | 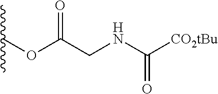 [SMILES: *OC(=O)CNC(=O)C(=O)OC(C)(C)C] | 0.54 (110) | 16 (400) |
| 84 | 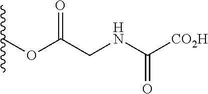 [SMILES: *OC(=O)CNC(=O)C(=O)O] | 0.23 (120) | 6.7 (250) |
| 85 | 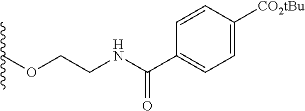 [SMILES: *OCCNC(=O)c1ccc(C(=O)OC(C)(C)C)cc1] | 13.4 (110) | 36 (160) |
| 86 | 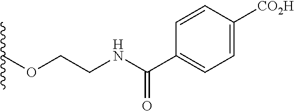 [SMILES: *OCCNC(=O)c1ccc(C(=O)O)cc1] | 2.9 (100) | 49 (50) |

^{a}EC₅₀ TNF-α release from human THP-1 cells.
^{b}EC₅₀ TNF-α release from mouse macrophages.
^{c}Efficacy (magnitude) of response versus diprovocim-1 (100%) shown in parentheses.

Although it does not appear to be a substituent that engages the TLR1 or TLR2 lipid chain hydrophobic pockets, the hydrophobic nature, the length, and terminal functionalization of the fourth side chain can substantially impact the potency and greatly improve the modest murine efficacy (species activity). Most all further side chain extensions beyond that found in diprovocim-X (Compound 35) are well tolerated consistent with expectations that this region extends beyond the intimate TLR1/TLR2 dimerization interface extending into the surrounding solvent.

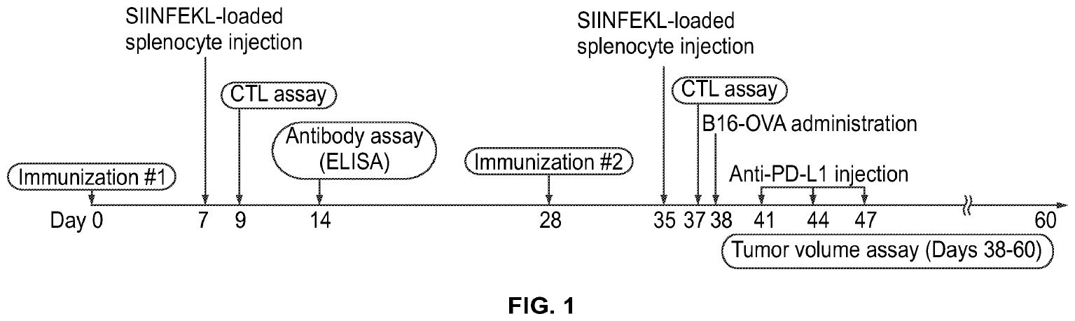

Extensive in vitro characterization of diprovocim-1 (Compound 1) as a TLR1/TLR2 agonist and its downstream signaling was disclosed along with its discovery,¹⁴ indicating it effectively elicits the innate immune response. Because TLR1/TLR2 signaling plays an essential role in the generation of both innate and adaptive immune responses, we investigated whether diprovocim-X (Compound 35), like diprovocim-1 (Compound 1), can also serve as an adjuvant in vivo in mice, indicating stimulation of the adaptive immune response, and whether its improved murine in vitro potency and efficacy translates into a further improved murine in vivo response (FIG. 1).

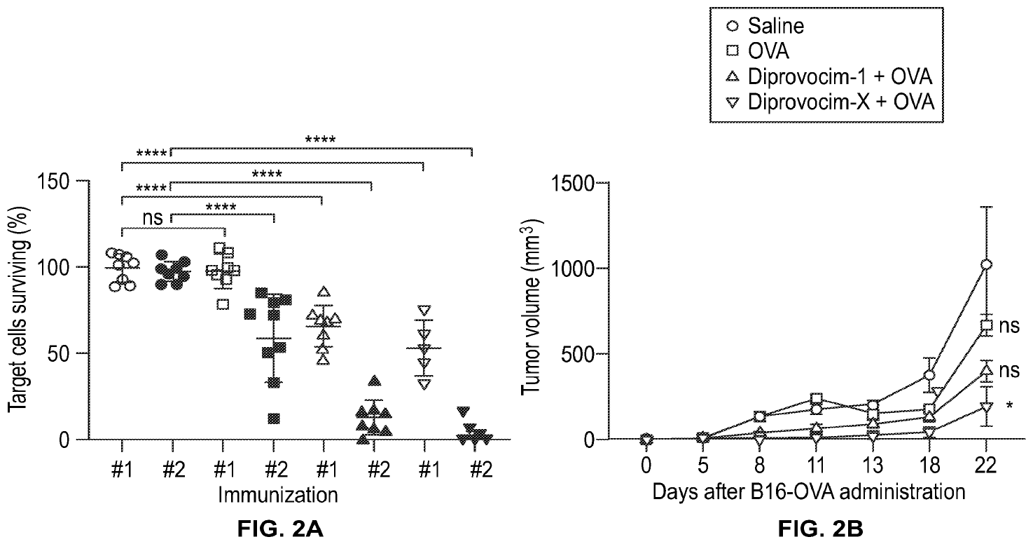

Intramuscular immunization of C57BL/6J mice with ovalbumin (OVA) plus diprovocim-1 or diprovocim-X induced strong in vivo cytotoxicity directed against C57BL/6J splenocytes loaded with the previously-used 8-mer target ovalbumin peptide¹⁴. In vivo cytotoxicity was measured twice: 9 days after the initial immunization and 9 days after a boost, and was notably stronger if two immunizing injections were performed than if only one was performed (FIG. 2A).

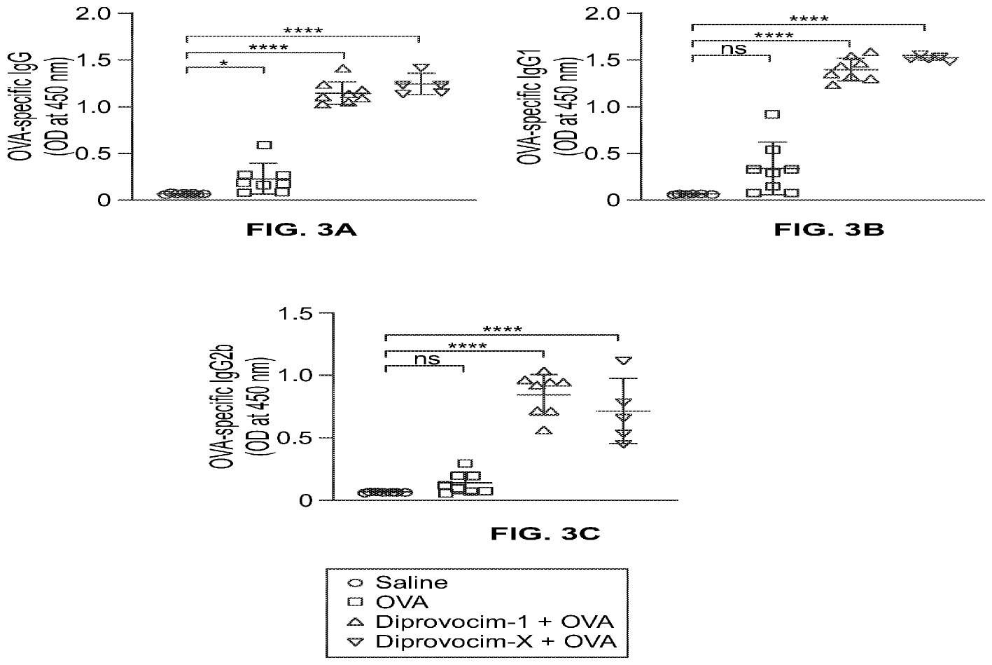

Although no response to OVA alone was measurable after a single injection, a relatively weak response was evident after the boost. Resistance to B16-OVA melanoma, known to be mediated by cytotoxic T lymphocytes, was notably enhanced when inoculation was performed 10 days after the second challenge (FIG. 2B). Fourteen days after a single immunization, OVA-specific circulating antibodies of different subtypes were measured by ELISA (FIGS. 3A, 3B and 3C). Those antibodies showed strong responses to OVA administered in the presence of diprovocim-X and diprovocim-1, but not by itself.

The data demonstrate that diprovocim-1 and diprovocim-X exhibit comparable adjuvant activity in both CD4- and CD-8-dependent responses. Taken together, these data indicate that diprovocim-X (Compound 35) is an adjuvant in mice, matching or surpassing the activity of diprovocim-1, and serve to stimulate the adaptive immune response. Significantly, the study indicates that compounds in this class are of a caliber that exhibit in vivo activity, being active not only on its target proteins and in functional cellular assays, but in vivo in mice as well.

### Conclusions

The diprovocims, which originally emerged from screening a unique chemical library designed to permit cell surface receptor dimerization,¹³ stimulate the innate and adaptive immune responses to co-administered non-immunogenic antigens, act by a well-defined mechanism (TLR1/TLR2 agonist), are easy to produce and structurally manipulate, exhibit exquisite structure-activity relationships, are remarkably potent and efficacious in human and now murine systems, and bear no structural similarity to any known natural or synthetic TLR agonist.¹⁴⁻¹⁶ Herein, we describe studies demonstrating that the fourth side chain of the diprovocims can be replaced with a hydrophobic side chain of a well-defined length, and is terminally functionalized in a manner that results in improvements in potency as well as enhancements in efficacy towards the murine receptor (mTLR1/TLR2), while maintaining remarkable potency and efficacy towards the human receptor (hTLR1/TLR2) as measured by TNF production by macrophages from each species.

We also introduce a site and convenient manner by which covalent linkage to candidate antigens or coupling with targeting or delivery moieties may be conducted without impacting TLR1/TLR2 activation by the diprovocims. These modifications bring powerful capabilities to the diprovocims; in particular, the ability to simultaneously deliver defined adjuvant activity and antigenic peptides in close proximity, thereby minimizing toxicity (e.g., by traditional adjuvant mixtures) and off-target effects (due to stimulation of adaptive immune cells distant from immunostimulated innate immune cells).

The precise immune activation afforded by improvements to the diprovocims described in this work may ultimately permit this adjuvant to be delivered at extremely low (but highly effective, defined) doses, and to be optimized for targeting to antigen presenting cells in lymphoid tissues. The diprovocims, including diprovocim-1 (Compound 1) and now diprovocim-X (Compound 35) detailed herein, represent both user-friendly and highly useful additions to the limited number of TLR agonists. They are attractive starting points for drug discovery, and simple to structurally modify and chemically prepare.¹⁸ As a result, we expect many others will find the diprovocims an attractive class of TLR agonists to work with.

### EXPERIMENTAL

### General Information

Unless otherwise noted, reactions were performed under an atmosphere of Ar using oven-dried glassware. Stainless steel syringes were used to transfer air- and moisture-sensitive liquid reagents. Reactions were monitored by thin-layer chromatography (TLC) on UNIPLATE Silica Gel HLF plates, visualized by UV or fluorescence, or by staining with KMnO₄, anisaldehyde, phosphomolybdic acid or iodine. Column chromatography and preparative thin layer chromatography (PTLC) were used to purify products. Unless otherwise noted, reagents and anhydrous solvents including DMF, CH₂Cl₂, THF and MeOH were purchased from commercial sources, and used as received.

Proton nuclear magnetic resonance (¹H NMR) spectra and carbon nuclear magnetic resonance (¹³C NMR) spectra were recorded on Bruker 400 AVANCE spectrometer (400 and 100 MHz, respectively), Bruker 500 AVANCE spectrometer (500 and 125 MHz, respectively) or Bruker 600 AVANCE spectrometer (600 and 150 MHz, respectively). Chemical shifts (d) for protons are reported in parts per million (ppm) downfield from tetramethylsilane and are referenced to the proton resonance of a residual proton in the NMR solvent (CDCl₃: d=7.26, CD₃OD: d=3.31 or DMSO-d₆: d=2.50). Chemical shifts (d) for carbon are reported in ppm downfield from tetramethylsilane and are referenced to the carbon resonances of the solvent peak (CDCl₃: d=77.23, CD₃OD: d=49.15 or DMSO-d₆: d=39.51). Fluorine nuclear magnetic resonance (¹⁹F NMR) spectra were recorded on a Bruker 400 AVANCE spectrometer (376 MHz). ¹⁹F NMR chemical shifts (d) are reported in ppm upfield from trichlorofluoromethane (0 ppm). NMR data are represented as follows: chemical shift (ppm), multiplicity (s=singlet, d=doublet, t=triplet, q=quartet, p=pent, m=multiplet), coupling constant (J) in Hertz (Hz), integration.

High-resolution mass determinations were obtained either by electrospray ionization (ESI) on a Waters LCT Premier™ mass spectrometer or by matrix-assisted laser desorption/ionization (MALDI). Infrared spectra were measured at a Shimadzu FTIR-84005 Fourier Transform Infrared Spectrometer. Uncorrected melting points were measured on Thomas Hoover Capillary Melting Point apparatus. All tested compounds were >95% pure by HPLC analysis.

### Diprovocim-X (Compound 35):12-((3S,4S)-1-(4-((3S,4S)-3,4-Bis(((1S,2R)-2-phenylcyclopropyl)carbamoyl)-pyrrolidine-1-carbonyl)benzoyl)-4-(((1S,2R)-2-phenyl-cyclopropyl)carbamoyl)-pyrrolidine-3-carboxamido)-dodecyl-2-((tert-butoxycarbonyl)-amino)acetate

A reaction solution of Compound 7 (0.39 g, 0.40 mmol), BocNH-Gly-OH (70 mg, 0.40 mmol), DMAP (49 mg, 0.40 mmol), and EDCI·HCl (0.11 g, 0.60 mmol) in anhydrous DMF (2.0 mL) and CH₂Cl₂ (2.0 mL) was stirred at 40° C. (or rt) for 8 h (or overnight). The reaction was quenched by the addition of aqueous 1 N HCl (1 mL) and the mixture was diluted with H₂O (8 mL). The mixture was extracted with EtOAc (10 mL×3), and the combined organic phase was dried over anhydrous Na₂SO₄, filtered and concentrated under reduced pressure. Flash chromatography (SiO₂, 8-10% MeOH—CH₂Cl₂ gradient) afforded Compound 35 (0.39 g, 86%) as a white solid. [a]_{D}²⁵+44 (c 0.3, HFIP). ¹H NMR (CDCl₃, 400 MHz) δ 7.55-7.51 (m, 4H), 7.38 (br, 1H), 7.29-7.07 (m, 15H), 6.78 (br, 1H), 6.69 (br, 1H), 5.07 (br, 1H), 4.14-4.10 (m, 2H), 3.89-3.88 (m, 2H), 3.81-3.61 (m, 8H), 3.30-3.21 (m, 4H), 3.03-2.82 (m, 5H), 2.06-1.95 (m, 3H), 1.62-1.60 (m, 2H), 1.44 (s, 11H), 1.28-1.07 (m, 22H). ¹³C NMR (CDCl₃, 150 MHz) δ 171.24, 171.21, 171.17, 170.7, 170.6, 170.1, 170.0, 169.9, 169.3, 167.95, 167.92, 167.84, 167.80, 155.3, 139.8, 137.43, 137.38, 137.24, 137.19, 127.98, 127.96, 127.0, 126.9, 125.9, 125.8, 125.7, 79.5, 65.1, 51.5, 51.4, 48.4, 48.3, 48.2, 47.2, 47.1, 47.0, 45.2, 45.1, 45.0, 44.8, 42.0, 39.41, 39.39, 31.8, 31.74, 31.69, 29.08, 29.03, 28.97, 28.90, 28.84, 28.78, 28.7, 28.0, 27.9, 26.4, 25.3, 24.3, 24.21, 24.18, 24.1, 15.49, 15.46, 15.2. IR (film) 3279, 2927, 2855, 1716, 1642, 1546, 1435, 1391, 1260, 1168, 1057, 1031, 863, 736, 696 cm⁻¹. HRMS (ESI-TOF) m/z calcd for C₆₆H₈₄N₇O₁₀ [M+H]⁺ 1134.6274, found 1134.6272. HPLC purity analysis (98.65% pure).

### Methyl 12-((3S,4S)-1-(4-((3S,4S)-3,4-bis(((1S,2R)-2-phenylcyclopropyl)carbamoyl)pyrrolidine-1-carbonyl)-benzoyl)-4-(((1S,2R)-2-phenylcyclopropyl)-carbamoyl)-pyrrolidine-3-carboxamido)-dodecanoate (Compound 3)

A solution of Compound 2¹⁵ (119 mg, 0.150 mmol), methyl 12-aminododecanoate hydrochloride¹⁹ (58.5 mg, 0.220 mmol), HOAt (24.5 mg, 0.180 mmol), and 2,6-lutidine (87 μL, 0.75 mmol) in anhydrous DMF (2.0 mL) was cooled to 0° C. and treated with EDCI·HCl (42.2 mg, 0.220 mmol) in one portion. The reaction solution was stirred at 0° C. for 30 min, warmed to rt and stirred for another 3 h. The reaction was quenched by the addition of aqueous 1 N HCl (1 mL) and diluted with H₂O (2 mL). The mixture was extracted with EtOAc (5 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 3 (112 mg, 74%) as a white solid. ¹H NMR (CDCl₃, 400 MHz) δ 7.52 (s, 4H), 7.37 (br, 1H), 7.28-7.07 (m, 15H), 6.78 (br, 1H), 6.68 (br, 1H), 3.79-3.61 (m, 11H), 3.28-2.98 (m, 6H), 2.89-2.84 (m, 3H), 2.31-2.26 (m, 2H), 2.04-1.98 (m, 3H), 1.62-1.58 (m, 2H), 1.46-1.42 (m, 2H), 1.26-1.11 (m, 20H). HRMS (ESI-TOF) m/z calcd for C₆₀H₇₃N₆O₈ [M+H]⁺ 1005.5484, found 1005.5484.

### Methyl 10-((3S,4S)-1-(4-((3S,4S)-3,4-bis(((1S,2R)-2-phenylcyclopropyl)carbamoyl)pyrrolidine-1-carbonyl)-benzoyl)-4-(((1S,2R)-2-phenylcyclopropyl)-carbamoyl)-pyrrolidine-3-carboxamido)-decanoate (Compound 4)

A solution of 2¹⁵ (20 mg, 0.025 mmol), methyl 10-aminodecanoate hydrochloride (8.9 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 4 (11 mg, 45%) as a white solid. ¹H NMR (CDCl₃, 400 MHz) δ 7.53 (s, 4H), 7.40 (br, 1H), 7.32 (br, 1H), 7.27-7.07 (m, 15H), 6.80 (br, 1H), 6.73 (br, 1H), 3.80-3.63 (m, 11H), 3.31-2.97 (m, 6H), 2.91-2.82 (m, 3H), 2.30-2.25 (m, 2H), 2.05-1.95 (m, 3H), 1.62-1.56 (m, 2H), 1.49-1.41 (m, 2H), 1.27-1.07 (m, 16H). HRMS (ESI-TOF) m/z calcd for C₅₈H₆₉N₆O₈ [M+H]⁺ 977.5171, found 977.5181.

### Methyl 8-((3S,4S)-1-(4-((3S,4S)-3,4-bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-octanoate (Compound 5)

A solution of Compound 2¹⁵ (20 mg, 0.025 mmol), methyl 8-aminooctanoate hydrochloride²⁰ (7.9 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and the solution was diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 5 (17 mg, 72%) as a white solid. ¹H NMR (CDCl₃, 400 MHz) δ 7.53 (s, 4H), 7.45 (br, 1H), 7.40-7.33 (m, 2H), 7.27-7.06 (m, 15H), 6.86-6.81 (m, 1H), 3.79-3.61 (m, 11H), 3.31-2.96 (m, 6H), 2.90-2.80 (m, 3H), 2.28 (q, J=7.6 Hz, 2H), 2.05-1.94 (m, 3H), 1.62-1.56 (m, 2H), 1.48-1.40 (m, 2H), 1.30-1.06 (m, 12H). HRMS (ESI-TOF) m/z calcd for C₅₆H₆₅N₆O₈ [M+H]⁺ 949.4858, found 949.4856.

### Methyl 6-((3S,4S)-1-(4-((3S,4S)-3,4-bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-hexanoate (Compound 6)

A solution of Compound 2¹⁵ (20 mg, 0.025 mmol), methyl 6-aminohexanoate hydrochloride²¹ (6.8 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 6 (17 mg, 74%) as a white solid. ¹H NMR (CDCl₃, 400 MHz) δ 7.53 (s, 4H), 7.44 (br, 1H), 7.34 (br, 2H), 7.27-7.06 (m, 15H), 6.94-6.90 (m, 1H), 3.80-3.59 (m, 11H), 3.30-2.96 (m, 6H), 2.91-2.81 (m, 3H), 2.32-2.26 (m, 2H), 2.05-1.94 (m, 3H), 1.64-1.57 (m, 2H), 1.51-1.43 (m, 2H), 1.37-1.06 (m, 8H). HRMS (ESI-TOF) m/z calcd for C₅₄H₆₁N₆O₈ [M+H]⁺ 921.4545, found 921.4563.

### (3S,4S)-1-(4-((3S,4S)-3,4-Bis(((1S,2R)-2-phenyl-cyclopropyl)carbamoyl)pyrrolidine-1-carbonyl)-benzoyl)-N³-(12-hydroxydodecyl)-N⁴-((1S,2R)-2-phenyl-cyclo-propyl)pyrrolidine-3,4-dicarboxamide (Compound 7)

A solution of Compound 2¹⁵ (127 mg, 0.160 mmol), 12-aminododecan-1-ol hydrochloride (57.1 mg, 0.240 mmol), HOAt (25.9 mg, 0.190 mmol), and 2,6-lutidine (93 μL, 0.80 mmol) in anhydrous DMF (2.0 mL) was cooled to 0° C. and treated with EDCI·HCl (46.0 mg, 0.240 mmol) in one portion. The reaction solution was stirred at 0° C. for 30 min, warmed to rt and stirred for another 3 h. The reaction was quenched by the addition of aqueous 1 N HCl (1 mL) and diluted with H₂O (2 mL). The mixture was extracted with EtOAc (5 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 7 (110 mg, 70%) as a white solid. ¹H NMR (CD₃OD, 400 MHz) δ 7.62 (s, 4H), 7.27-7.06 (m, 15H), 4.03-3.96 (m, 2H), 3.79-3.61 (m, 6H), 3.52 (t, J=6.7 Hz, 2H), 3.34-3.01 (m, 6H), 2.91-2.78 (m, 3H), 2.07-1.92 (m, 3H), 1.53-1.08 (m, 26H). HRMS (ESI-TOF) m/z calcd for C₅₉H₇₃N₆O₇ [M+H]⁺ 977.5535, found 977.5544.

### (3S,4S)-1-(4-((3S,4S)-3,4-Bis(((1S,2R)-2-phenyl-cyclopropyl)carbamoyl)pyrrolidine-1-carbonyl)benzoyl)-N³-(10-hydroxydecyl)-N⁴-((1S,2R)-2-phenylcyclopropyl)-pyrrolidine-3,4-dicarboxamide (Compound 8)

A solution of Compound 2¹⁵ (20 mg, 0.025 mmol), 10-aminodecan-1-ol hydrochloride²² (6.5 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 8 (15 mg, 63%) as a white solid. ¹H NMR (CD₃OD, 400 MHz) δ 7.62 (s, 4H), 7.27-7.06 (m, 15H), 4.03-3.97 (m, 2H), 3.79-3.60 (m, 6H), 3.54-3.50 (m, 2H), 3.34-3.02 (m, 6H), 2.91-2.78 (m, 3H), 2.08-1.91 (m, 3H), 1.53-1.08 (m, 22H). HRMS (ESI-TOF) m/z calcd for C₅₇H₆₉N₆O₇ [M+H]⁺ 949.5222, found 949.5231.

### (3S,4S)-1-(4-((3S,4S)-3,4-Bis(((1S,2R)-2-phenyl-cyclopropyl)carbamoyl)pyrrolidine-1-carbonyl)-benzoyl)-N³—(8-hydroxyoctyl)-N⁴-((1S,2R)-2-phenyl-cyclopropyl)pyrrolidine-3,4-dicarboxamide (Compound 9)

A solution of Compound 2¹⁵ (20 mg, 0.025 mmol), 8-aminooctan-1-ol hydrochloride (5.4 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 9 (17 mg, 74%) as a white solid. ¹H NMR (CD₃OD, 400 MHz) δ 7.62 (s, 4H), 7.27-7.06 (m, 15H), 4.03-3.97 (m, 2H), 3.79-3.61 (m, 7H), 3.52 (q, J=7.1 Hz, 2H), 3.34-3.02 (m, 6H), 2.91-2.77 (m, 3H), 2.08-1.93 (m, 3H), 1.55-1.08 (m, 18H). HRMS (ESI-TOF) m/z calcd for C₅₅H₆₅N₆O₇ [M+H]⁺ 921.4909, found 921.4909.

### (3S,4S)-1-(4-((3S,4S)-3,4-Bis(((1S,2R)-2-phenyl-cyclo-propyl)carbamoyl)pyrrolidine-1-carbonyl)-benzoyl)-N³-(6-hydroxyhexyl)-N⁴-((1S,2R)-2-phenyl-cyclopropyl)-pyrrolidine-3,4-dicarboxamide (Compound 10)

A solution of Compound 2¹⁵ (20 mg, 0.025 mmol), 6-aminohexan-1-ol hydrochloride (4.4 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 10 (18 mg, 81%) as a white solid. ¹H NMR (CD₃OD, 400 MHz) δ 7.62 (s, 4H), 7.27-7.06 (m, 15H), 4.03-3.98 (m, 2H), 3.79-3.61 (m, 7H), 3.56-3.49 (m, 2H), 3.34-3.06 (m, 6H), 2.91-2.79 (m, 3H), 2.09-1.93 (m, 3H), 1.55-1.08 (m, 14H). HRMS (ESI-TOF) m/z calcd for C₅₃H₆₁N₆O₇ [M+H]⁺ 893.4596, found 893.4619.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)carbamoyl)pyrrolidine-3-carboxamido)-dodecanoic acid (Compound 11)

Compound 3 (2.2 mg, 0.00219 mmol) was dissolved in THF/MeOH/H₂O (0.4 mL/0.1 mL/0.1 mL). LiOH (1 mg) was added at 0° C. After the reaction mixture was stirred at room temperature for 3 h, it was poured into aqueous 1 N HCl (2 mL) and EtOAc (3 mL). The aqueous phase was extracted with EtOAc (3 mL), and the combined organic phases were washed with saturated aqueous NaCl (2 mL). The organic phase was dried over Na₂SO₄, filtered and concentrated. PTLC (SiO₂, 8% MeOH/0.05% formic acid/CH₂Cl₂) provided Compound 11 (1.9 mg, 86%). ¹H NMR (600 MHz, DMSO-d₆, mixture of rotamers) δ 8.44-8.27 (m, 3H), 8.06-7.89 (m, 1H), 7.55 (s, 4H), 7.28-7.20 (m, 6H), 7.18-7.09 (m, 6H), 7.08-7.04 (m, 3H), 3.82-3.76 (m, 2H), 3.67-3.61 (m, 2H), 3.54-3.45 (m, 4H), 3.23-2.88 (m, 4H), 2.86-2.72 (m, 3H), 2.20-2.14 (m, 2H), 1.99-1.82 (m, 3H), 1.50-1.43 (m, 2H), 1.42-1.30 (m, 2H), 1.27-1.06 (m, 22H). HRMS (ESI-TOF) m/z calcd for C₅₉H₇₁N₆O₈ [M+H]⁺ 991.5328, found 991.5329.

### (3S,4S)—N³-(12-Amino-12-oxododecyl)-1-(4-((3S,4S)-3,4-bis(((1S,2R)-2-phenylcyclopropyl)-carbamoyl)-pyrrolidine-1-carbonyl)benzoyl)-N⁴-((1S,2R)-2-phenyl-cyclopropyl)pyrrolidine-3,4-dicarboxamide (Compound 12)

Compound 11 (2.7 mg, 0.00272 mmol), ammonium chloride (0.3 mg, 0.00544 mmol), HOAt (0.6 mg, 0.00408 mmol), and 2,6-lutidine (2.9 mg, 0.0272 mmol) were dissolved in anhydrous DMF (0.1 mL). Upon dissolution of the reagents (ca. 5 min) at 0° C., EDCI·HCl (1.6 mg, 0.00816 mmol) was added in one portion at 0° C., and the reaction mixture was stirred at the same temperature for 30 min. The reaction mixture was warmed to rt and stirred for 23 h, after which it was poured into aqueous 1 N HCl (2 mL) and EtOAc (3 mL). The aqueous phase was extracted twice with EtOAc (3 mL), and the combined organic phases were washed sequentially with aqueous 1 N HCl (2 mL), saturated aqueous NaHCO₃ (2 mL), and saturated aqueous NaCl (2 mL). The organic phase was dried over Na₂SO₄, filtered and concentrated. PTLC (SiO₂, 10% MeOH—CH₂Cl₂ gradient) provided Compound 12 (1.8 mg, 67%). ¹H NMR (500 MHz, DMSO-d₆, mixture of rotamers) δ 8.43-8.26 (m, 3H), 8.06-7.88 (m, 1H), 7.56 (s, 4H), 7.28-7.04 (m, 16H), 6.66 (br, 1H) 3.83-3.76 (m, 2H), 3.68-3.61 (m, 2H), 3.55-3.45 (m, 4H), 3.23-2.88 (m, 4H), 2.87-2.72 (m, 3H), 2.03-1.82 (m, 5H), 1.49-1.29 (m, 4H), 1.27-1.06 (m, 22H). HRMS (ESI-TOF) m/z calcd for C₅₉H₇₂N₇O₇ [M+H]⁺ 990.5487, found 990.5484.

### (3S,4S)-1-(4-((3S,4S)-3,4-Bis(((1S,2R)-2-phenylcyclo-propyl)carbamoyl)pyrrolidine-1-carbonyl)-benzoyl)-N³-(12-(methylamino)-12-oxododecyl)-N⁴-((1S,2R)-2-phenyl-cyclopropyl)pyrrolidine-3,4-dicarboxamide (Compound 13)

Compound 11 (2.8 mg, 0.00282 mmol), methylamine hydrochloride (0.4 mg, 0.00564 mmol), HOAt (0.6 mg, 0.00423 mmol), and 2,6-lutidine (3.0 mg, 0.0282 mmol) were dissolved in anhydrous DMF (0.1 mL). Upon dissolution of the reagents (ca. 5 min) at 0° C., EDCI·HCl (1.6 mg, 0.00846 mmol) was added in one portion at 0° C., and the reaction mixture was stirred at the same temperature for 30 min. The reaction mixture was warmed to rt and stirred for 23 h, after which it was poured into aqueous 1 N HCl (2 mL) and EtOAc (3 mL). The aqueous phase was extracted twice with EtOAc (3 mL), and the combined organic phases were washed sequentially with aqueous 1 N HCl (2 mL), saturated aqueous NaHCO₃ (2 mL), and saturated aqueous NaCl (2 mL). The organic phase was dried over Na₂SO₄, filtered and concentrated. PTLC (SiO₂, 10% MeOH—CH₂Cl₂) provided Compound 13 (1.9 mg, 68%). ¹H NMR (500 MHz, CDCl₃, mixture of rotamers) δ 7.54 (s, 4H), 7.31-7.08 (m, 18H), 6.84-6.62 (m, 1H), 5.65-5.56 (m, 1H), 3.86-3.63 (m, 7H), 3.33-3.13 (m, 3H), 3.07-2.96 (m, 2H), 2.94-2.83 (m, 3H), 2.76-2.73 (m, 3H), 2.16-1.96 (m, 5H), 1.63-1.41 (m, 4H), 1.32-1.06 (m, 22H). HRMS (ESI-TOF) m/z calcd for C₆₀H₇₄N₇O₇ [M+H]⁺ 1004.5644, found 1004.5643.

### tert-Butyl (12-((3S,4S)-1-(4-((3S,4S)-3,4-bis(((1S,2R)-2-phenylcyclopropyl)carbamoyl)-pyrrolidine-1-carbonyl)-benzoyl)-4-(((1S,2R)-2-phenylcyclopropyl)carbamoyl)pyrrolidine-3-carboxamido)dodecanoyl)glycinate (Compound 14)

Compound 11 (5.1 mg, 0.00515 mmol), tert-butyl glycinate hydrochloride (1.7 mg, 0.0103 mmol), HOAt (1.1 mg, 0.00773 mmol), and 2,6-lutidine (5.5 mg, 0.0515 mmol) were dissolved in anhydrous DMF (0.1 mL). Upon dissolution of the reagents (ca. 5 min) at 0° C., EDCI·HCl (3.0 mg, 0.0155 mmol) was added in one portion at 0° C., and the reaction mixture was stirred at the same temperature for 30 min. The reaction mixture was warmed to rt and stirred for 23 h, after which it was poured into aqueous 1 N HCl (2 mL) and EtOAc (3 mL). The aqueous phase was extracted twice with EtOAc (3 mL), and the combined organic phases were washed sequentially with aqueous 1 N HCl (2 mL), saturated aqueous NaHCO₃ (2 mL), and saturated aqueous NaCl (2 mL). The organic phase was dried over Na₂SO₄, filtered and concentrated. PTLC (SiO₂, 10% MeOH—CH₂Cl₂) provided Compound 14 (3.6 mg, 63%). ¹H NMR (500 MHz, CDCl₃, mixture of rotamers) δ 7.57 (s, 4H), 7.31-7.05 (m, 18H), 6.81-6.63 (m, 1H), 6.10-6.03 (m, 1H), 3.96-3.89 (m, 2H), 3.85-3.65 (m, 7H), 3.35-3.12 (m, 3H), 3.08-2.97 (m, 2H), 2.96-2.84 (m, 3H), 2.26-2.18 (m, 2H), 2.11-1.98 (m, 3H), 1.68-1.57 (m, 2H), 1.54-1.42 (m, 11H), 1.37-1.08 (m, 22H). HRMS (ESI-TOF) m/z calcd for C₆₅H₈₂N₇O₉ [M+H]⁺ 1104.6168, found 1104.6157.

### (12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)carbamoyl)pyrrolidine-3-carboxamido)-dodecanoyl)glycine (Compound 15)

Compound 14 (2.1 mg, 0.00190 mmol) was dissolved in CH₂Cl₂ (0.2 mL) at rt. TFA (0.2 mL) was added at 0° C., and the mixture was stirred at rt for 2 h. The solvent was removed by a N₂ stream. The residual solids were suspended in MeCN and the solvents removed (repeat twice) to ensure complete removal of the TFA. This process was repeated with CH₂Cl₂. PTLC (SiO₂, 8% MeOH/0.05% formic acid/CH₂Cl₂) provided Compound 15 (2.1 mg, quant). ¹H NMR (500 MHz, DMSO-d₆, mixture of rotamers) δ 8.49-8.31 (m, 3H), 8.10-7.92 (m, 1H), 7.86 (br, 1H), 7.55 (s, 4H), 7.28-7.20 (m, 6H), 7.18-7.03 (m, 9H), 3.82-3.75 (m, 2H), 3.68-3.60 (m, 4H), 3.55-3.45 (m, 4H), 3.24-2.88 (m, 4H), 2.87-2.72 (m, 3H), 2.11-2.06 (m, 2H), 1.99-1.82 (m, 3H), 1.50-1.37 (m, 3H), 1.35-1.29 (m, 1H), 1.27-1.02 (m, 22H). HRMS (ESI-TOF) m/z calcd for C₆₁H₇₄N₇O₉ [M+H]⁺ 1048.5542, found 1048.5524.

### (3S,4S)—N³-(12-Aminododecyl)-1-(4-((3S,4S)-3,4-bis-(((1S,2R)-2-phenylcyclopropyl)carbamoyl)-pyrrolidine-1-carbonyl)benzoyl)-N⁴-((1S,2R)-2-phenylcyclopropyl)-pyrrolidine-3,4-dicarboxamide (Compound 16)

A solution of Compound 17 (21 mg, 0.020 mmol) in 4 N HCl/1,4-dioxane (1.0 mL) was stirred at rt for 1 h. The solvent was removed, and the residue was purified by HPLC using the gradient 0.07% TFA-water/CH₃CN (40%→70%) eluting system to afford the TFA salt of Compound 16 as a white solid. ¹H NMR (CD₃OD, 500 MHz) δ 8.54 (d, J=3.9 Hz, 1H), 8.37 (br, 1H), 7.62 (s, 4H), 7.27-7.06 (m, 15H), 4.02-3.97 (m, 2H), 3.79-3.60 (m, 6H), 3.35-3.03 (m, 6H), 2.91-2.78 (m, 5H), 2.08-1.92 (m, 3H), 1.66-1.59 (m, 2H), 1.53-1.43 (m, 2H), 1.38-1.08 (m, 22H). HRMS (ESI-TOF) m/z calcd for C₅₉H₇₄N₇O₆ [M+H]⁺ 976.5701, found 976.5708.

### tert-Butyl (12-((3S,4S)-1-(4-((3S,4S)-3,4-bis(((1S,2R)-2-phenylcyclopropyl)carbamoyl)-pyrrolidine-1-carbonyl)benzoyl)-4-(((1S,2R)-2-phenylcyclopropyl)carbamoyl)pyrrolidine-3-carboxamido)dodecyl)carbamate (Compound 17)

A solution of Compound 2¹⁵ (20 mg, 0.025 mmol), tert-butyl (12-aminododecyl)carbamate (11 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 17 (22 mg, 82%) as a white solid. ¹H NMR (CDCl₃, 500 MHz) δ 7.54 (d, J=2.8 Hz, 4H), 7.28-7.22 (m, 5H), 7.19-7.08 (m, 10H), 3.80-3.59 (m, 8H), 3.32-2.94 (m, 8H), 2.91-2.82 (m, 3H), 2.06-1.96 (m, 3H), 1.46-1.40 (m, 11H), 1.28-1.07 (m, 24H). HRMS (ESI-TOF) m/z calcd for C₆₄H₈₂N₇O₈ [M+H]⁺ 1076.6225, found 1076.6230.

### tert-Butyl (2-((12-((3S,4S)-1-(4-((3S,4S)-3,4-bis-(((1S,2R)-2-phenylcyclopropyl)carbamoyl)pyrrolidine-1-carbonyl)-benzoyl)-4-(((1S,2R)-2-phenyl-cyclopropyl)-carbamoyl)pyrrolidine-3-carboxamido)-dodecyl)amino)-2-oxoethyl)carbamate (Compound 18)

Step 1: A reaction solution of Compound 17 (21 mg, 0.020 mmol) in MeOH (0.1 mL), CH₂Cl₂ (0.1 mL), and 4 N HCl/1,4-dioxane (1.0 mL) was stirred at rt for 30 min. The solvents were removed under reduced pressure, and the residue was further dried under high vacuum to afford the amine hydrochloride. Step 2: A solution of the amine hydrochloride, N-(tert-butoxycarbonyl)glycine (4.2 mg, 0.024 mmol), HOAt (3.3 mg, 0.024 mmol), and 2,6-lutidine (12 μL, 0.10 mmol) in anhydrous DMF (0.5 mL) was cooled to 0° C. and treated with EDCI·HCl (5.7 mg, 0.030 mmol) in one portion. The reaction solution was stirred at 0° C. for 20 min, warmed to rt and stirred for another 4 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 18 (17 mg, 75%) as a white solid. ¹H NMR (CD₃OD, 400 MHz) δ 8.18 (t, J=5.7 Hz, 1H), 8.01 (t, J=5.7 Hz, 1H), 7.82 (br, 1H), 7.62 (s, 4H), 7.27-7.06 (m, 15H), 6.79 (br, 1H), 4.03-3.97 (m, 2H), 3.79-3.60 (m, 8H), 3.34-3.00 (m, 8H), 2.91-2.78 (m, 3H), 2.09-1.91 (m, 3H), 1.53-1.41 (m, 13H), 1.34-1.07 (m, 22H). HRMS (ESI-TOF) m/z calcd for C₆₆H₈₅N₈O₉ [M+H]⁺ 1133.6440, found 1133.6439.

### (3S,4S)-1-(4-((3S,4S)-3,4-Bis(((1S,2R)-2-phenylcyclo-propyl)carbamoyl)pyrrolidine-1-carbonyl)-benzoyl)-N³-((1S,2R)-2-phenylcyclopropyl)-N⁴-(tetradec-13-yn-1-yl)pyrrolidine-3,4-dicarboxamide (Compound 19)

A solution of Compound 2¹⁵ (20 mg, 0.025 mmol), tetradec-13-yn-1-amine²³ (7.8 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 5-10% MeOH—CH₂Cl₂ gradient) afforded Compound 19 (18 mg, 73%) as a white solid. ¹H NMR (CDCl₃, 500 MHz) δ 7.56-7.52 (m, 4H), 7.28-7.08 (m, 15H), 3.84-3.62 (m, 8H), 3.32-3.10 (m, 4H), 3.06-2.95 (m, 2H), 2.91-2.82 (m, 3H), 2.19-2.15 (m, 2H), 2.06-1.96 (m, 3H), 1.93 (t, J=2.7 Hz, 1H), 1.53-1.06 (m, 26H). HRMS (ESI-TOF) m/z calcd for C₆₁H₇₃N₆O₆ [M+H]⁺ 985.5592, found 985.5602.

### (3S,4S)—N³-(12-Azidododecyl)-1-(4-((3S,4S)-3,4-bis-(((1S,2R)-2-phenylcyclopropyl)carbamoyl)-pyrrolidine-1-carbonyl)benzoyl)-N⁴-((1S,2R)-2-phenylcyclopropyl)-pyrrolidine-3,4-dicarboxamide (Compound 20)

A solution of Compound 2¹⁵ (20 mg, 0.025 mmol), 12-azidododecan-1-amine²³ (8.5 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. PTLC (SiO₂, 10% MeOH—CH₂Cl₂) afforded Compound 20 (16 mg, 64%) as a white solid. ¹H NMR (CD₃OD, 400 MHz) δ 7.61 (s, 4H), 7.27-7.06 (m, 15H), 4.03-3.96 (m, 2H), 3.79-3.60 (m, 6H), 3.35-3.00 (m, 6H), 2.91-2.77 (m, 3H), 2.08-1.91 (m, 3H), 1.58-1.43 (m, 4H), 1.37-1.07 (m, 22H). HRMS (ESI-TOF) m/z calcd for C₅₉H₇₂N₉O₆ [M+H]⁺ 1002.5600, found 1002.5601.

### (3S,4S)-1-(4-((3S,4S)-3,4-Bis(((1S,2R)-2-phenyl-cyclopropyl)carbamoyl)pyrrolidine-1-carbonyl)-benzoyl)-N³-(12-mercaptododecyl)-N⁴-((1S,2R)-2-phenyl-cyclo-propyl)pyrrolidine-3,4-dicarboxamide (Compound 21)

A solution of Compound 2¹⁵ (20 mg, 0.025 mmol), 12-aminododecane-1-thiol hydrochloride²³ (9.5 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. PTLC (SiO₂, 10% MeOH—CH₂Cl₂) afforded 21 (9.0 mg, 36%) as a goose yellow solid. ¹H NMR (CDCl₃, 400 MHz) δ 7.56-7.51 (m, 4H), 7.32 (br, 1H), 7.28-7.08 (m, 15H), 6.81 (d, J=8.1 Hz, 1H), 6.71 (br, 1H), 6.60 (br, 1H), 3.85-3.61 (m, 8H), 3.32-3.09 (m, 4H), 3.06-2.81 (m, 5H), 2.54-2.47 (m, 2H), 2.06-1.96 (m, 3H), 1.62-1.05 (m, 26H). HRMS (ESI-TOF) m/z calcd for C₅₉H₇₃N₆O₆S [M+H]⁺ 993.5312, found 993.5317.

### (3S,4S)—N³-Benzyl-1-(4-((3S,4S)-3,4-bis(((1S,2R)-2-phenylcyclopropyl)carbamoyl)pyrrolidine-1-carbonyl)-benzoyl)-N⁴-((1S,2R)-2-phenylcyclopropyl)pyrrolidine-3,4-dicarboxamide (Compound 22)

A solution of Compound 2¹⁵ (20 mg, 0.025 mmol), phenylmethanamine (8.2 μL, 0.075 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 22 (18 mg, 81%) as a white solid. ¹H NMR (CD₃OD, 400 MHz) δ 8.67 (br, 1H), 8.54 (br, 1H), 8.48 (br, 1H), 8.37 (br, 1H), 7.61 (s, 4H), 7.35-7.05 (m, 20H), 4.52-4.20 (m, 2H), 4.05-3.97 (m, 2H), 3.80-3.61 (m, 6H), 3.42-3.18 (m, 4H), 2.92-2.74 (m, 3H), 2.09-1.87 (m, 3H), 1.26-1.01 (m, 6H). HRMS (ESI-TOF) m/z calcd for C₅₄H₅₅N₆O₆ [M+H]⁺ 883.4183, found 883.4184.

### (3S,4S)-1-(4-((3S,4S)-3,4-Bis(((1S,2R)-2-phenyl-cyclopropyl)carbamoyl)pyrrolidine-1-carbonyl)-benzoyl)-N³-phenethyl-N⁴-((1S,2R)-2-phenylcyclo-propyl)pyrrolidine-3,4-dicarboxamide (Compound 23)

A solution of Compound 2¹⁵ (20 mg, 0.025 mmol), 2-phenylethanamine (9.4 μL, 0.075 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 23 (18 mg, 80%) as a white solid. ¹H NMR (CD₃OD, 400 MHz) δ 8.54 (d, J=3.8 Hz, 1H), 8.37 (t, J=4.1 Hz, 1H), 8.24 (t, J=5.7 Hz, 1H), 8.04 (t, J=5.8 Hz, 1H), 7.64-7.58 (m, 4H), 7.30-7.05 (m, 20H), 4.04-3.92 (m, 2H), 3.80-3.57 (m, 6H), 3.51-3.38 (m, 2H), 3.36-3.15 (m, 4H), 2.92-2.65 (m, 5H), 2.08-1.89 (m, 3H), 1.25-1.05 (m, 6H). HRMS (ESI-TOF) m/z calcd for C₅₅H₅₇N₆O₆ [M+H]⁺ 897.4340, found 897.4338.

### (3S,4S)-1-(4-((3S,4S)-3,4-Bis(((1S,2R)-2-phenylcyclo-propyl)carbamoyl)pyrrolidine-1-carbonyl)-benzoyl)-N³-(4-fluorobenzyl)-N⁴-((1S,2R)-2-phenylcyclopropyl)-pyrrolidine-3,4-dicarboxamide (Compound 24)

A solution of Compound 2¹⁵ (20 mg, 0.025 mmol), (4-fluorophenyl)methanamine (8.6 μL, 0.075 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 24 (18 mg, 80%) as a white solid. ¹H NMR (CD₃OD, 400 MHz) δ 7.60 (s, 4H), 7.32-6.98 (m, 19H), 4.47-4.17 (m, 2H), 4.03-3.98 (m, 2H), 3.79-3.61 (m, 6H), 3.41-3.19 (m, 4H), 2.90-2.72 (m, 3H), 2.08-1.86 (m, 3H), 1.25-0.99 (m, 6H). HRMS (ESI-TOF) m/z calcd for C₅₄H₅₄FN₆O₆[M+H]⁺ 901.4089, found 901.4105.

### (3S,4S)-1-(4-((3S,4S)-3,4-Bis(((1S,2R)-2-phenylcyclo-propyl)carbamoyl)pyrrolidine-1-carbonyl)-benzoyl)-N³-(4-fluorophenethyl)-N⁴-((1S,2R)-2-phenylcyclopropyl)-pyrrolidine-3,4-dicarboxamide (Compound 25)

A solution of Compound 2¹⁵ (20 mg, 0.025 mmol), 2-(4-fluorophenyl)ethanamine (9.8 μL, 0.075 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 25 (18 mg, 79%) as a white solid. ¹H NMR (CD₃OD, 400 MHz) δ 7.64-7.58 (m, 4H), 7.27-6.89 (m, 19H), 4.04-3.93 (m, 2H), 3.80-3.48 (m, 6H), 3.43 (t, J=7.2 Hz, 1H), 3.38-3.15 (m, 5H), 2.90-2.64 (m, 5H), 2.08-1.89 (m, 3H), 1.25-1.05 (m, 6H). HRMS (ESI-TOF) m/z calcd for C₅₅H₅₆FN₆O₆[M+H]⁺ 915.4245, found 915.4246.

### (3S,4S)-1-(4-((3S,4S)-3,4-Bis(((1S,2R)-2-phenylcyclo-propyl)carbamoyl)pyrrolidine-1-carbonyl)-benzoyl)-N³-(8-(4-hydroxyphenyl)octyl)-N⁴-((1S,2R)-2-phenylcyclo-propyl)pyrrolidine-3,4-dicarboxamide (Compound 26)

A solution of Compound 2¹⁵ (20 mg, 0.025 mmol), 4-(8-aminooctyl)phenol²³ (8.3 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 26 (20 mg, 80%) as a white solid. ¹H NMR (CD₃OD, 400 MHz) δ 7.60 (s, 4H), 7.26-7.03 (m, 15H), 6.94 (dd, J=8.3, 4.5 Hz, 2H), 6.67 (d, J=8.5 Hz, 2H), 4.03-3.96 (m, 2H), 3.77-3.59 (m, 6H), 3.34-3.00 (m, 6H), 2.90-2.76 (m, 3H), 2.46 (q, J=8.1 Hz, 2H), 2.08-1.90 (m, 3H), 1.55-1.39 (m, 4H), 1.31-1.06 (m, 14H). HRMS (ESI-TOF) m/z calcd for C₆₁H₆₉N₆O₇ [M+H]⁺ 997.5228, found 997.5220.

### (3S,4S)—N-(8-(4-Aminophenyl)octyl)-1-(4-((3S,4S)-3,4-bis(((1S,2R)-2-phenylcyclopropyl)carbamoyl)-pyrrolidine-1-carbonyl)benzoyl)-N⁴-((1S,2R)-2-phenyl-cyclopropyl)-pyrrolidine-3,4-dicarboxamide (Compound 27)

A solution of Compound 2¹⁵ (20 mg, 0.025 mmol), 4-(8-aminooctyl)aniline²³ (8.3 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 30 min, warmed to rt and stirred for another 3 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 27 (9.0 mg, 36%) as a white solid. ¹H NMR (CD₃OD, 400 MHz) δ 7.61 (d, J=1.9 Hz, 4H), 7.27-7.05 (m, 15H), 6.93 (dd, J=8.0, 5.6 Hz, 2H), 6.71 (dd, J=8.3, 2.6 Hz, 2H), 4.03-3.96 (m, 2H), 3.78-3.59 (m, 6H), 3.34-3.02 (m, 6H), 2.91-2.77 (m, 3H), 2.46 (q, J=8.4 Hz, 2H), 2.09-1.90 (m, 3H), 1.59-1.40 (m, 4H), 1.32-1.06 (m, 14H). HRMS (ESI-TOF) m/z calcd for C₆₁H₇₀N₇O₆[M+H]⁺ 996.5388, found 996.5397.

### tert-Butyl 1-((3S,4S)-1-(4-((3S,4S)-3,4-bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidin-3-yl)-1-oxo-5,8,11-trioxa-2-azatetradecan-14-oate (Compound 28)

Compound 2¹⁵ (6.0 mg, 0.0076 mmol), tert-butyl 3-(2-(2-(2-aminoethoxy)ethoxy)ethoxy)propanoate (3.2 mg, 0.0114 mmol), HOAt (1.2 mg, 0.00912 mmol), and 2,6-lutidine (4.1 mg, 0.0380 mmol) were dissolved in anhydrous DMF (0.1 mL). Upon dissolution of the reagents (ca. 5 min) at 0° C., EDCI·HCl (2.2 mg, 0.0114 mmol) was added in one portion at 0° C., and the reaction mixture was stirred at the same temperature for 30 min. The reaction mixture was warmed to rt and stirred for 3 h, after which it was poured into aqueous 1 N HCl (2 mL) and EtOAc (3 mL). The aqueous phase was extracted with EtOAc (3 mL), and the combined organic phases were washed sequentially with aqueous 1 N HCl (2 mL), saturated aqueous NaHCO₃ (2 mL), and saturated aqueous NaCl (2 mL). The organic phase was dried over Na₂SO₄, filtered and concentrated. PTLC (SiO₂, 10% MeOH—CH₂Cl₂) provided Compound 28 (6.3 mg, 79%). ¹H NMR (500 MHz, CDCl₃, mixture of rotamers) δ 7.57-7.51 (m, 4H), 7.35-6.99 (m, 19H), 3.97-3.41 (m 22H), 3.34-2.96 (m, 4H), 2.93-2.79 (m, 3H), 2.52-2.43 (m, 2H), 2.09-1.95 (m, 3H), 1.46-1.41 (m, 9H), 1.28-1.06 (m, 6H). HRMS (ESI-TOF) m/z calcd for C₆₀H₇₃N₆O₁₁ [M+H]⁺ 1053.5322, found 1053.5334.

### Methyl 1-((3S,4S)-1-(4-((3S,4S)-3,4-bis(((1S,2R)-2-phenylcyclopropyl)carbamoyl)pyrrolidine-1-carbonyl) benzoyl)-4-(((1S,2R)-2-phenylcyclopropyl)carbamoyl)-pyrrolidin-3-yl)-1-oxo-5,8,11-trioxa-2-azatetradecan-14-oate (Compound 29)

A solution of Compound 2¹⁵ (20 mg, 0.025 mmol), methyl 3-(2-(2-(2-aminoethoxy)ethoxy)ethoxy)-propanoate hydrochloride²⁴ (10 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 20 min, warmed to rt and stirred for another 4 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 29 (16 mg, 63%) as a white solid. ¹H NMR (CD₃OD, 400 MHz) δ 7.62 (s, 4H), 7.27-7.06 (m, 15H), 4.03-3.97 (m, 2H), 3.80-3.45 (m, 23H), 3.43-3.17 (m, 4H), 2.91-2.78 (m, 3H), 2.58-2.52 (m, 2H), 2.09-1.92 (m, 3H), 1.28-1.08 (m, 6H). HRMS (ESI-TOF) m/z calcd for C₅₇H₆₇N₆O₁₁ [M+H]⁺ 1011.4868, found 1011.4876.

### (3S,4S)-1-(4-((3S,4S)-3,4-Bis(((1S,2R)-2-phenyl-cyclopropyl)carbamoyl)pyrrolidine-1-carbonyl)-benzoyl)-N³-(2-(2-(2-(2-hydroxyethoxy)ethoxy)ethoxy)-ethyl)-N⁴-((1S,2R)-2-phenylcyclopropyl)-pyrrolidine-3,4-dicarboxamide (Compound 30)

A solution of Compound 2¹⁵ (20 mg, 0.025 mmol), 2-(2-(2-(2-aminoethoxy)ethoxy)ethoxy)ethanol (7.2 mg, 0.0375 mmol), HOAt (4.1 mg, 0.030 mmol), and 2,6-lutidine (15 μL, 0.125 mmol) in anhydrous DMF (0.5 mL) was cooled to 0° C. and treated with EDCI·HCl (7.2 mg, 0.0375 mmol) in one portion. The reaction solution was stirred at 0° C. for 20 min, warmed to rt and stirred for another 4 h. The reaction was quenched by the addition of aqueous 1 N HCl (0.5 mL) and diluted with H₂O (2 mL). The mixture was extracted with EtOAc (4 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 30 (19 mg, 78%) as a white solid. ¹H NMR (CD₃OD, 400 MHz) δ 7.62 (s, 4H), 7.27-7.06 (m, 15H), 4.03-3.98 (m, 2H), 3.79-3.47 (m, 22H), 3.44-3.17 (m, 4H), 2.91-2.78 (m, 3H), 2.08-1.92 (m, 3H), 1.33-1.10 (m, 6H). HRMS (ESI-TOF) m/z calcd for C₅₅H₆₅N₆O₁₀ [M+H]⁺ 969.4762, found 969.4762.

### 1-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidin-3-yl)-1-oxo-5,8,11-trioxa-2-azatetradecan-14-oic acid (Compound 31)

Compound 28 (2.2 mg, 0.00209 mmol) was dissolved in CH₂Cl₂ (0.4 mL) at rt. TFA (0.38 mL), triisopropylsilane (0.02 mL) and H₂O (0.02 mL) was added at 0° C., and the mixture was stirred at rt for 2 h. The solvent was removed by a N₂ stream. The residual solid was suspended in MeCN and the solvent evaporated (repeated twice) to ensure complete removal of the TFA. This process was repeated with CH₂Cl₂. PTLC (SiO₂, 8% MeOH/0.05% formic acid/CH₂Cl₂) provided Compound 31 (2.1 mg, quant). ¹H NMR (500 MHz, DMSO-d₆, mixture of rotamers) δ 8.44-8.27 (m, 3H), 8.18-8.00 (m, 1H), 7.55 (s, 4H), 7.28-7.20 (m, 6H), 7.18-7.10 (m, 6H), 7.08-7.04 (m, 3H), 3.82-3.75 (m, 2H), 3.68-3.62 (m, 2H), 3.61-3.55 (m, 3H), 3.54-3.33 (m, 13H), 3.27-3.07 (m, 6H), 2.87-2.73 (m, 3H), 2.45-2.39 (m, 2H), 1.99-1.82 (m, 3H), 1.20-1.07 (m, 6H). HRMS (ESI-TOF) m/z calcd for C₅₆H₆₅N₆O₁₁ [M+H]⁺ 997.4706, found 997.4708.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)carbamoyl)pyrrolidine-3-carboxamido)dodecyl acetate (Compound 32)

A solution of Compound 7 (9.8 mg, 0.010 mmol) and DMAP (3.7 mg, 0.030 mmol) in anhydrous pyridine (50 μL) was treated with acetic anhydride (50 μL) dropwise at rt. The reaction mixture was stirred at 50° C. overnight (about 18 h), and cooled to rt. The reaction mixture was quenched by the addition of aqueous 10% citric acid (0.5 mL) and diluted with H₂O (0.5 mL). The mixture was extracted with EtOAc (1 mL×3), and the combined organic phase was dried over anhydrous Na₂SO₄, filtered and concentrated under reduced pressure. PTLC (SiO₂, 10% MeOH—CH₂Cl₂) afforded Compound 32 (8.0 mg, 78%) as a white solid. ¹H NMR (CDCl₃, 400 MHz) δ 7.54 (s, 4H), 7.34 (br, 1H), 7.28-7.08 (m, 15H), 6.80 (br, 1H), 6.73 (br, 1H), 6.63 (br, 1H), 4.06-4.02 (m, 2H), 3.85-3.61 (m, 8H), 3.33-3.08 (m, 4H), 3.05-2.81 (m, 5H), 2.06-1.95 (m, 6H), 1.63-1.40 (m, 4H), 1.34-1.05 (m, 22H). HRMS (ESI-TOF) m/z calcd for C₆₁H₇₅N₆O₈ [M+H]⁺ 1019.5646, found 1019.5661.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)dodecyl 2-cyanoacetate (Compound 33)

A solution of 2-cyanoacetic acid (2.6 mg, 0.030 mmol) in anhydrous THF (0.14 mL) was treated with Et₃N (8.4 μL, 0.060 mmol) and 2,4,6-trichloro-benzoyl chloride (7.8 μL, 0.050 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight). The reaction mixture was quenched by the addition of aqueous 1 N HCl (0.2 mL) and diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 33 (7.1 mg, 68%) as a white solid. ¹H NMR (CD₃OD, 400 MHz) δ 7.62 (s, 4H), 7.27-7.07 (m, 15H), 4.18-4.14 (m, 2H), 4.03-3.96 (m, 2H), 3.79-3.60 (m, 6H), 3.35-3.01 (m, 8H), 2.91-2.78 (m, 3H), 2.09-1.91 (m, 3H), 1.69-1.61 (m, 2H), 1.55-1.42 (m, 2H), 1.38-1.07 (m, 22H). HRMS (ESI-TOF) m/z calcd for C₆₂H₇₄N₇O₈ [M+H]⁺ 1044.5599, found 1044.5603.

### 12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)dodecyl 2-azidoacetate (Compound 34)

A solution of 2-azidoacetic acid²⁵ (1.4 mg, 0.014 mmol) in anhydrous THF (0.14 mL) was treated with Et₃N (3.8 μL, 0.027 mmol) and 2,4,6-trichloro-benzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight). The reaction mixture was quenched by the addition of aqueous 1 N HCl (0.2 mL) and diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 34 (8.8 mg, 83%) as a white solid. ¹H NMR (CD₃OD, 400 MHz) δ 7.62 (s, 4H), 7.27-7.06 (m, 15H), 4.17 (t, J=6.6 Hz, 2H), 4.03-3.96 (m, 2H), 3.93 (s, 2H), 3.79-3.60 (m, 6H), 3.35-3.01 (m, 6H), 2.91-2.77 (m, 3H), 2.08-1.91 (m, 3H), 1.68-1.61 (m, 2H), 1.55-1.42 (m, 2H), 1.37-1.07 (m, 22H). HRMS (ESI-TOF) m/z calcd for C₆₁H₇₄N₉O₈ [M+H]⁺ 1060.5660, found 1060.5658.

### 12-((3S,4S)-1-(4-((3S,4S)-3,4-Bis(((1S,2R)-2-phenyl-cyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-(((benzyloxy)carbonyl)-amino)acetate (Compound 36)

A reaction solution of 7 (9.8 mg, 0.010 mmol), CbzNH-Gly-OH (2.1 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 36 (7.9 mg, 68%) as a white solid. ¹H NMR (CDCl₃, 400 MHz) δ 7.56-7.51 (m, 4H), 7.35-7.30 (m, 5H), 7.28-7.08 (m, 15H), 6.75 (br, 1H), 6.64 (br, 1H), 5.43-5.35 (m, 1H), 5.12 (s, 2H), 4.14-4.10 (m, 2H), 3.97-3.95 (m, 2H), 3.81-3.61 (m, 8H), 3.29-3.12 (m, 4H), 3.05-2.82 (m, 5H), 2.06-1.95 (m, 3H), 1.66-1.57 (m, 2H), 1.50-1.40 (m, 2H), 1.28-1.06 (m, 22H). HRMS (ESI-TOF) m/z calcd for C₆₉H₈₂N₇O₁₀ [M+H]⁺ 1168.6123, found 1168.6108.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)carbamoyl)pyrrolidine-3-carboxamido)-dodecyl 2-(((2,2,2-trichloroethoxy)-carbonyl)amino)acetate (Compound 37)

A reaction solution of Compound 7 (9.8 mg, 0.010 mmol), TrocNH-Gly-OH²⁶ (2.5 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight). The reaction was quenched by the addition of 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 37 (7.8 mg, 64%) as a white solid. ¹H NMR (CDCl₃, 400 MHz) δ 7.56-7.52 (m, 4H), 7.32 (br, 1H), 7.28-7.08 (m, 15H), 6.74 (br, 1H), 6.64 (br, 1H), 5.69-5.61 (m, 1H), 4.74 (s, 2H), 4.17-4.12 (m, 2H), 4.00-3.98 (m, 2H), 3.82-3.61 (m, 8H), 3.32-3.12 (m, 4H), 3.02-2.82 (m, 5H), 2.07-1.95 (m, 3H), 1.66-1.60 (m, 2H), 1.51-1.40 (m, 2H), 1.28-1.07 (m, 22H). HRMS (ESI-TOF) m/z calcd for C₆₄H₇₇Cl₃N₇O₁₀ [M+H]⁺ 1208.4797, found 1208.4785.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)carbamoyl)pyrrolidine-3-carboxamido)-dodecyl 2-((((9H-fluoren-9-yl)methoxy)carbonyl)amino)acetate (Compound 38)

A solution of FmocNH-Gly-OH (4.2 mg, 0.014 mmol) in anhydrous THF (0.14 mL) was treated with Et₃N (1.4 μL, 0.010 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred at 40° C. (or rt) for 8 h (or overnight). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 38 (9.8 mg, 78%) as a white solid. ¹H NMR (CDCl₃, 400 MHz) δ 7.76 (d, J=7.5 Hz, 2H), 7.59 (d, J=7.6 Hz, 2H), 7.55-7.51 (m, 4H), 7.39 (t, J=7.5 Hz, 2H), 7.34-7.07 (m, 17H), 5.46-5.39 (m, 1H), 4.39 (d, J=7.1 Hz, 2H), 4.23 (t, J=7.2 Hz, 1H), 4.16-4.12 (m, 2H), 3.98-3.96 (m, 2H), 3.87-3.61 (m, 8H), 3.29-3.13 (m, 4H), 3.05-2.82 (m, 5H), 2.06-1.96 (m, 3H), 1.66-1.58 (m, 2H), 1.51-1.39 (m, 2H), 1.35-1.07 (m, 22H). HRMS (ESI-TOF) m/z calcd for C₇₆H₈₆N₇O₁₀ [M+H]⁺ 1256.6436, found 1256.6462.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)-dodecyl 2-((methoxycarbonyl)amino)acetate (Compound 39)

A reaction solution of Compound 7 (9.8 mg, 0.010 mmol), N-(methoxycarbonyl)glycine (1.3 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 39 (7.8 mg, 71%) as a white solid. ¹H NMR (CDCl₃, 400 MHz) δ 7.56-7.52 (m, 4H), 7.33 (br, 1H), 7.28-7.08 (m, 15H), 6.76 (br, 1H), 6.66 (br, 1H), 5.36 (br, 1H), 4.15-4.11 (m, 2H), 3.95-3.93 (m, 2H), 3.82-3.62 (m, 11H), 3.32-3.12 (m, 4H), 3.06-2.82 (m, 5H), 2.05-1.96 (m, 3H), 1.66-1.61 (m, 2H), 1.48-1.43 (m, 2H), 1.28-1.06 (m, 22H). HRMS (ESI-TOF) m/z calcd for C₆₃H₇₈N₇O₁₀ [M+H]⁺ 1092.5810, found 1092.5787.

### 12-((3S,4S)-1-(4-((3S,4S)-3,4-Bis(((1S,2R)-2-phenyl-cyclopropyl)carbamoyl)pyrrolidine-1-carbonyl)-benzoyl)-4-(((1S,2R)-2-phenylcyclopropyl)-carbamoyl)pyrrolidine-3-carboxamido)-dodecyl 2-acetamidoacetate (Compound 40)

A solution of Compound 2¹⁵ (48 mg, 0.060 mmol), 12-bromododecan-1-amine hydrobromide²⁷ (31 mg, 0.090 mmol), HOAt (9.8 mg, 0.072 mmol), and 2,6-lutidine (35 μL, 0.30 mmol) in anhydrous DMF (0.6 mL) was cooled to 0° C. and treated with EDCI·HCl (17 mg, 0.090 mmol) in one portion. The reaction solution was stirred at 0° C. for 30 min, warmed to rt and stirred for another 3 h. The reaction was quenched by the addition of aqueous 1 N HCl (0.6 mL) and the solution was diluted with H₂O (3 mL). The mixture was extracted with EtOAc (6 mL×3), and the combined organic phase was dried over anhydrous Na₂SO₄, filtered and concentrated under reduced pressure. PTLC (SiO₂, 1-10% MeOH—CH₂Cl₂ gradient) afforded the intermediate coupled bromide (39 mg, 62%, Compound A) as a white solid. ¹H NMR (CDCl₃, 400 MHz) δ 7.53 (s, 4H), 7.43 (br, 1H), 7.35 (br, 1H), 7.27-7.06 (m, 15H), 6.82 (br, 1H), 6.74 (br, 1H), 3.84-3.59 (m, 8H), 3.51 (td, J=6.7, 2.5 Hz, 1H), 3.39 (td, J=6.9, 2.4 Hz, 1H), 3.33-2.93 (m, 6H), 2.92-2.80 (m, 3H), 2.06-1.94 (m, 3H), 1.86-1.70 (m, 2H), 1.49-1.04 (m, 24H). HRMS (ESI-TOF) m/z calcd for C₅₉H₇₂BrN₆O₆ [M+H]⁺ 1039.4697, found 1039.4679.

A reaction solution of this material (16 mg, 0.015 mmol), N-acetyl glycine (3.5 mg, 0.030 mmol), NaI (9.0 mg, 0.060 mmol), and K₂CO₃ (4.1 mg, 0.030 mmol) in anhydrous DMF (0.2 mL) was stirred at 60° C. for overnight (about 18 h). The reaction solution was cooled to rt and quenched with the addition of H₂O (1 mL). The mixture was extracted with EtOAc (1.5 mL×3), and the combined organic phase was dried over anhydrous Na₂SO₄, filtered and concentrated under reduced pressure. PTLC (SiO₂, 1-10% MeOH—CH₂Cl₂ gradient) afforded Compound 40 (10 mg, 62%) as a white solid. ¹H NMR (CDCl₃, 400 MHz) δ 7.55-7.51 (m, 4H), 7.43-7.38 (m, 1H), 7.35-7.33 (m, 1H), 7.28-7.07 (m, 15H), 6.88-6.77 (m, 1H), 6.25-6.17 (m, 1H), 4.15-4.10 (m, 2H), 4.01-3.98 (m, 2H), 3.84-3.58 (m, 8H), 3.35-3.08 (m, 4H), 3.06-2.80 (m, 5H), 2.06-1.94 (m, 6H), 1.65-1.58 (m, 2H), 1.51-1.40 (m, 2H), 1.34-1.04 (m, 22H). HRMS (ESI-TOF) m/z calcd for C₆₃H₇₈N₇O₉ [M+H]⁺ 1076.5861, found 1076.5845.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)-carbamoyl)pyrrolidine-3-carboxamido)-dodecyl 2-(2,2,2-trifluoroacetamido)acetate (Compound 41)

A reaction solution of Compound 7 (19 mg, 0.020 mmol), 2-(2,2,2-trifluoroacetamido)acetic acid (3.4 mg, 0.020 mmol), DMAP (2.4 mg, 0.020 mmol), and EDCI·HCl (5.8 mg, 0.030 mmol) in anhydrous DMF (0.2 mL) and CH₂Cl₂ (0.2 mL) was stirred at 40° C. (or rt) for 8 h (or overnight; ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 41 (10 mg, 44%) as a white solid. ¹H NMR (CDCl₃, 400 MHz) δ 7.56-7.51 (m, 4H), 7.34 (br, 1H), 7.30 (br, 1H), 7.28-7.07 (m, 15H), 6.78 (br, 1H), 6.69 (br, 1H), 4.19-4.15 (m, 2H), 4.09-4.07 (m, 2H), 3.84-3.60 (m, 8H), 3.35-3.09 (m, 4H), 3.04-2.80 (m, 5H), 2.06-1.94 (m, 3H), 1.66-1.59 (m, 2H), 1.50-1.40 (m, 2H), 1.33-1.04 (m, 22H). ¹⁹F NMR (CDCl₃, 376 MHz) d −75.9 (d, J=13.9 Hz, 3F). HRMS (ESI-TOF) m/z calcd for C₆₃H₇₅F3N₇O₉[M+H]⁺ 1130.5578, found 1130.5551.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)carbamoyl)pyrrolidine-3-carboxamido)-dodecyl 2-pivalamidoacetate (Compound 42)

A reaction solution of intermediate compound A detailed above (38 mg, 0.037 mmol), 2-pivalamidoacetic acid (12 mg, 0.073 mmol), NaI (22 mg, 0.146 mmol), and K₂CO₃(10 mg, 0.073 mmol) in anhydrous DMF (0.3 mL) was stirred at 60° C. for overnight. The reaction solution was cooled to rt and quenched with the addition of H₂O (1 mL). The mixture was extracted with EtOAc (1.5 mL×3), and the combined organic phase was dried over anhydrous Na₂SO₄, filtered and concentrated under reduced pressure. PTLC (SiO₂, 1-10% MeOH—CH₂Cl₂ gradient) afforded Compound 42 (19 mg, 46%) as a white solid. ¹H NMR (CDCl₃, 400 MHz) δ 7.53 (s, 4H), 7.43 (br, 1H), 7.36-7.06 (m, 17H), 6.87-6.75 (m, 1H), 6.25-6.20 (m, 1H), 4.15-4.11 (m, 2H), 4.00-3.98 (m, 2H), 3.84-3.59 (m, 8H), 3.35-3.08 (m, 4H), 3.06-2.80 (m, 5H), 2.05-1.94 (m, 3H), 1.65-1.58 (m, 2H), 1.50-1.40 (m, 2H), 1.33-1.06 (m, 31H). HRMS (ESI-TOF) m/z calcd for C₆₆H₈₄N₇O₉ [M+H]⁺ 1118.6331, found 1118.6316.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)carbamoyl)pyrrolidine-3-carboxamido)dodecyl glycinate (Compound 43)

A solution of Compound 35 (diprovocim-X, 11 mg, 0.010 mmol) in 4 N HCl/1,4-dioxane (0.50 mL) was stirred at rt for 1 h. The solvent was removed and the residue was purified by HPLC using the gradient 0.07% TFA-water/CH₃CN (40%→70%) elution system to afford Compound 43 (11 mg, 96%) as a white solid. ¹H NMR (CD₃OD, 500 MHz) δ 7.62 (s, 4H), 7.27-7.20 (m, 5H), 7.17-7.06 (m, 10H), 4.23 (td, J=6.7, 2.7 Hz, 2H), 4.02-3.97 (m, 2H), 3.82 (s, 2H), 3.79-3.60 (m, 6H), 3.35-3.02 (m, 6H), 2.91-2.78 (m, 3H), 2.08-1.92 (m, 3H), 1.70-1.64 (m, 2H), 1.53-1.42 (m, 2H), 1.37-1.08 (m, 22H). HRMS (ESI-TOF) m/z calcd for C₆₁H₇₆N₇O₈ [M+H]⁺ 1034.5755, found 1034.5746.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclo-propyl)carbamoyl)pyrrolidine-3-carboxamido)-dodecyl 2-(dimethylamino)acetate (Compound 44)

A solution of N,N-dimethylglycine hydrochloride (2.0 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight; ca. 18 h). The reaction mixture was quenched by the addition of aqueous 1 N HCl (0.2 mL) and diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 44 (10 mg, 94%) as a white solid. ¹H NMR (CD₃OD, 400 MHz) δ 7.62 (s, 4H), 7.27-7.07 (m, 15H), 4.11 (td, J=6.7, 1.7 Hz, 2H), 4.03-3.97 (m, 2H), 3.79-3.60 (m, 6H), 3.35-3.01 (m, 8H), 2.91-2.78 (m, 3H), 2.34 (s, 6H), 2.09-1.91 (m, 3H), 1.67-1.59 (m, 2H), 1.55-1.42 (m, 2H), 1.36-1.07 (m, 22H). HRMS (ESI-TOF) m/z calcd for C₆₃H₈₀N₇O₈[M+H]⁺ 1062.6068, found 1062.6075.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclo-propyl)carbamoyl)pyrrolidine-3-carboxamido)dodecyl 2-hydroxyacetate (Compound 45

A solution of Compound 47 (10 mg, 0.0092 mmol) in TFA (0.15 mL) and anhydrous CH₂Cl₂ (0.15 mL) was stirred at rt for 8 h. The liquids were concentrated under reduced pressure. PTLC (SiO₂, 15% MeOH—CH₂Cl₂) afforded Compound 45 (6.5 mg, 68%) as a white solid. ¹H NMR (CDCl₃, 500 MHz) δ 7.56-7.52 (m, 4H), 7.27-7.07 (m, 15H), 4.19-4.15 (m, 2H), 4.14 (s, 2H), 3.81-3.61 (m, 8H), 3.32-3.11 (m, 4H), 3.05-2.96 (m, 2H), 2.90-2.81 (m, 3H), 2.05-1.96 (m, 3H), 1.65-1.60 (m, 2H), 1.49-1.40 (m, 2H), 1.33-1.05 (m, 22H). HRMS (ESI-TOF) m/z calcd for C₆₁H₇₅N₆O₉ [M+H]⁺ 1035.5596, found 1035.5593.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)-dodecyl 2-(benzyloxy)acetate (Compound 46)

A solution of 2-benzyloxyacetic acid (2.3 mg, 0.014 mmol) in anhydrous THF (0.14 mL) was treated with Et₃N (3.8 μL, 0.027 mmol) and 2,4,6-trichloro-benzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight, ca. 18 h). The reaction mixture was quenched by the addition of aqueous 1 N HCl (0.2 mL) and diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 46 (10 mg, 89%) as a white solid. ¹H NMR (CD₃OD, 400 MHz) δ 7.52 (s, 4H), 7.36-7.07 (m, 20H), 6.81-6.69 (m, 2H), 4.63 (s, 2H), 4.16-4.12 (m, 2H), 4.09 (s, 2H), 3.81-3.60 (m, 8H), 3.35-2.95 (m, 6H), 2.90-2.81 (m, 3H), 2.05-1.94 (m, 3H), 1.65-1.59 (m, 2H), 1.50-1.40 (m, 2H), 1.35-1.05 (m, 22H). HRMS (ESI-TOF) m/z calcd for C₆₈H₈₁N₆O₉ [M+H]⁺ 1125.6065, found 1125.6074.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)dodecyl 2-(tert-butoxy)acetate (Compound 47)

A reaction solution of 7 (14.7 mg, 0.015 mmol), 2-t-butyloxyacetic acid (2.0 mg, 0.015 mmol), DMAP (1.8 mg, 0.015 mmol), and EDCI·HCl (4.4 mg, 0.023 mmol) in anhydrous DMF (0.15 mL) and CH₂Cl₂ (0.15 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 47 (10 mg, 61%) as a white solid. ¹H NMR (CDCl₃, 500 MHz) δ 7.56-7.52 (m, 4H), 7.28-7.22 (m, 4H), 7.19-7.09 (m, 11H), 4.16-4.10 (m, 2H), 4.01 (s, 2H), 3.82-3.63 (m, 8H), 3.32-3.10 (m, 4H), 3.04-2.96 (m, 2H), 2.91-2.82 (m, 3H), 2.06-1.96 (m, 3H), 1.64-1.60 (m, 2H), 1.48-1.44 (m, 2H), 1.28-1.07 (m, 31H). HRMS (ESI-TOF) m/z calcd for C₆₅H₈₃N₆O₉ [M+H]⁺ 1091.6222, found 1091.6227.

### tert-Butyl (2-((12-((3S,4S)-1-(4-((3S,4S)-3,4-bis(((1S,2R)-2-phenylcyclopropyl)carbamoyl)-pyrrolidine-1-carbonyl)-benzoyl)-4-(((1S,2R)-2-phenylcyclopropyl)carbamoyl)pyrrolidine-3-carbox-amido)dodecyl)oxy)ethyl)carbamate (Compound 48)

A solution of Compound 2¹⁵ (63 mg, 0.080 mmol), tert-butyl (2-((12-aminododecyl)oxy)ethyl)-carbamate²³ (41 mg, 0.12 mmol), HOAt (13 mg, 0.096 mmol), and 2,6-lutidine (47 μL, 0.40 mmol) in anhydrous DMF (1.0 mL) was cooled to 0° C. and treated with EDCI·HCl (23 mg, 0.12 mmol) in one portion. The reaction solution was stirred at 0° C. for 30 min, warmed to rt and stirred for overnight (ca. 18 h). The reaction was quenched by the addition of aqueous 1 N HCl (1 mL) and the solution was diluted with H₂O (5 mL). The mixture was extracted with EtOAc (10 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 48 (63 mg, 70%) as a white solid. ¹H NMR (CDCl₃, 500 MHz) δ 7.54-7.50 (m, 4H), 7.49 (br, 1H), 7.41-7.34 (m, 2H), 7.25-7.05 (m, 15H), 6.88-6.80 (m, 1H), 4.90 (br, 1H), 3.82-3.59 (m, 8H), 3.46-3.38 (m, 4H), 3.33-3.07 (m, 6H), 3.04-2.94 (m, 2H), 2.90-2.81 (m, 3H), 2.04-1.94 (m, 3H), 1.55-1.52 (m, 2H), 1.48-1.40 (m, 11H), 1.30-1.05 (m, 22H). ¹³C NMR (CDCl₃, 150 MHz) δ 171.9, 171.8, 171.3, 170.5, 170.0, 168.6, 168.5, 168.4, 156.2, 140.4, 138.1, 138.0, 137.9, 137.8, 128.6, 127.6, 127.5, 126.5, 126.4, 126.3, 79.4, 71.4, 69.8, 53.6, 52.2, 48.9, 47.9, 47.8, 47.7, 47.6, 45.8, 45.6, 45.5, 45.3, 40.6, 40.0, 32.4, 32.3, 29.9, 29.8, 29.6, 29.5, 28.6, 27.1, 26.3, 24.9, 24.8, 24.7, 16.1, 15.8. HRMS (ESI-TOF) m/z calcd for C₆₆H₈₆N₇O₉ [M+H]⁺ 1120.6487, found 1120.6506.

### tert-Butyl 2-((12-((3S,4S)-1-(4-((3S,4S)-3,4-bis(((1S,2R)-2-phenylcyclopropyl)carbamoyl)-pyrrolidine-1-carbonyl)benzoyl)-4-(((1S,2R)-2-phenylcyclopropyl)carbamoyl)pyrrolidine-3-carboxamido)dodecyl)oxy)acetate (Compound 49)

A solution of 2¹⁵ (16 mg, 0.020 mmol), tert-butyl 2-((12-aminododecyl)oxy)acetate²³ (22 mg, 0.070 mmol), HOAt (3.3 mg, 0.024 mmol), and 2,6-lutidine (12 μL, 0.10 mmol) in anhydrous DMF (0.2 mL) was cooled to 0° C. and treated with EDCI·HCl (5.7 mg, 0.030 mmol) in one portion. The reaction solution was stirred at 0° C. for 30 min, warmed to rt and stirred for overnight (ca. 18 h). The reaction was quenched by the addition of aqueous 1 N HCl (0.1 mL) and the solution was diluted with H₂O (0.5 mL). The mixture was extracted with EtOAc (1 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 49 (7.0 mg, 32%) as a white solid. ¹H NMR (CDCl₃, 500 MHz) δ 7.56-7.52 (m, 4H), 7.28-7.08 (m, 15H), 3.95 (s, 2H), 3.84-3.61 (m, 8H), 3.49 (q, J=6.4 Hz, 2H), 3.34-3.08 (m, 4H), 3.06-2.95 (m, 2H), 2.92-2.83 (m, 3H), 2.07-1.96 (m, 3H), 1.64-1.56 (m, 2H), 1.51-1.43 (m, 11H), 1.36-1.07 (m, 22H). HRMS (ESI-TOF) m/z calcd for C₆₅H₈₃N₆O₉ [M+H]⁺ 1091.6222, found 1091.6202.

### Methyl 2-((((12-((3S,4S)-1-(4-((3S,4S)-3,4-bis-(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)dodecyl)oxy)-carbonyl)amino)acetate (Compound 50)

A solution of Compound 7 (9.8 mg, 0.010 mmol) in anhydrous CH₂Cl₂ (0.2 mL) was treated with anhydrous pyridine (3.2 μL, 0.040 mmol) and 4-nitrophenyl chloroformate (4.0 mg, 0.020 mmol) at rt. The reaction mixture was stirred at 30° C. for overnight and then diluted with EtOAc (1 mL). The organic layer was washed with aqueous 10% citric acid (1 mL) and saturated aqueous NaCl (1 mL), and dried over anhydrous Na₂SO₄, filtered and concentrated under reduced pressure. PTLC (SiO₂, 10% MeOH—CH₂Cl₂) afforded the activated carbonate (9.0 mg, 79%, Compound B) as a white solid. ¹H NMR (CDCl₃, 500 MHz) δ 8.27 (d, J=9.1 Hz, 2H), 7.56-7.52 (m, 4H), 7.37 (dd, J=9.1, 1.8 Hz, 2H), 7.27-7.08 (m, 15H), 4.27 (td, J=6.7, 2.6 Hz, 2H), 3.84-3.61 (m, 8H), 3.32-3.10 (m, 4H), 3.04-2.95 (m, 2H), 2.91-2.82 (m, 3H), 2.06-1.96 (m, 3H), 1.77-1.71 (m, 2H), 1.48-1.06 (m, 24H). HRMS (ESI-TOF) m/z calcd for C₆₆H₇₆N₇O₁₁ [M+H]⁺ 1142.5597, found 1142.5586.

A solution of this intermediate (0.01 mmol) and glycine methyl ester hydrochloride (2.5 mg, 0.020 mmol) in anhydrous CH₂Cl₂ (0.2 mL) and DMF (0.2 mL) was treated with Et₃N (2.8 μL, 0.020 mmol) at rt. The reaction mixture was stirred at 45° C. for overnight (ca. 18 h) and then cooled to rt. The reaction was quenched by the addition of aqueous 1 N HCl (0.2 mL) and the solution was diluted with H₂O (1 mL). The mixture was extracted with EtOAc (1 mL×3), and the combined organic phase was dried over anhydrous Na₂SO₄, filtered and concentrated under reduced pressure. Flash chromatography (SiO₂, 1-10% MeOH—CH₂Cl₂ gradient) afforded Compound 50 (5.6 mg, 51%) as a white solid. ¹H NMR (CD₃OD, 400 MHz) δ 7.62 (s, 4H), 7.27-7.07 (m, 15H), 4.05-3.97 (m, 4H), 3.83-3.60 (m, 11H), 3.35-3.02 (m, 6H), 2.91-2.78 (m, 3H), 2.09-1.91 (m, 3H), 1.60-1.42 (m, 4H), 1.36-1.07 (m, 22H). HRMS (ESI-TOF) m/z calcd for C₆₃H₇₈N₇O₁₀ [M+H]⁺ 1092.5810, found 1092.5802.

### (3S,4S)-1-(4-((3S,4S)-3,4-Bis(((1S,2R)-2-phenylcyclo-propyl)carbamoyl)pyrrolidine-1-carbonyl)-benzoyl)-N³-(12-((4-nitrobenzyl)oxy) dodecyl)-N⁴-((1S,2R)-2-phenyl-cyclopropyl)pyrrolidine-3,4-dicarboxamide (Compound 51)

A solution of Compound 2¹⁵ (32 mg, 0.040 mmol), 12-((4-nitrobenzyl)oxy)dodecan-1-amine hydrogen chloride²³ (22 mg, 0.060 mmol), HOAt (6.5 mg, 0.048 mmol), and 2,6-lutidine (23 μL, 0.20 mmol) in anhydrous DMF (0.8 mL) was cooled to 0° C. and treated with EDCI·HCl (12 mg, 0.060 mmol) in one portion. The reaction solution was stirred at 0° C. for 30 min, warmed to rt and stirred for another 4 h. The reaction was quenched by the addition of aqueous 1 N HCl (1.0 mL) and the solution was diluted with H₂O (4 mL). The mixture was extracted with EtOAc (8 mL×3), and the combined organic phase was dried over anhydrous Na₂SO₄, filtered and concentrated under reduced pressure. PTLC (SiO₂, 10% MeOH—CH₂Cl₂) afforded Compound 51 (36 mg, 81%) as a white solid. ¹H NMR (CDCl₃, 500 MHz) δ 8.19 (d, J=7.2 Hz, 2H), 7.56-7.52 (m, 4H), 7.49 (d, J=7.8 Hz, 2H), 7.27-7.22 (m, 5H), 7.18-7.08 (m, 10H), 4.58 (s, 2H), 3.85-3.62 (m, 8H), 3.50 (td, J=6.7, 3.1 Hz, 2H), 3.32-3.08 (m, 4H), 3.04-2.94 (m, 2H), 2.91-2.81 (m, 3H), 2.06-1.95 (m, 3H), 1.66-1.59 (m, 2H), 1.48-1.42 (m, 2H), 1.38-1.34 (m, 2H), 1.28-1.05 (m, 20H). HRMS (ESI-TOF) m/z calcd for C₆₆H₇₈N₇O₉ [M+H]⁺ 1082.6114, found 1082.6105.

### (3S,4S)—N³-(12-((4-Aminobenzyl)oxy)dodecyl)-1-(4-((3S,4S)-3,4-bis(((1S,2R)-2-phenyl-cyclopropyl)carbamoyl)pyrrolidine-1-carbonyl)-benzoyl)-N⁴-((1S,2R)-2-phenylcyclopropyl)pyrrolidine-3,4-dicarboxamide (Compound 52)

Step 1: A solution of Compound 2 (53 mg, 0.067 mmol), tert-butyl (4-(((12-aminododecyl)oxy)-methyl)phenyl)carbamate²³ (41 mg, 0.10 mmol), HOAt (11 mg, 0.080 mmol), and 2,6-lutidine (38 μL, 0.33 mmol) in anhydrous DMF (1.2 mL) was cooled to 0° C. and treated with EDCI·HCl (19 mg, 0.10 mmol) in one portion. The reaction solution was stirred at 0° C. for 30 min, warmed to rt and stirred for another 8 h. The reaction was quenched by the addition of aqueous 1 N HCl (2.0 mL) and the solution diluted with H₂O (8 mL). The mixture was extracted with EtOAc (16 mL×3), and the combined organic phase was dried over anhydrous Na₂SO₄, filtered and concentrated under reduced pressure. PTLC (SiO₂, 10% MeOH—CH₂Cl₂) afforded the NHBoc derivative of Compound 52 (51 mg, 64%) as a white solid. ¹H NMR (CDCl₃, 500 MHz) δ 7.55-7.51 (m, 4H), 7.32 (d, J=8.2 Hz, 2H), 7.27-7.06 (m, 17H), 4.42 (s, 2H), 3.83-3.60 (m, 8H), 3.41 (td, J=6.6, 3.2 Hz, 2H), 3.32-3.08 (m, 4H), 3.04-2.93 (m, 2H), 2.91-2.82 (m, 3H), 2.05-1.95 (m, 3H), 1.60-1.53 (m, 2H), 1.51-1.41 (m, 11H), 1.32-1.07 (m, 22H). HRMS (ESI-TOF) m/z calcd for C₇₁H₈₈N₇O₉ [M+H]⁺ 1182.6638, found 1182.6638.

Step 2: A solution of this material (24 mg, 0.020 mmol) in 4 N HCl/1,4-dioxane (0.30 mL) was stirred at rt for 1 h. The solvent was removed and the residue was purified by PTLC (SiO₂, 10% MeOH—CH₂Cl₂) afforded Compound 52 (10 mg, 46%) as a white solid. ¹H NMR (CDCl₃, 500 MHz) δ 7.53 (s, 4H), 7.27-7.05 (m, 17H), 6.72 (s, 2H), 4.37 (s, 2H), 3.84-3.61 (m, 8H), 3.43-3.38 (m, 2H), 3.32-3.00 (m, 6H), 2.91-2.80 (m, 3H), 2.06-1.95 (m, 3H), 1.60-1.53 (m, 2H), 1.50-1.40 (m, 2H), 1.33-1.07 (m, 22H). HRMS (ESI-TOF) m/z calcd for C₆₆H₈₀N₇O₇[M+H]⁺ 1082.6114, found 1082.6116.

### 12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)dodecyl 2-(methyl(prop-2-yn-1-yl)amino)acetate (Compound 53)

A solution of 2-(methyl(prop-2-yn-1-yl)amino)trifluoroacetic acid²⁸ (3.4 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 53 (5.4 mg, 50%) as a white solid. ¹H NMR (CDCl₃, 400 MHz) δ 7.50 (m, 4H), 7.26-7.04 (m, 15H), 4.14-4.10 (m, 2H), 3.91-3.74 (m, 4H), 3.71-3.66 (m, 3H), 3.63 (s, 2H), 3.49 (s, 1H), 3.45 (s, 2H), 3.32-3.06 (m, 6H), 2.96-2.80 (m, 3H), 2.53 (s, 3H), 2.32 (s, 1H), 2.05-1.93 (m, 3H), 1.65-1.59 (m, 2H), 1.47-1.38 (m, 2H), 1.33-1.04 (m, 22H). HRMS (ESI-TOF) m/z calcd for C₆₅H₈₀N₇O₈[M+H]⁺ 1086.6063, found 1086.6069.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)dodecyl 2-(prop-2-yn-1-yloxy)acetate (Compound 54)

A solution of 2-(prop-2-yn-1-yloxy)acetic acid²⁹ (1.6 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 54 (10 mg, 93%) as a white solid. ¹H NMR (CD₃OD, 400 MHz) δ 7.62 (s, 4H), 7.27-7.06 (m, 15H), 4.27 (d, J=2.3 Hz, 2H), 4.18 (s, 2H), 4.14 (t, J=6.7 Hz, 2H), 4.03-3.96 (m, 2H), 3.79-3.60 (m, 6H), 3.35-3.01 (m, 6H), 2.92-2.78 (m, 4H), 2.09-1.91 (m, 3H), 1.67-1.59 (m, 2H), 1.54-1.41 (m, 2H), 1.37-1.07 (m, 22H). HRMS (ESI-TOF) m/z calcd for C₆₄H₇₇N₆O₉ [M+H]⁺ 1073.5746, found 1073.5739.

### 12-((3S,4S)-1-(4-((3S,4S)-3,4-Bis(((1S,2R)-2-phenyl-cyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2,2-dimethyl-4-oxo-3,8,11,14,17-pentaoxa-5-azaicosan-20-oate (Compound 55)

A solution of 2,2-dimethyl-4-oxo-3,8,11,14,17-pentaoxa-5-azaicosan-20-oic acid (5.1 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 55 (9.8 mg, 74%) as a white solid. ¹H NMR (CD₃OD, 400 MHz) δ 7.62 (s, 4H), 7.27-7.07 (m, 15H), 4.07 (t, J=6.6 Hz, 2H), 4.03-3.96 (m, 2H), 3.79-3.60 (m, 20H), 3.50 (t, J=5.6 Hz, 2H), 3.35-3.01 (m, 8H), 2.91-2.78 (m, 3H), 2.56 (t, J=6.2 Hz, 2H), 2.09-1.91 (m, 3H), 1.65-1.49 (m, 4H), 1.43 (s, 9H), 1.36-1.07 (m, 22H). HRMS (ESI-TOF) m/z calcd for C₇₅H₁₀₂N₇O₁₄ [M+H]⁺ 1324.7485, found 1324.7457.

### tert-Butyl 1-((3S,4S)-1-(4-((3S,4S)-3,4-bis(((1S,2R)-2-phenylcyclopropyl)carbamoyl)pyrrolidine-1carbonyl)benzoyl)-4-(((1S,2R)-2-phenylcyclopropyl)-carbamoyl)pyrrolidin-3-yl)-1,14-dioxo-18,21,24-trioxa-2,15-diazaheptacosan-27-oate (Compound 56)

Step 1: A solution of Compound 3 (36 mg, 0.036 mmol) in THF (0.40 mL), MeOH (0.10 mL), and H₂O (0.10 mL) was treated with LiOH·H₂O (6.0 mg, 0.144 mmol) at 0° C., and stirred at rt for 2 h. The reaction solution was diluted with H₂O (1 mL) and acidified with aqueous 1 N HCl to pH 3. The mixture was extracted with EtOAc (2 mL×3), and the combined organic phase was dried over anhydrous Na₂SO₄, filtered, and concentrated in vacuo to afford the carboxylic acid Compound 11 (35 mg, 98%) as a white solid, which was used without further purification.

Step 2: A solution of Compound 11 (9.9 mg, 0.010 mmol), H₂N(CH₂CH₂O)₃CH₂CH₂CO₂ tBu (4.2 mg, 0.015 mmol), HOAt (1.6 mg, 0.012 mmol), and 2,6-lutidine (5.8 μL, 0.050 mmol) in anhydrous DMF (0.2 mL) was cooled to 0° C. and treated with EDCI·HCl (2.9 mg, 0.015 mmol) in one portion. The reaction solution was stirred at 0° C. for 30 min, warmed to rt and stirred for another 3 h. The reaction was quenched by the addition of aqueous 1 N HCl (0.3 mL) and the solution was diluted with H₂O (1 mL). The mixture was extracted with EtOAc (2 mL×3), and the combined organic phase was dried over anhydrous Na₂SO₄, filtered and concentrated under reduced pressure. PTLC (SiO₂, 10% MeOH—CH₂Cl₂) afforded Compound 56 (9.0 mg, 72%) as a white solid. ¹H NMR (CD₃OD, 400 MHz) δ 7.62 (s, 4H), 7.27-7.07 (m, 15H), 4.03-3.97 (m, 2H), 3.79-3.58 (m, 16H), 3.52 (t, J=5.5 Hz, 2H), 3.35-3.01 (m, 8H), 2.91-2.78 (m, 3H), 2.47 (t, J=6.3 Hz, 2H), 2.18 (t, J=7.6 Hz, 2H), 2.09-1.91 (m, 3H), 1.62-1.41 (m, 13H), 1.34-1.08 (m, 22H). HRMS (ESI-TOF) m/z calcd for C₇₂H₉₆N₇O₁₂ [M+H]⁺ 1250.7117, found 1250.7095.

### (S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-((tert-butoxycarbonyl)amino)propanoate (Compound 57)

A solution of BocNH-L-Ala-OH (2.6 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred at 40° C. (or rt) for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 57 (8.6 mg, 75%) as a white solid. ¹H NMR (CDCl₃, 400 MHz) δ 7.56-7.52 (m, 4H), 7.35 (br, 1H), 7.28-7.08 (m, 15H), 6.75 (br, 1H), 6.65 (br, 1H), 5.09 (br, 1H), 4.33-4.25 (m, 1H), 4.14-4.08 (m, 2H), 3.85-3.61 (m, 8H), 3.35-3.08 (m, 4H), 3.05-2.82 (m, 5H), 2.04-1.96 (m, 3H), 1.65-1.58 (m, 2H), 1.51-1.41 (m, 11H), 1.37 (d, J=7.2 Hz, 3H), 1.32-1.05 (m, 22H). HRMS (ESI-TOF) m/z calcd for C₆₇H₈₆N₇O₁₀ [M+H]⁺ 1148.6436, found 1148.6454.

### 12-((3S,4S)-1-(4-((3S,4S)-3,4-Bis(((1S,2R)-2-phenyl-cyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl (tert-butoxycarbonyl)-D-alaninate (Compound 58)

A reaction solution of Compound 7 (9.8 mg, 0.010 mmol), BocNH-D-Ala-OH (1.9 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 58 (7.7 mg, 67%) as a white solid. ¹H NMR (CDCl₃, 500 MHz) δ 7.56-7.52 (m, 4H), 7.27-7.22 (m, 5H), 7.18-7.07 (m, 10H), 5.10 (br, 1H), 4.32-4.25 (m, 1H), 4.16-4.06 (m, 2H), 3.81-3.62 (m, 8H), 3.32-3.08 (m, 4H), 3.06-2.94 (m, 2H), 2.91-2.82 (m, 3H), 2.05-1.95 (m, 3H), 1.65-1.58 (m, 2H), 1.50-1.44 (m, 11H), 1.37 (d, J=7.1 Hz, 3H), 1.31-1.06 (m, 22H). HRMS (ESI-TOF) m/z calcd for C₆₇H₈₆N₇O₁₀ [M+H]⁺ 1148.6436, found 1148.6407.

### (S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-((tert-butoxycarbonyl)amino)-3-methylbutanoate (Compound 59)

A solution of Compound 7 (9.8 mg, 0.010 mmol), BocNH-L-Val-OH (2.2 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 59 (5.8 mg, 49%) as a white solid. ¹H NMR (CDCl₃, 400 MHz) δ 7.56-7.52 (m, 4H), 7.31 (br, 1H), 7.28-7.08 (m, 15H), 6.70 (br, 1H), 6.60 (br, 1H), 5.07-5.01 (m, 1H), 4.23-4.17 (m, 1H), 4.15-4.04 (m, 2H), 3.85-3.62 (m, 8H), 3.32-3.08 (m, 4H), 3.06-2.80 (m, 5H), 2.17-2.08 (m, 1H), 2.07-1.95 (m, 3H), 1.63-1.56 (m, 2H), 1.51-1.38 (m, 11H), 1.35-1.04 (m, 22H), 0.95 (d, J=6.8 Hz, 3H), 0.88 (d, J=6.8 Hz, 3H). HRMS (ESI-TOF) m/z calcd for C₆₉H₉₀N₇O₁₀ [M+H]⁺ 1176.6749, found 1176.6725.

### (S)-2-(12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl) 1-tert-butyl pyrrolidine-1,2-dicarboxylate (Compound 60)

A reaction solution of Compound 7 (9.8 mg, 0.010 mmol), Boc-L-Pro-OH (2.1 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 60 (7.4 mg, 63%) as a white solid. ¹H NMR (CDCl₃, 400 MHz) δ 7.56-7.51 (m, 4H), 7.33 (br, 1H), 7.28-7.08 (m, 15H), 6.74 (br, 1H), 6.65 (br, 1H), 4.32-4.27 (m, 1H), 4.21 (dd, J=8.6, 3.8 Hz, 1H), 4.15-4.01 (m, 2H), 3.85-3.59 (m, 8H), 3.57-3.34 (m, 2H), 3.33-3.08 (m, 4H), 3.05-2.81 (m, 5H), 2.26-2.11 (m, 1H), 2.07-1.80 (m, 6H), 1.64-1.56 (m, 2H), 1.51-1.38 (m, 11H), 1.34-1.04 (m, 22H). HRMS (ESI-TOF) m/z calcd for C₆₉H₈₈N₇O₁₀ [M+H]⁺ 1174.6593, found 1174.6575.

### (2S,3S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-((tert-butoxy-carbonyl)amino)-3-methylpentanoate (Compound 61)

A solution of BocNH-L-IIe-OH (2.3 mg, 0.010 mmol) in anhydrous THF (0.14 mL) was treated with Et₃N (1.4 μL, 0.010 mmol) and 2,4,6-trichlorobenzoyl chloride (1.7 μL, 0.011 mmol) at 0° C. The reaction mixture was stirred at 0° C. for 2 h, before being transferred to a suspension of Compound 7 (9.8 mg, 0.010 mmol) and DMAP (0.61 mg, 0.0050 mmol) in anhydrous THF (0.20 mL) at 0° C. The resulting reaction mixture was warmed to rt and stirred at 40° C. (or rt) for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 61 (6.6 mg, 55%) as a white solid. ¹H NMR (CDCl₃, 400 MHz) δ 7.56-7.52 (m, 4H), 7.32 (br, 1H), 7.28-7.08 (m, 15H), 6.71 (br, 1H), 6.61 (br, 1H), 5.06 (d, J=9.0 Hz, 1H), 4.28-4.21 (m, 1H), 4.16-4.03 (m, 2H), 3.85-3.62 (m, 8H), 3.33-3.08 (m, 4H), 3.05-2.81 (m, 5H), 2.04-1.95 (m, 3H), 1.90-1.79 (m, 1H), 1.65-1.57 (m, 2H), 1.52-1.37 (m, 11H), 1.34-1.05 (m, 24H), 0.92-0.88 (m, 6H). HRMS (ESI-TOF) m/z calcd for C₇₀H₉₂N₇O₁₀ [M+H]⁺ 1190.6906, found 1190.6896.

### 12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 1-((tert-butoxy-carbonyl)amino)cyclopropanecarboxylate (Compound 62)

A solution of 1-(N-Boc-amino)cyclopropane-carboxylic acid (2.8 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred at 40° C. (or rt) for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 62 (9.0 mg, 78%) as a white solid. ¹H NMR (CDCl₃, 400 MHz) δ 7.54 (s, 4H), 7.31 (br, 1H), 7.28-7.08 (m, 15H), 4.08-4.04 (m, 2H), 3.82-3.62 (m, 8H), 3.30-3.12 (m, 4H), 3.07-2.81 (m, 5H), 2.04-1.96 (m, 3H), 1.63-1.54 (m, 2H), 1.52-1.40 (m, 11H), 1.37-1.05 (m, 26H). HRMS (ESI-TOF) m/z calcd for C₆₈H₈₆N₇O₁₀ [M+H]⁺ 1060.6436, found 1060.6449.

### (S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-((tert-butoxy-carbonyl)amino)-3-phenylpropanoate (Compound 63)

A solution of BocNH-L-Phe-OH (3.7 mg, 0.014 mmol) in anhydrous THF (0.14 mL) was treated with Et₃N (3.8 μL, 0.027 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred at 40° C. (or rt) for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 63 (9.8 mg, 80%) as a white solid. ¹H NMR (CDCl₃, 400 MHz) δ 7.56-7.52 (m, 4H), 7.33 (br, 1H), 7.29-7.08 (m, 20H), 6.72 (br, 1H), 6.61 (br, 1H), 5.03-4.97 (m, 1H), 4.59-4.52 (m, 1H), 4.09-4.05 (m, 2H), 3.85-3.62 (m, 8H), 3.33-3.16 (m, 4H), 3.15-2.81 (m, 7H), 2.06-1.95 (m, 3H), 1.61-1.53 (m, 2H), 1.51-1.35 (m, 11H), 1.31-1.05 (m, 22H). HRMS (ESI-TOF) m/z calcd for C₇₃H₉₀N₇O₁₀ [M+H]⁺ 1224.6749, found 1224.6765.

### (S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)dodecyl 3-(benzyloxy)-2-((tert-butoxycarbonyl)amino)propanoate (Compound 64)

A reaction solution of Compound 7 (9.8 mg, 0.010 mmol), BocNH-L-Ser(OBn)-OH (2.9 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 64 (8.5 mg, 68%) as a white solid. ¹H NMR (CDCl₃, 400 MHz) δ 7.56-7.51 (m, 4H), 7.34-7.08 (m, 20H), 6.71 (br, 1H), 6.62 (br, 1H), 5.41 (d, J=8.8 Hz, 1H), 4.55-4.46 (m, 2H), 4.44-4.39 (m, 1H), 4.15-4.09 (m, 2H), 3.88-3.61 (m, 10H), 3.33-3.08 (m, 4H), 3.06-2.81 (m, 5H), 2.06-1.95 (m, 3H), 1.62-1.54 (m, 2H), 1.51-1.37 (m, 11H), 1.33-1.04 (m, 22H). HRMS (ESI-TOF) m/z calcd for C₇₄H₉₂N₇O₁₁ [M+H]⁺ 1254.6855, found 1254.6851.

### (R)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)dodecyl 3-(benzylthio)-2-((tert-butoxycarbonyl)amino)propanoate (Compound 65)

A reaction solution of Compound 7 (9.8 mg, 0.010 mmol), BocNH-L-Cys(SBn)-OH (3.1 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 65 (8.7 mg, 68%) as a white solid. ¹H NMR (CDCl₃, 400 MHz) δ 7.56-7.52 (m, 4H), 7.32 (br, 1H), 7.30-7.08 (m, 20H), 6.71 (br, 1H), 6.61 (br, 1H), 5.33-5.28 (m, 1H), 4.54-4.47 (m, 1H), 4.13-4.09 (m, 2H), 3.85-3.59 (m, 10H), 3.33-3.08 (m, 4H), 3.05-2.76 (m, 7H), 2.06-1.95 (m, 3H), 1.63-1.56 (m, 2H), 1.51-1.36 (m, 11H), 1.33-1.04 (m, 22H). HRMS (ESI-TOF) m/z calcd for C₇₄H₉₂N₇O₁₀S [M+H]⁺ 1270.6626, found 1270.6611.

### (S)-5-Benzyl 1-(12-((3S,4S)-1-(4-((3S,4S)-3,4-bis-(((1S,2R)-2-phenylcyclopropyl)carbamoyl)-pyrrolidine-1-carbonyl)-benzoyl)-4-(((1S,2R)-2-phenylcyclopropyl)-carbamoyl)pyrrolidine-3-carboxamido)dodecyl) 2-((tert-butoxycarbonyl)-amino)pentanedioate (Compound 66)

A reaction solution of Compound 7 (9.8 mg, 0.010 mmol), BocNH-L-Glu(OBn)-OH (3.4 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 66 (8.8 mg, 68%) as a white solid. ¹H NMR (CDCl₃, 400 MHz) δ 7.56-7.52 (m, 4H), 7.38-7.30 (m, 5H), 7.28-7.08 (m, 15H), 6.70 (br, 1H), 6.60 (br, 1H), 5.19-5.11 (br, 1H), 5.11 (s, 2H), 4.36-4.27 (m, 1H), 4.12-4.08 (m, 2H), 3.85-3.62 (m, 8H), 3.32-3.08 (m, 4H), 3.05-2.81 (m, 5H), 2.52-2.31 (m, 2H), 2.25-2.12 (m, 1H), 2.07-1.90 (m, 4H), 1.63-1.56 (m, 2H), 1.51-1.37 (m, 11H), 1.33-1.05 (m, 22H). HRMS (ESI-TOF) m/z calcd for C₇₆H₉₄N₇O₁₂ [M+H]⁺ 1296.6960, found 1296.6932.

### (S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-((tert-butoxy-carbonyl)amino)-4-(methylthio)butanoate (Compound 67)

A reaction solution of Compound 7 (9.8 mg, 0.010 mmol), BocNH-L-Met-OH (2.5 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 67 (7.0 mg, 58%) as a white solid. ¹H NMR (CDCl₃, 400 MHz) δ 7.56-7.52 (m, 4H), 7.34 (br, 1H), 7.28-7.08 (m, 15H), 6.74 (br, 1H), 6.63 (br, 1H), 5.22-5.13 (m, 1H), 4.43-4.35 (m, 1H), 4.14-4.10 (m, 2H), 3.85-3.62 (m, 8H), 3.33-3.09 (m, 4H), 3.05-2.81 (m, 5H), 2.53 (t, J=7.4 Hz, 2H), 2.18-1.87 (m, 8H), 1.65-1.56 (m, 2H), 1.53-1.38 (m, 11H), 1.35-1.04 (m, 22H). HRMS (ESI-TOF) m/z calcd for C₆₉H₉₀N₇O₁₀S [M+H]⁺ 1208.6470, found 1208.6461.

### (S)-12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)carbamoyl)pyrrolidine-3-carboxamido)-dodecyl 2-((tert-butoxy-carbonyl)amino)-3-(1-tosyl-1H-imidazol-4-yl)propanoate (Compound 68)

A reaction solution of Compound 7 (15 mg, 0.015 mmol), BocNH-L-His(Tos)-OH (6.1 mg, 0.015 mmol), DMAP (1.8 mg, 0.015 mmol), and EDCI·HCl (4.4 mg, 0.023 mmol) in anhydrous DMF (0.15 mL) and CH₂Cl₂ (0.15 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 68 (10 mg, 49%) as a white solid. ¹H NMR (CDCl₃, 400 MHz) δ 7.90 (s, 1H), 7.78 (d, J=8.1 Hz, 2H), 7.56-7.51 (m, 4H), 7.33 (d, J=7.7 Hz, 3H), 7.28-7.05 (m, 15H), 6.73 (br, 1H), 6.63 (br, 1H), 5.56 (d, J=8.4 Hz, 1H), 4.55-4.48 (m, 1H), 4.02-3.99 (m, 2H), 3.85-3.62 (m, 8H), 3.35-3.08 (m, 4H), 3.06-2.81 (m, 7H), 2.42 (s, 3H), 2.05-1.95 (m, 3H), 1.57-1.36 (m, 13H), 1.30-1.05 (m, 22H). HRMS (ESI-TOF) m/z calcd for C₇₇H₉₄N₉O₁₂S [M+H]⁺ 1368.6743, found 1368.6766.

### (S)-12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 6-((((9H-fluoren-9-yl)methoxy)-carbonyl)amino)-2-((tert-butoxy-carbonyl)amino)-hexanoate (Compound 69)

A solution of BocNH-L-Lys(Fmoc)-OH (6.6 mg, 0.014 mmol) in anhydrous THF (0.14 mL) was treated with Et₃N (1.4 μL, 0.010 mmol) and 2,4,6-trichlorobenzoyl chloride (3.4 μL, 0.022 mmol) at 0° C. The reaction mixture was stirred at 0° C. for 2 h, before being transferred to a suspension of Compound 7 (9.8 mg, 0.010 mmol) and DMAP (1.2 mg, 0.010 mmol) in anhydrous THF (0.20 mL) at 0° C. The resulting reaction mixture was warmed to rt and stirred at 40° C. (or rt) for 8 h (or overnight, ca. 18 h). The reaction was quenched by the addition of aqueous 1 N HCl (0.2 mL) and the solution was diluted with H₂O (1 mL). The mixture was extracted with EtOAc (1.5 mL×4), and the combined organic phase was dried over anhydrous Na₂SO₄, filtered and concentrated under reduced pressure. PTLC (SiO₂, 10% MeOH—CH₂Cl₂) afforded Compound 69 (9.9 mg, 69%) as a white solid. ¹H NMR (CDCl₃, 400 MHz) δ 7.76 (d, J=7.5 Hz, 2H), 7.58 (d, J=7.5 Hz, 2H), 7.53 (s, 4H), 7.41-7.07 (m, 19H), 5.12 (br, 1H), 4.94 (br, 1H), 4.48-4.33 (m, 2H), 4.31-4.17 (m, 2H), 4.13-4.06 (m, 2H), 3.81-3.61 (m, 8H), 3.33-2.94 (m, 8H), 2.92-2.80 (m, 3H), 2.06-1.94 (m, 3H), 1.91-1.69 (m, 2H), 1.67-1.57 (m, 2H), 1.52-1.35 (m, 13H), 1.31-1.04 (m, 24H). HRMS (ESI-TOF) m/z calcd for C₈₅H₁₀₃N₈O₁₂ [M+H]⁺ 1427.7695, found 1427.7743.

### 12-((3S,4S)-1-(4-((3S,4S)-3,4-Bis(((1S,2R)-2-phenyl-cyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 2-((tert-butoxycarbonyl)(methyl)amino)acetate (Compound 70)

A reaction solution of Compound 7 (9.8 mg, 0.010 mmol), Boc-Sar-OH (1.9 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 70 (8.3 mg, 72%) as a white solid. ¹H NMR (CDCl₃, 400 MHz) δ 7.56-7.51 (m, 4H), 7.34 (br, 1H), 7.28-7.08 (m, 15H), 6.73 (br, 1H), 6.64 (br, 1H), 4.14-4.08 (m, 2H), 3.96 (d, J=2.3 Hz, 1H), 3.88 (s, 1H), 3.82-3.61 (m, 8H), 3.30-3.09 (m, 4H), 3.04-2.82 (m, 8H), 2.06-1.95 (m, 3H), 1.64-1.56 (m, 2H), 1.49-1.42 (m, 11H), 1.27-1.04 (m, 22H). HRMS (ESI-TOF) m/z calcd for C₆₇H₈₆N₇O₁₀ [M+H]⁺ 1148.6436, found 1148.6406.

### 1-(12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl) 2-tert-butyl hydrazine-1,2-dicarboxylate (Compound 71)

A reaction solution of the intermediate activated carbonate prepared by treatment of Compound 7 with 4-nitrophenyl chloroformate as detailed earlier (compound B, 19 mg, 0.017 mmol), tert-butyl carbazate (4.5 mg, 0.034 mmol), and Et₃N (4.7 μL, 0.034 mmol) in anhydrous DMF (0.2 mL) and CH₂Cl₂ (0.2 mL) was stirred at 45° C. overnight (ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 71 (12 mg, 62%) as a white solid. ¹H NMR (CDCl₃, 400 MHz) δ 7.53 (s, 4H), 7.47 (br, 1H), 7.45 (br, 1H), 7.39 (br, 1H), 7.33 (br, 1H), 7.27-7.06 (m, 15H), 6.98 (br, 1H), 6.90 (br, 1H), 4.12-4.08 (m, 2H), 3.85-3.59 (m, 8H), 3.34-2.97 (m, 6H), 2.93-2.80 (m, 3H), 2.06-1.95 (m, 3H), 1.63-1.55 (m, 2H), 1.52-1.37 (m, 11H), 1.33-1.06 (m, 22H). HRMS (ESI-TOF) m/z calcd for C₆₅H₈₃N₈O₁₀ [M+H]⁺ 1135.6232, found 1135.6251.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)carbamoyl)pyrrolidine-3-carboxamido)-dodecyl 2-(2-((tert-butoxycarbonyl)amino)acetamido)acetate (Compound 72)

A reaction solution of Compound 7 (9.8 mg, 0.010 mmol), BocNH-Gly-Gly-OH (2.3 mg, 0.010 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 72 (5.4 mg, 45%) as a white solid. ¹H NMR (CDCl₃, 400 MHz) δ 7.54 (s, 4H), 7.31 (br, 1H), 7.28-7.08 (m, 15H), 6.91 (br, 1H), 6.81 (br, 1H), 6.72 (br, 1H), 5.36 (br, 1H), 4.15-4.10 (m, 2H), 4.03-3.97 (m, 2H), 3.87-3.62 (m, 10H), 3.33-3.12 (m, 4H), 3.10-2.81 (m, 5H), 2.08-1.95 (m, 3H), 1.68-1.57 (m, 4H), 1.45 (s, 9H), 1.33-1.06 (m, 22H). HRMS (ESI-TOF) m/z calcd for C₆₈H₈₇N₈O₁₁ [M+H]⁺ 1191.6494, found 1191.6470.

### 12-((3S,4S)-1-(4-((3S,4S)-3,4-Bis(((1S,2R)-2-phenyl-cyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl 4-((tert-butoxycarbonyl)amino)benzoate (Compound 73)

A reaction solution of Compound 7 (9.8 mg, 0.010 mmol), 4-((tert-butoxycarbonyl)amino)benzoic acid (3.6 mg, 0.015 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 73 (6.7 mg, 56%) as a white solid. ¹H NMR (CDCl₃, 400 MHz) δ 7.95 (dd, J=8.9, 2.5 Hz, 2H), 7.55-7.50 (m, 4H), 7.42 (d, J=8.5 Hz, 2H), 7.34 (br, 1H), 7.28-7.08 (m, 15H), 6.97 (br, 1H), 6.76 (br, 1H), 6.65 (br, 1H), 4.29-4.25 (m, 2H), 3.85-3.61 (m, 8H), 3.33-3.08 (m, 4H), 3.06-2.81 (m, 5H), 2.06-1.96 (m, 3H), 1.77-1.70 (m, 2H), 1.52 (s, 9H), 1.47-1.38 (m, 2H), 1.32-1.06 (m, 22H). HRMS (ESI-TOF) m/z calcd for C₇₁H₈₆N₇O₁₀ [M+H]⁺ 1196.6436, found 1196.6453.

### 12-((3S,4S)-1-(4-((3S,4S)-3,4-Bis(((1S,2R)-2-phenyl-cyclopropyl)carbamoyl)pyrrolidine-1-carbonyl)benzoyl)-4-(((1S,2R)-2-phenylcyclopropyl)carbamoyl)-pyrrolidine-3-carboxamido)-dodecyl 4-aminobenzoate (Compound 74)

A reaction solution of Compound 73 (9.3 mg, 0.0078 mmol) in 4 N HCl/1,4-dioxane (0.2 mL) was stirred at rt for 2 h. The reaction was quenched by the addition of H₂O (1 mL) and the solution was adjusted to pH 7 with the addition of saturated aqueous NaHCO₃. The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 74 (6.3 mg, 74%) as a white solid. ¹H NMR (CD₃OD, 400 MHz) δ 7.72 (d, J=8.4 Hz, 2H), 7.61 (s, 4H), 7.27-7.05 (m, 15H), 6.63 (d, J=8.4 Hz, 2H), 4.21 (t, J=6.6 Hz, 2H), 4.03-3.96 (m, 2H), 3.79-3.60 (m, 6H), 3.34-3.01 (m, 6H), 2.91-2.77 (m, 3H), 2.09-1.91 (m, 3H), 1.75-1.67 (m, 2H), 1.53-1.07 (m, 24H). HRMS (ESI-TOF) m/z calcd for C₆₆H₇₈N₇O₈ [M+H]⁺ 1096.5912, found 1096.5931.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenyl-cyclopropyl)carbamoyl)pyrrolidine-3-carboxamido)-dodecyl 4-(tert-butoxy)benzoate (Compound 76)

A reaction solution of Compound 7 (19 mg, 0.020 mmol), 4-tert-butoxybenzoic acid (3.9 mg, 0.020 mmol), DMAP (2.4 mg, 0.020 mmol), and EDCI·HCl (5.7 mg, 0.030 mmol) in anhydrous DMF (0.2 mL) and CH₂Cl₂ (0.2 mL) was stirred at 40° C. (or rt) for 8 h (or overnight, ca. 18 h). The reaction was quenched by the addition of aqueous 1 N HCl (0.2 mL) and the solution was diluted with H₂O (2 mL). The mixture was extracted with EtOAc (3.0 mL×3), and the combined organic phase was dried over anhydrous Na₂SO₄, filtered and concentrated under reduced pressure. PTLC (SiO₂, 10% MeOH—CH₂Cl₂) afforded Compound 76 (13 mg, 56%) as a white solid. ¹H NMR (CDCl₃, 500 MHz) δ 7.95 (dd, J=8.8, 2.1 Hz, 2H), 7.55-7.51 (m, 4H), 7.27-7.07 (m, 15H), 7.00 (dd, J=8.7, 1.4 Hz, 2H), 4.28-4.25 (m, 2H), 3.80-3.60 (m, 8H), 3.33-3.09 (m, 4H), 3.04-2.93 (m, 2H), 2.90-2.81 (m, 3H), 2.05-1.95 (m, 3H), 1.75-1.69 (m, 2H), 1.48-1.38 (m, 11H), 1.33-1.06 (m, 22H). HRMS (ESI-TOF) m/z calcd for C₇₀H₈₅N₆O₉ [M+H]⁺ 1153.6378, found 1153.6407.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenyl-cyclopropyl)carbamoyl)pyrrolidine-3-carboxamido)-dodecyl 4-hydroxybenzoate (Compound 75)

A solution of Compound 76 (23 mg, 0.020 mmol) in TFA (0.20 mL) and anhydrous CH₂Cl₂ (0.20 mL) was stirred at rt for 8 h. The liquids were concentrated under reduced pressure. PTLC (SiO₂, 10% MeOH—CH₂Cl₂) afforded Compound 75 (15 mg, 68%) as a white solid. ¹H NMR (CDCl₃, 500 MHz) δ 7.85 (dd, J=8.7, 2.5 Hz, 2H), 7.54-7.49 (m, 4H), 7.27-7.03 (m, 15H), 6.83 (d, J=8.7 Hz, 2H), 4.29-4.25 (m, 2H), 3.82-3.56 (m, 8H), 3.35-2.95 (m, 6H), 2.90-2.81 (m, 3H), 2.05-1.93 (m, 3H), 1.73-1.68 (m, 2H), 1.45-1.05 (m, 24H). HRMS (ESI-TOF) m/z calcd for C₆₆H₇₇N₆O₉ [M+H]⁺ 1097.5752, found 1097.5784.

### 12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenyl-cyclopropyl)carbamoyl)pyrrolidine-3-carboxamido)-dodecyl tert-butyl terephthalate (Compound 77)

A reaction solution of Compound 7 (9.8 mg, 0.010 mmol), 4-(tert-butoxycarbonyl)benzoic acid (3.3 mg, 0.015 mmol), DMAP (1.2 mg, 0.010 mmol), and EDCI·HCl (2.9 mg, 0.015 mmol) in anhydrous DMF (0.1 mL) and CH₂Cl₂ (0.1 mL) was stirred at 40° C. (or rt) for 8 h (or overnight, ca. 18 h). The reaction was quenched by the addition of aqueous 1 N HCl (0.1 mL) and the solution was diluted with H₂O (1 mL). The mixture was extracted with EtOAc (1.5 mL×3), and the combined organic phase was dried over anhydrous Na₂SO₄, filtered and concentrated under reduced pressure. PTLC (SiO₂, 10% MeOH—CH₂Cl₂) afforded Compound 77 (8.1 mg, 69%) as a white solid. ¹H NMR (CDCl₃, 400 MHz) δ 8.09-8.02 (m, 4H), 7.56-7.51 (m, 4H), 7.34 (br, 1H), 7.28-7.08 (m, 15H), 6.73 (br, 1H), 6.64 (br, 1H), 4.33-4.30 (m, 2H), 3.85-3.61 (m, 8H), 3.33-3.07 (m, 4H), 3.05-2.80 (m, 5H), 2.05-1.95 (m, 3H), 1.80-1.72 (m, 2H), 1.60 (s, 9H), 1.49-1.04 (m, 24H). HRMS (ESI-TOF) m/z calcd for C₇₁H₈₅N₆O₁₀ [M+H]⁺ 1181.6327, found 1181.6324.

### 4-(((12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)-dodecyl)oxy)carbonyl)benzoic acid (Compound 78)

A reaction solution of Compound 77 (8.0 mg, 0.0068 mmol) in TFA (0.2 mL) and CH₂Cl₂ (0.2 mL) was stirred at rt for 8 h. The solvents were removed under reduced pressure. PTLC (SiO₂, 15% MeOH—CH₂Cl₂) afforded Compound 78 (3.4 mg, 44%) as a white solid. ¹H NMR (CD₃OD, 400 MHz) δ 8.11-8.07 (m, 4H), 7.62 (s, 4H), 7.27-7.05 (m, 15H), 4.33 (td, J=6.5, 1.2 Hz, 2H), 4.03-3.97 (m, 2H), 3.79-3.60 (m, 6H), 3.36-3.01 (m, 6H), 2.91-2.77 (m, 3H), 2.09-1.91 (m, 3H), 1.81-1.73 (m, 2H), 1.54-1.07 (m, 24H). HRMS (ESI-TOF) m/z calcd for C₆₇H₇₇N₆O₁₀ [M+H]⁺ 1125.5701, found 1125.5719.

### tert-Butyl 4-((2-((12-((3S,4S)-1-(4-((3S,4S)-3,4-bis-(((1S,2R)-2-phenylcyclopropyl)carbamoyl)-pyrrolidine-1-carbonyl)benzoyl)-4-(((1S,2R)-2-phenylcyclopropyl)-carbamoyl)pyrrolidine-3-carboxamido)dodecyl)oxy)-2-oxoethyl)carbamoyl)benzoate (Compound 79)

Step 1: A solution of Compound 35 (57 mg, 0.050 mmol) in 4 N HCl/1,4-dioxane (2.0 mL) was stirred at rt for 2 h. The solvent was removed and the residue was further dried under high vacuum to afford the amine Compound 43 hydrochloride salt. HRMS (ESI-TOF) m/z calcd for C₆₁H₇₆N₇O₈ [M−Cl]⁺ 1034.5755, found 1034.5759.

Step 2: A solution of the amine hydrochloride salt (0.010 mmol), 4-(tert-butoxycarbonyl)benzoic acid (11 mg, 0.050 mmol), DMAP (3.0 mg, 0.025 mmol), Et₃N (10 μL, 0.075 mmol), and EDCI·HCl (14 mg, 0.075 mmol) in anhydrous DMF (0.25 mL) and CH₂Cl₂ (0.25 mL) was stirred at 40° C. overnight (ca. 18 h). The reaction was quenched by the addition of H₂O (1 mL), and the mixture was extracted with EtOAc (1.5 mL×3). The combined organic phase was dried over anhydrous Na₂SO₄, filtered, and concentrated under reduced pressure. PTLC (SiO₂, 10% MeOH—CH₂Cl₂) afforded Compound 79 (40 mg, 65%) as a white solid. ¹H NMR (CDCl₃, 400 MHz) δ 8.03 (dd, J=8.4, 1.9 Hz, 2H), 7.84 (d, J=8.2 Hz, 2H), 7.55-7.50 (m, 4H), 7.34 (br, 1H), 7.27-7.07 (m, 15H), 6.95 (br, 1H), 6.90 (br, 1H), 6.78 (br, 1H), 6.69 (br, 1H), 4.22-4.16 (m, 4H), 3.84-3.60 (m, 8H), 3.33-3.08 (m, 4H), 3.05-2.80 (m, 5H), 2.04-1.95 (m, 3H), 1.69-1.57 (m, 11H), 1.49-1.38 (m, 2H), 1.35-1.06 (m, 22H). HRMS (ESI-TOF) m/z calcd for C₇₃H₈₈N₇O₁₁ [M+H]⁺ 1238.6542, found 1238.6539.

### 4-((2-((12-((3S,4S)-1-(4-((3S,4S)-3,4-Bis(((1S,2R)-2-phenylcyclopropyl)carbamoyl)pyrrolidine-1-carbonyl)-benzoyl)-4-(((1S,2R)-2-phenylcyclopropyl)carbamoyl)-pyrrolidine-3-carboxamido)-dodecyl)oxy)-2-oxoethyl)carbamoyl)benzoic acid (Compound 80)

A solution of Compound 79 (15 mg, 0.012 mmol) in TFA (0.15 mL) and anhydrous CH₂Cl₂ (0.15 mL) was stirred at rt overnight (ca. 18 h). The liquids were concentrated under reduced pressure. PTLC (SiO₂, 15% MeOH—CH₂Cl₂) afforded Compound 80 (9.6 mg, 68%) as a white solid. ¹H NMR (CD₃OD, 400 MHz) δ 8.10 (dd, J=8.4, 1.6 Hz, 2H), 7.92 (d, J=8.4 Hz, 2H), 7.62 (s, 4H), 7.27-7.05 (m, 15H), 4.16-4.13 (m, 2H), 4.11 (s, 2H), 4.03-3.97 (m, 2H), 3.80-3.60 (m, 6H), 3.35-3.02 (m, 6H), 2.91-2.77 (m, 3H), 2.09-1.91 (m, 3H), 1.67-1.59 (m, 2H), 1.54-1.40 (m, 2H), 1.36-1.07 (m, 22H). ¹³C NMR (CD₃OD, 150 MHz) δ 174.51, 174.48, 173.9, 173.85, 173.82, 173.1, 172.5, 171.5, 170.9, 169.8, 169.5, 142.24, 142.21, 142.15, 142.14, 142.11, 139.3, 138.8, 135.9, 131.0, 129.5, 128.6, 127.4, 127.3, 127.22, 127.20, 66.6, 53.4, 53.3, 50.6, 50.5, 50.4, 47.8, 47.7, 47.5, 42.9, 40.8, 40.7, 35.1, 33.6, 33.5, 33.2, 30.91, 30.90, 30.85, 30.83, 30.82, 30.77, 30.7, 30.61, 30.56, 30.50, 30.47, 30.46, 30.4, 29.8, 28.10, 28.06, 27.1, 26.3, 25.5, 25.43, 25.39, 25.3, 23.9, 16.31, 16.29, 16.26, 14.6. HRMS (ESI-TOF) m/z calcd for C₆₉H₈₀N₇O₁₁ [M+H]⁺ 1182.5916, found 1182.5912.

### (E)-tert-Butyl 4-((2-((12-((3S,4S)-1-(4-((3S,4S)-3,4-bis(((1S,2R)-2-phenylcyclopropyl)carbamoyl)-pyrrolidine-1-carbonyl)benzoyl)-4-(((1S,2R)-2-phenylcyclopropyl)carbamoyl)pyrrolidine-3-carboxamido)dodecyl)oxy)-2-oxoethyl)amino)-4-oxobut-2-enoate (Compound 81)

Step 1: A solution of Compound 35 (57 mg, 0.050 mmol) in 4 N HCl/1,4-dioxane (2.0 mL) was stirred at rt for 2 h. The solvent was removed and the residue was further dried under high vacuum to afford the amine Compound 43 hydrochloride salt.

Step 2: A solution of the amine hydrochloride salt (0.050 mmol), (E)-4-(tert-butoxy)-4-oxobut-2-enoic acid (8.6 mg, 0.050 mmol), DMAP (3.0 mg, 0.025 mmol), Et₃N (10 μL, 0.075 mmol), and EDCI·HCl (14 mg, 0.075 mmol) in anhydrous DMF (0.25 mL) and CH₂Cl₂ (0.25 mL) was stirred at 40° C. overnight (ca. 18 h). The reaction was quenched by the addition of H₂O (1 mL), and the mixture was extracted with EtOAc (1.5 mL×3). The combined organic phase was dried over anhydrous Na₂SO₄, filtered, and concentrated under reduced pressure. PTLC (SiO₂, 10% MeOH—CH₂Cl₂) afforded Compound 81 (33 mg, 55%) as a white solid. ¹H NMR (CDCl₃, 400 MHz) δ 7.56-7.51 (m, 4H), 7.34 (br, 1H), 7.31 (br, 1H), 7.28-7.07 (m, 15H), 6.93-6.87 (m, 2H), 6.81 (br, 1H), 6.76-6.72 (m, 2H), 4.14 (q, J=6.4 Hz, 2H), 4.08-4.04 (m, 2H), 3.82-3.60 (m, 8H), 3.35-3.11 (m, 4H), 3.07-2.81 (m, 5H), 2.06-1.95 (m, 3H), 1.64-1.58 (m, 2H), 1.49-1.40 (m, 11H), 1.32-1.05 (m, 22H). HRMS (ESI-TOF) m/z calcd for C₆₉H₈₆N₇O₁₁ [M+H]⁺ 1188.6385, found 1188.6371.

### (E)-4-((2-((12-((3S,4S)-1-(4-((3S,4S)-3,4-Bis(((1S,2R)-2-phenylcyclopropyl)carbamoyl)-pyrrolidine-1-carbonyl)benzoyl)-4-(((1S,2R)-2-phenylcyclopropyl)carbamoyl)pyrrolidine-3-carboxamido)dodecyl)oxy)-2-oxoethyl)amino)-4-oxobut-2-enoic acid (Compound 82)

A solution of Compound 81 (22 mg, 0.018 mmol) in TFA (0.18 mL) and anhydrous CH₂Cl₂ (0.18 mL) was stirred at rt overnight (ca. 18 h). The liquids were concentrated under reduced pressure. PTLC (SiO₂, 15% MeOH—CH₂Cl₂) afforded Compound 82 (8.0 mg, 39%) as a white solid. ¹H NMR (CD₃OD, 400 MHz) δ 7.62 (s, 4H), 7.27-7.06 (m, 15H), 6.91 (d, J=15.5 Hz, 1H), 6.76 (d, J=15.4 Hz, 1H), 4.14-4.10 (m, 2H), 4.03-3.96 (m, 4H), 3.79-3.60 (m, 6H), 3.36-3.02 (m, 6H), 2.91-2.78 (m, 3H), 2.09-1.91 (m, 3H), 1.66-1.59 (m, 2H), 1.54-1.41 (m, 2H), 1.33-1.08 (m, 22H). ¹³C NMR (CD₃OD, 150 MHz) δ 174.53, 174.51, 173.9, 173.8, 173.1, 172.5, 171.2, 170.9, 167.53, 142.3, 142.22, 142.17, 142.15, 142.13, 139.33, 139.32, 135.1, 134.9, 129.5, 128.6, 127.4, 127.3, 127.23, 127.20, 66.6, 53.4, 53.3, 50.54, 50.51, 50.4, 49.7, 47.9, 47.7, 47.5, 42.4, 40.8, 40.7, 35.2, 33.6, 33.5, 33.2, 30.91, 30.90, 30.85, 30.82, 30.79, 30.77, 30.7, 30.61, 30.56, 30.50, 30.46, 30.45, 30.40, 29.8, 28.11, 28.07, 27.1, 26.3, 25.5, 25.43, 25.40, 25.36, 23.9, 16.3, 16.25, 16.23, 14.6. HRMS (ESI-TOF) m/z calcd for C₆₅H₇₈N₇O₁₁ [M+H]⁺ 1132.5759, found 1132.5737.

### tert-Butyl 2-((2-((12-((3S,4S)-1-(4-((3S,4S)-3,4-bis(((1S,2R)-2-phenylcyclopropyl)carbamoyl)-pyrrolidine-1-carbonyl)benzoyl)-4-(((1S,2R)-2-phenylcyclopropyl)carbamoyl)pyrrolidine-3-carboxamido)dodecyl)oxy)-2-oxoethyl)amino)-2-oxoacetate (Compound 83)

Step 1: A solution of Compound 35 (48 mg, 0.0423 mmol) in 4 N HCl/1,4-dioxane (1.2 mL) was stirred at rt for 1 h. The solvent was removed and the residue was further dried under high vacuum to afford the amine Compound 43 hydrochloride salt.

Step 2: A solution of the amine hydrochloride salt (0.0423 mmol) in anhydrous pyridine (0.4 mL) was treated with Et₃N (5.9 μL) and tert-butyl 2-chloro-2-oxoacetate (10 mg, 0.0634 mmol) dissolved in anhydrous CH₂Cl₂ (0.4 mL). The reaction solution was stirred at 40° C. for several hours and then quenched by the addition of H₂O (1 mL), and the mixture was extracted with EtOAc (3.0 mL×3). The combined organic phase was dried over anhydrous Na₂SO₄, filtered, and concentrated under reduced pressure. PTLC (SiO₂, 10% MeOH—CH₂Cl₂) afforded Compound 83 (25 mg, 51%) as a white solid. ¹H NMR (CDCl₃, 600 MHz) d 7.58 (br, 1H), 7.55-7.51 (m, 4H), 7.44-7.30 (m, 3H), 7.25-7.06 (m, 15H), 6.86-6.79 (m, 1H), 4.16-4.13 (m, 2H), 4.07-4.06 (m, 2H), 3.82-3.60 (m, 8H), 3.32-3.09 (m, 4H), 3.04-2.94 (m, 2H), 2.89-2.81 (m, 3H), 2.04-1.95 (m, 3H), 1.64-1.61 (m, 2H), 1.54 (s, 9H), 1.48-1.41 (m, 2H), 1.32-1.06 (m, 22H). HRMS (ESI-TOF) m/z calcd for C₆₇H₈₄N₇O₁₁ [M+H]⁺ 1162.6229, found 1162.6265.

### 2-((2-((12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)carbamoyl)pyrrolidine-3-carboxamido)-dodecyl)oxy)-2-oxoethyl)amino)-2-oxoacetic acid (Compound 84)

A solution of Compound 83 (20 mg, 0.017 mmol) in TFA (0.17 mL) and anhydrous CH₂Cl₂ (0.17 mL) was stirred at rt overnight (ca. 18 h). The liquids were concentrated under reduced pressure. PTLC (SiO₂, 15% MeOH—CH₂Cl₂) afforded Compound 84 (12 mg, 64%) as a white solid. ¹H NMR (CD₃OD, 600 MHz) d 7.62 (s, 4H), 7.26-7.20 (m, 6H), 7.16-7.11 (m, 6H), 7.09-7.06 (m, 3H), 4.12-4.10 (m, 2H), 4.01-3.97 (m, 4H), 3.79-3.61 (m, 6H), 3.35-3.03 (m, 6H), 2.90-2.78 (m, 3H), 2.08-1.93 (m, 3H), 1.64-1.59 (m, 2H), 1.53-1.43 (m, 2H), 1.33-1.10 (m, 22H). ¹³C NMR (CD₃OD, 150 MHz) δ 174.5, 173.9, 173.3, 172.7, 171.3, 170.9, 142.3, 142.2, 142.1, 139.3, 129.5, 128.6, 127.4, 127.3, 127.2, 66.5, 53.3, 50.5, 50.4, 47.8, 47.7, 47.5, 42.4, 40.8, 40.7, 33.6, 33.5, 30.9, 30.8, 30.7, 30.6, 30.5, 30.4, 29.8, 28.1, 27.0, 25.5, 25.4, 25.3, 16.3, 16.2. HRMS (ESI-TOF) m/z calcd for C₆₃H₇₆N₇O₁₁ [M+H]⁺ 1106.5603, found 1106.5630.

### tert-Butyl 4-((2-((12-((3S,4S)-1-(4-((3S,4S)-3,4-bis-(((1S,2R)-2-phenylcyclopropyl)carbamoyl)-pyrrolidine-1-carbonyl)benzoyl)-4-(((1S,2R)-2-phenylcyclopropyl)-carbamoyl)pyrrolidine-3-carboxamido)dodecyl)-oxy)ethyl)carbamoyl)benzoate (Compound 85)

Step 1: A solution of Compound 48 (42 mg, 0.037 mmol) in 4 N HCl/1,4-dioxane (0.8 mL) was stirred at rt for 1 h. The solvent was removed and the residue was further dried under high vacuum to afford the amine hydrochloride salt.

Step 2: A solution of the amine hydrochloride salt (0.037 mmol), 4-(tert-butoxycarbonyl)benzoic acid (8.2 mg, 0.037 mmol), DMAP (2.2 mg, 0.018 mmol), Et₃N (8.0 μL, 0.055 mmol), and EDCI·HCl (11 mg, 0.055 mmol) in anhydrous DMF (0.20 mL) and CH₂Cl₂ (0.20 mL) was stirred at 40° C. overnight (ca. 18 h). The reaction was quenched by the addition of H₂O (1 mL), and the mixture was extracted with EtOAc (3.0 mL×3). The combined organic phase was dried over anhydrous Na₂SO₄, filtered, and concentrated under reduced pressure. PTLC (SiO₂, 10% MeOH—CH₂Cl₂) afforded Compound 85 (32 mg, 71%) as a white solid. ¹H NMR (CDCl₃, 600 MHz) d 8.02 (dd, J=8.2, 3.8 Hz, 2H), 7.79 (d, J=8.0 Hz, 2H), 7.53-7.50 (m, 4H), 7.44-7.39 (m, 2H), 7.25-7.05 (m, 15H), 6.92-6.85 (m, 1H), 6.73-6.69 (m, 1H), 3.80-3.57 (m, 12H), 3.46-3.43 (m, 2H), 3.33-3.24 (m, 2H), 3.21-3.08 (m, 2H), 3.04-2.96 (m, 2H), 2.89-2.81 (m, 3H), 2.04-1.93 (m, 3H), 1.59-1.53 (m, 11H), 1.48-1.38 (m, 2H), 1.33-1.05 (m, 22H). ¹³C NMR (CDCl₃, 150 MHz) δ 171.9, 171.8, 171.3, 170.5, 170.0, 168.6, 168.5, 168.4, 166.9, 165.1, 140.4, 138.1, 138.0, 137.8, 134.7, 129.8, 128.6, 127.6, 127.5, 127.0, 126.5, 126.4, 126.3, 81.8, 71.5, 69.1, 52.1, 49.0, 48.9, 47.8, 47.7, 47.6, 45.7, 45.6, 45.5, 45.3, 40.1, 40.0, 32.4, 32.3, 29.9, 29.7, 29.6, 29.5, 29.4, 28.3, 27.1, 26.3, 24.9, 24.8, 24.7, 16.1, 15.8. HRMS (ESI-TOF) m/z calcd for C₇₃H₉₀N₇O₁₀ [M+H]⁺ 1224.6749, found 1224.6774.

### 4-((2-((12-((3S,4S)-1-(4-((3S,4S)-3,4-Bis(((1S,2R)-2-phenylcyclopropyl)carbamoyl)pyrrolidine-1-carbonyl)benzoyl)-4-(((1S,2R)-2-phenylcyclopropyl)-carbamoyl)pyrrolidine-3-carboxamido)dodecyl)-oxy)ethyl)carbamoyl)benzoic acid (Compound 86)

A solution of Compound 85 (67 mg, 0.055 mmol) in TFA (0.55 mL) and anhydrous CH₂Cl₂ (0.55 mL) was stirred at rt overnight (ca. 18 h). The liquids were concentrated under reduced pressure. PTLC (SiO₂, 15% MeOH—CH₂Cl₂) afforded Compound 86 (40 mg, 62%) as a white solid. ¹H NMR (CD₃OD, 500 MHz) d 8.07 (dd, J=8.4, 2.6 Hz, 2H), 7.88 (d, J=8.0 Hz, 2H), 7.61 (s, 4H), 7.26-7.04 (m, 15H), 4.02-3.97 (m, 2H), 3.78-3.68 (m, 4H), 3.65-3.55 (m, 6H), 3.47 (t, J=6.6 Hz, 2H), 3.35-3.02 (m, 6H), 2.90-2.77 (m, 3H), 2.08-1.91 (m, 3H), 1.56-1.41 (m, 4H), 1.35-1.07 (m, 22H). ¹³C NMR (CD₃OD, 150 MHz) δ 174.5, 173.8, 173.1, 172.5, 170.9, 169.9, 169.6, 142.25, 142.22, 142.15, 142.12, 139.4, 139.3, 136.1, 130.9, 129.5, 128.6, 128.5, 127.4, 127.3, 127.2, 72.2, 70.1, 53.4, 53.3, 50.5, 50.4, 47.8, 47.7, 47.5, 41.2, 40.8, 40.7, 33.6, 33.5, 30.91, 30.87, 30.8, 30.7, 30.63, 30.57, 30.5, 28.11, 28.07, 27.4, 25.5, 25.43, 25.39, 25.3, 16.30, 16.26. HRMS (ESI-TOF) m/z calcd for C₆₉H₈₂N₇O₁₀ [M+H]⁺ 1168.6123, found 1168.6138.

### Syntheses of R Group Precursors

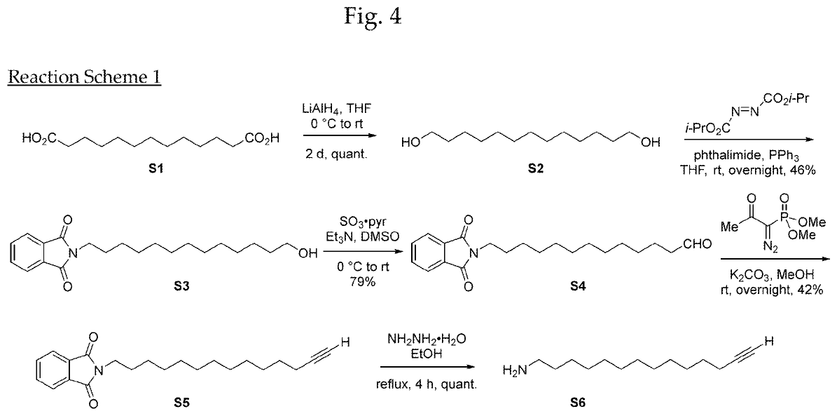

Syntheses for several R group precursor reactants are illustrated in the Reaction Schemes shown in FIG. 4 through FIG. 10, and the detailed synthetic procedures set out below.

FIG. 4 illustrates Reaction Scheme 1 that shows the reactions leading to Compound S6, whose detailed synthesis is set out below.

Tridecane-1,13-diol (Compound S2)³⁰

A solution of 1,11-undecanedicarboxylic acid (Compound S1, 4.9 g, 20 mmol) in anhydrous THF (40 mL) was added dropwise by an addition funnel to the slurry mixture of LiAlH₄ (1.9 g, 50 mmol) in anhydrous THF (40 mL) at 0° C. The resulting reaction mixture was stirred at rt for 2 days. The mixture was cooled to 0° C. and carefully quenched by the addition of H₂O (80 mL) and aqueous 1 N HCl (20 mL). The mixture was extracted with EtOAc (120 mL×3), and the combined organic phase was washed with saturated aqueous NaCl (300 mL), dried over anhydrous Na₂SO₄, filtered and concentrated in vacuo to afford Compound S2 (4.3 g, 99%) as a white solid, which was used without further purification. ¹H NMR (DMSO-d₆, 500 MHz) δ 4.31 (s, 2H), 3.38-3.35 (m, 4H), 1.39 (p, J=6.7 Hz, 4H), 1.27-1.22 (m, 18H).

### 2-(13-Hydroxytridecyl)isoindoline-1,3-dione (Compound S3)³¹

A solution of phthalimide (1.8 g, 12 mmol), Compound S2 (3.9 g, 18 mmol), and Ph₃P (3.1 g, 12 mmol) in anhydrous THF (30 mL) was treated with diisopropyl azodicarboxylate (2.4 mL, 12 mmol) dropwise at 0° C. The reaction solution was warmed to rt and stirred overnight (ca. 18 h). The solvent was removed under reduced pressure. Flash chromatography (SiO₂, 2-8% EtOAc—CH₂Cl₂ gradient) afforded Compound S3 (1.9 g, 46%) as a white solid. ¹H NMR (CDCl₃, 400 MHz) δ 7.84 (dd, J=5.5, 3.1 Hz, 2H), 7.70 (dd, J=5.4, 3.0 Hz, 2H), 3.69-3.62 (m, 4H), 1.67 (p, J=7.5 Hz, 2H), 1.60-1.53 (m, 2H), 1.36-1.25 (m, 18H). HRMS (ESI-TOF) m/z calcd for C₂₁H₃₂NO₃ [M+H]⁺ 346.2377, found 346.2387.

### 13-(1,3-Dioxoisoindolin-2-yl)tridecanal (Compound S4)

A solution of Compound S3 (0.34 g, 1.0 mmol) in DMSO (2.5 mL) was treated with Et₃N (1.4 mL, 10 mmol) and a solution of pyridine sulfur trioxide (0.56 g, 3.5 mmol) in DMSO (3.0 mL) at 0° C. The reaction solution was stirred at rt until full consumption of Compound S3, and then quenched by the addition of H₂O (10 mL). The mixture was extracted with Et₂O (15 mL×3), and the combined organic phase was dried over anhydrous Na₂SO₄, filtered and concentrated under reduced pressure. Flash chromatography (SiO₂, 5-10% EtOAc-hexanes gradient) afforded Compound S4 (0.27 g, 79%) as a white solid. ¹H NMR (CDCl₃, 400 MHz) δ 9.76 (s, 1H), 7.84 (dd, J=5.4, 3.0 Hz, 2H), 7.71 (dd, J=5.5, 3.0 Hz, 2H), 3.67 (t, J=7.3 Hz, 2H), 2.41 (td, J=7.4, 1.9 Hz, 2H), 1.70-1.58 (m, 4H), 1.35-1.25 (m, 16H). HRMS (ESI-TOF) m/z calcd for C₂₁H₃₀NO₃ [M+H]⁺ 344.2226, found 344.2229.

### 2-(Tetradec-13-yn-1-yl) isoindoline-1,3-dione (Compound S5)

A solution of Compound S4 (166 mg, 0.480 mmol) and K₂CO₃(133 mg, 0.960 mmol) in anhydrous MeOH (5.0 mL) was treated with dimethyl-(1-diazo-2-oxopropyl)phosphonate (87.0 μL, 0.580 mmol) at rt. The reaction mixture was stirred at rt overnight (ca. 18 h) before being quenched by the addition of H₂O (10 mL) and neutralized with 10% citric acid. The mixture was extracted with EtOAc (30 mL×3), and the combined organic phase was dried over anhydrous Na₂SO₄, filtered and concentrated under reduced pressure. Flash chromatography (SiO₂, 5-10% EtOAc-hexanes gradient) afforded Compound S5 (68 mg, 42%) as a white solid. ¹H NMR (CDCl₃, 400 MHz) δ 7.84 (dd, J=5.4, 3.0 Hz, 2H), 7.70 (dd, J=5.4, 3.0 Hz, 2H), 3.69-3.65 (m, 2H), 2.17 (td, J=7.1, 2.7 Hz, 2H), 1.93 (t, J=2.6 Hz, 1H), 1.67 (p, J=7.4 Hz, 2H), 1.54-1.48 (m, 2H), 1.35-1.25 (m, 16H). HRMS (ESI-TOF) m/z calcd for C₂₂H₃₀NO₂ [M+H]⁺ 340.2277, found 340.2272.

### Tetradec-13-yn-1-amine (Compound S6)

A solution of S5 (26 mg, 0.076 mmol) in hydrazine hydrate (0.30 mL) and EtOH (0.60 mL) was stirred at reflux for 1 h. The reaction solution was cooled to rt and adjusted to a weakly basic solution with the addition of aqueous 6 N HCl. The mixture was further diluted with water (1 mL) and extracted with CH₂Cl₂ (2 mL×4), and the combined organic phase was dried over anhydrous Na₂SO₄, filtered, and concentrated in vacuo to afford Compound S6 (15 mg, 94%) as a white solid, which was used without further purification. ¹H NMR (CDCl₃, 500 MHz) δ 2.81 (t, J=7.5 Hz, 2H), 2.17 (td, J=7.1, 2.7 Hz, 2H), 1.93 (t, J=2.6 Hz, 2H), 1.59 (p, J=7.4 Hz, 2H), 1.51 (p, J=7.2 Hz, 2H), 1.39-1.24 (m, 16H). HRMS (ESI-TOF) m/z calcd for C₁₄H₂₈N [M+H]⁺ 210.2222, found 210.2225.

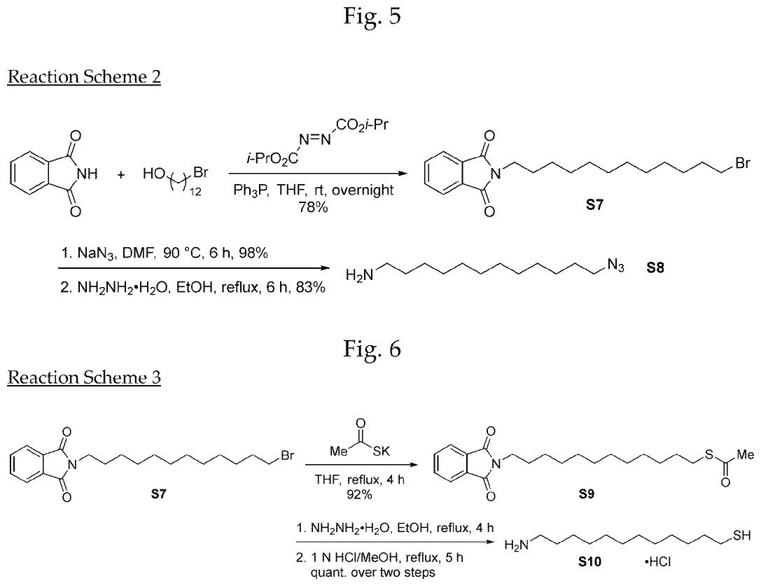

FIG. 5 illustrates Reaction Scheme 2 that shows the reactions leading to Compound S8, whose detailed synthesis is set out below.

### 2-(12-Bromododecyl)isoindoline-1,3-dione (Compound S7)³²

A solution of phthalimide (0.74 g, 5.0 mmol), 12-bromo-1-dodecanol (1.9 g, 7.5 mmol), and Ph₃P (1.3 g, 5.0 mmol) in anhydrous THF (12.5 mL) was treated with diisopropyl azodicarboxylate (0.98 mL, 5.0 mmol) dropwise at 0° C. The reaction solution was warmed to rt and stirred overnight (ca. 18 h). The solvent was removed under reduced pressure. Flash chromatography (SiO₂, 2-10% EtOAc-hexanes gradient) afforded Compound S7 (1.53 g, 78%) as a white solid. ¹H NMR (CDCl₃, 400 MHz) δ 7.84 (dd, J=5.4, 3.1 Hz, 2H), 7.70 (dd, J=5.4, 3.0 Hz, 2H), 3.69-3.65 (m, 2H), 3.40 (t, J=6.9 Hz, 2H), 1.85 (p, J=7.0 Hz, 2H), 1.67 (p, J=7.3 Hz, 2H), 1.45-1.37 (m, 2H), 1.34-1.25 (m, 14H). HRMS (ESI-TOF) m/z calcd for C₂₀H₂₉BrNO₂ [M+H]⁺ 394.1382, found 394.1389.

### 12-Azidododecan-1-amine (Compound S8)³³

Step 1: A solution of Compound S7 (0.39 g, 1.0 mmol) in anhydrous DMF (5.0 mL) was treated with NaN₃ (0.32 g, 5.0 mmol) in one portion at rt. The reaction mixture was stirred at 90° C. for 6 h, and cooled to rt. The reaction mixture was quenched by the addition of H₂O (10 mL) and extracted with EtOAc (15 mL×3). The combined organic phase was dried over anhydrous Na₂SO₄, filtered and concentrated under reduced pressure. Flash chromatography (SiO₂, 8-12% EtOAc-hexanes gradient) afforded the azide Compound S8 (0.35 g, 98%) as a colorless oil.

Step 2: A solution of Compound S8 (0.35 g, 0.98 mmol) in hydrazine hydrate (2.5 mL) and EtOH (5.0 mL) was stirred at reflux for 6 h, and cooled to rt. The reaction solution was quenched by the dropwise addition of aqueous 6 N HCl until the formation of white solid. The precipitated white solid was collected by filtration and washed with H₂O. The filtrate was washed with CH₂Cl₂ (15 mL×3) before the aqueous layer was basified with the addition of aqueous 1 N NaOH and extracted with CH₂Cl₂ (45 mL×3). The combined organic phase was dried over anhydrous Na₂SO₄, filtered, and concentrated in vacuo to afford Compound S8 (185 mg, 83%) as a yellow oil, which was used without further purification. ¹H NMR (CDCl₃, 400 MHz) δ 3.25 (t, J=6.9 Hz, 2H), 2.67 (t, J=7.0 Hz, 2H), 1.59 (p, J=7.0 Hz, 2H), 1.45-1.27 (m, 18H). HRMS (ESI-TOF) m/z calcd for C₁₂H₂₇N₄[M+H]⁺ 227.2236, found 227.2234.

FIG. 6 illustrates Reaction Scheme 3 that shows the reactions leading to Compound S10, whose detailed synthesis is set out below.

### S-(12-(1,3-Dioxoisoindolin-2-yl)dodecyl) ethanethioate (Compound S9)³⁴

A reaction mixture of Compound S7 (0.59 g, 1.5 mmol) and potassium thioacetate (0.51 g, 4.5 mmol) in anhydrous THF (15 mL) was stirred at reflux for 4 h, then cooled to rt. The reaction mixture was quenched with the addition of H₂O (15 mL) and extracted with EtOAc (15 mL×3). The combined organic phase was dried over anhydrous Na₂SO₄, filtered and concentrated under reduced pressure. Flash chromatography (SiO₂, 2-10% EtOAc-hexanes gradient) afforded Compound S9 (0.54 g, 92%) as a pale-yellow solid. ¹H NMR (CDCl₃, 400 MHz) δ 7.84 (dd, J=5.4, 3.1 Hz, 2H), 7.70 (dd, J=5.5, 3.0 Hz, 2H), 3.69-3.65 (m, 2H), 2.85 (t, J=7.3 Hz, 2H), 2.31 (s, 3H), 1.66 (p, J=7.6 Hz, 2H), 1.59-1.51 (m, 2H), 1.35-1.24 (m, 16H). HRMS (ESI-TOF) m/z calcd for C₂₂H₃₂NO₃S [M+H]⁺ 390.2097, found 390.2108.

### 12-Aminododecane-1-thiol hydrochloride (Compound S10)³⁴

A solution of Compound S9 (0.15 g, 0.38 mmol) in hydrazine hydrate (1.3 mL) and EtOH (2.6 mL) was stirred at reflux for 4 h, then cooled to rt. The solvents were removed under reduced pressure. The remaining residue was treated with 1.25 N HCl/MeOH (4.0 mL) and the resulting solution was stirred at reflux for 5 h. The reaction mixture was cooled to rt before being diluted with H₂O (3 mL) and aqueous 6 N HCl (1 mL). The mixture was extracted with CH₂Cl₂ (6 mL×4). The combined organic phase was dried over anhydrous Na₂SO₄, filtered and concentrated under reduced pressure to afford Compound S10 (96 mg, 99%) as a pale-yellow solid, which was used without further purification. ¹H NMR (DMSO-d₆, 400 MHz) δ 7.91 (br, 3H), 2.73 (t, J=7.6 Hz, 2H), 2.48-2.43 (m, 2H), 2.22 (t, J=7.7 Hz, 1H), 1.55-1.48 (m, 4H), 1.34-1.25 (m, 16H). HRMS (ESI-TOF) m/z calcd for C₁₂H₂₈NS [M−HCl+H]⁺ 218.1942, found 218.1944.

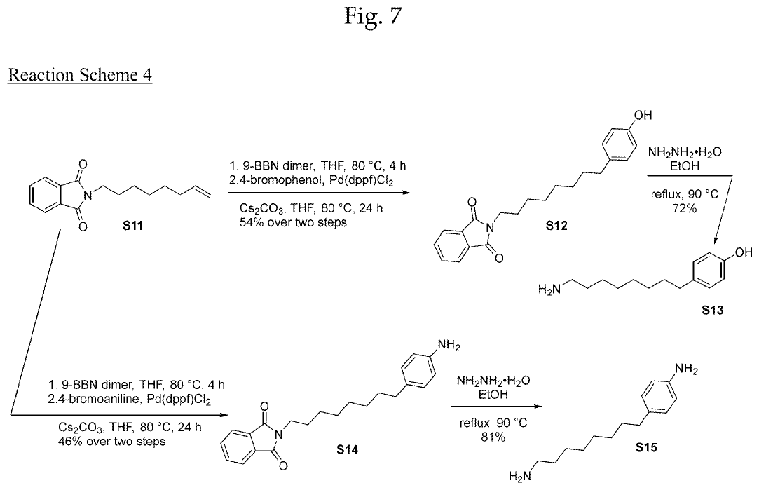

FIG. 7 illustrates Reaction Scheme 4 that shows the reactions leading to Compounds S13 and S15, whose detailed syntheses are set out below.

### 2-(Oct-7-en-1-yl)isoindoline-1,3-dione (Compound S11)³⁵

A solution of potassium phthalimide (1.0 g, 5.5 mmol) in anhydrous DMF (10 mL) was treated with 8-bromo-1-octene (0.84 mL, 5.0 mmol) dropwise at rt. The reaction solution was warmed to 60° C., stirred for 24 h, and cooled to rt. The reaction solution was quenched with the addition of H₂O (15 mL), and the mixture was extracted with Et₂O (25 mL×3). The combined organic phase was dried over anhydrous Na₂SO₄, filtered and concentrated under reduced pressure. Flash chromatography (SiO₂, 2-6% EtOAc-hexanes gradient) afforded Compound S11 (1.1 g, 85%) as a white solid. ¹H NMR (CDCl₃, 400 MHz) δ 7.84 (dd, J=5.4, 3.0 Hz, 2H), 7.70 (dd, J=5.5, 3.1 Hz, 2H), 5.79 (ddt, J=16.9, 10.2, 6.7 Hz, 1H), 4.98 (dq, J=17.2, 1.7 Hz, 1H), 4.92 (ddt, J=10.2, 2.3, 1.3 Hz, 1H), 3.69-3.66 (m, 2H), 2.06-2.00 (m, 2H), 1.67 (p, J=7.2 Hz, 2H), 1.41-1.32 (m, 6H). HRMS (ESI-TOF) m/z calcd for C₁₆H₂₀NO₂ [M+H]⁺ 258.1494, found 258.1500.

### 2-(8-(4-Hydroxyphenyl)octyl)isoindoline-1,3-dione (Compound S12)³⁶

A reaction solution of Compound S11 (0.44 g, 1.7 mmol) and 9-BBN dimer (0.29 g, 1.2 mmol) in anhydrous THF (4.5 mL) was stirred at 80° C. for 4 h, and cooled to rt. This solution was transferred to another sealed argon-filled tube charged with 4-bromophenol (0.15 g, 0.85 mmol), Pd(dppf)Cl₂ (12 mg, 0.017 mmol), and Cs₂CO₃ (0.81 g, 2.5 mmol). The resulting reaction mixture was warmed to 80° C., stirred for 24 h, and cooled to rt. The reaction mixture was quenched with the addition of H₂O (15 mL) and acidified to pH 6-7 by the addition of aqueous 1 N HCl. The mixture was extracted with EtOAc (20 mL×3), and the combined organic phase was dried over anhydrous Na₂SO₄, filtered and concentrated under reduced pressure. Flash chromatography (SiO₂, 2-5% EtOAc—CH₂Cl₂ gradient) afforded Compound S12 (0.16 g, 54%) as a white solid. ¹H NMR (CDCl₃, 400 MHz) δ 7.84 (dd, J=5.4, 3.0 Hz, 2H), 7.70 (dd, J=5.4, 3.1 Hz, 2H), 7.03 (d, J=8.4 Hz, 2H), 6.74 (d, J=8.4 Hz, 2H), 4.71 (s, 1H), 3.67 (t, J=7.3 Hz, 2H), 2.51 (t, J=7.7 Hz, 2H), 1.70-1.61 (m, 2H), 1.57-1.51 (m, 2H), 1.31-1.29 (m, 8H). HRMS (ESI-TOF) m/z calcd for C₂₂H₂₆NO₃ [M+H]⁺ 352.1907, found 352.1905.

### 4-(8-Aminooctyl)phenol (Compound S13)

A solution of Compound S12 (0.16 g, 0.45 mmol) in hydrazine hydrate (1.5 mL) and EtOH (3.0 mL) was stirred at reflux for 5 h, and cooled to rt. The reaction solution was quenched by the dropwise addition of aqueous 6 N HCl, and the white solid precipitate was removed by filtration and washed with H₂O. The filtrate was washed with CH₂Cl₂ (15 mL×3), and the aqueous layer was basified with aqueous 1 N NaOH and extracted with CH₂Cl₂ (45 mL×3). The combined organic phase was dried over anhydrous Na₂SO₄, filtered, and concentrated in vacuo to afford Compound S13 (72 mg, 72%) as a pale-yellow solid, which was used without further purification. ¹H NMR (DMSO-d₆, 400 MHz) δ 6.95 (d, J=8.4 Hz, 2H), 6.64 (d, J=8.4 Hz, 2H), 2.50-2.47 (m, 2H, buried in DMSO peak), 2.43 (t, J=7.6 Hz, 2H), 1.52-1.45 (m, 2H), 1.33-1.23 (m, 10H). HRMS (ESI-TOF) m/z calcd for C₁₄H₂₄NO [M+H]⁺ 222.1852, found 222.1858.

### 2-(8-(4-Aminophenyl)octyl)isoindoline-1,3-dione (Compound S14)³⁶

A reaction solution of Compound S11 (0.42 g, 1.6 mmol) and 9-BBN dimer (0.27 g, 1.1 mmol) in anhydrous THF (4.0 mL) was stirred at 80° C. for 4 h, and cooled to rt. This solution was transferred to another sealed argon-filled tube charged with 4-bromoaniline (0.14 g, 0.80 mmol), Pd(dppf)Cl₂ (12 mg, 0.016 mmol), and Cs₂CO₃ (0.78 g, 2.4 mmol). The resulting reaction mixture was warmed to 80° C., stirred for 24 h, and cooled to rt. The reaction mixture was quenched with the addition of H₂O (15 mL) and acidified to pH 6-7 by the addition of aqueous 1 N HCl. The mixture was extracted with EtOAc (20 mL×3), and the combined organic phase was dried over anhydrous Na₂SO₄, filtered and concentrated under reduced pressure. Flash chromatography (SiO₂, 2-10% EtOAc—CH₂Cl₂ gradient) afforded Compound S14 (0.13 g, 46%) as a white solid. ¹H NMR (CDCl₃, 400 MHz) δ 7.84 (dd, J=5.4, 3.1 Hz, 2H), 7.70 (dd, J=5.5, 3.0 Hz, 2H), 6.95 (d, J=8.0 Hz, 2H), 6.62 (d, J=8.2 Hz, 2H), 3.67 (t, J=7.3 Hz, 2H), 2.47 (t, J=7.7 Hz, 2H), 1.69-1.62 (m, 2H), 1.56-1.49 (m, 2H), 1.34-1.25 (m, 8H). HRMS (ESI-TOF) m/z calcd for C₂₂H₂₇N₂O₂ [M+H]⁺ 351.2067, found 351.2077.

### 4-(8-Aminooctyl)aniline (Compound S15)

A solution of Compound S14 (0.13 g, 0.37 mmol) in hydrazine hydrate (1.3 mL) and EtOH (2.6 mL) was stirred at reflux for 4 h, and cooled to rt. The reaction solution was quenched by the dropwise addition of aqueous 6 N HCl, and the white solid precipitate was removed by filtration and washed with H₂O. The filtrate was washed with CH₂Cl₂ (15 mL×3), and the aqueous layer was basified with the addition of aqueous 1 N NaOH and extracted with CH₂Cl₂ (45 mL×3). The combined organic phase was dried over anhydrous Na₂SO₄, filtered, and concentrated in vacuo to afford Compound S15 (66 mg, 81%) as an orange yellow solid, which was used without further purification. ¹H NMR (DMSO-d₆, 400 MHz) δ 6.81 (d, J=8.1 Hz, 2H), 6.46 (d, J=8.0 Hz, 2H), 4.77 (s, 2H), 2.51-2.49 (m, 2H, buried in DMSO peak), 2.37 (t, J=7.6 Hz, 2H), 1.50-1.42 (m, 2H), 1.35-1.28 (m, 2H), 1.26-1.21 (m, 8H). HRMS (ESI-TOF) m/z calcd for C₁₄H₂₅N₂[M+H]⁺ 221.2012, found 221.2019.

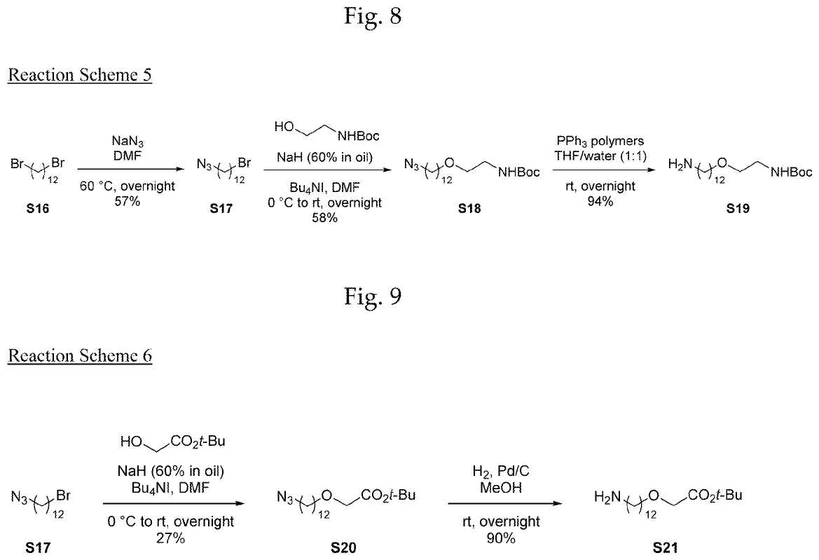

FIG. 8 illustrates Reaction Scheme 5 that shows the reactions leading to Compound S19, whose detailed syntheses are set out below.

### 1-Azido-12-bromododecane (Compound S17)³³

A reaction solution of 1,12-dibromododecane (Compound S16, 12 g, 36 mmol) in anhydrous DMF (60 mL) was treated with NaN₃ (1.6 g, 24 mmol) as a solid in one portion at rt. The reaction mixture was warmed to 60° C., stirred for 24 h, and then cooled to rt. The reaction was quenched with the addition of H₂O (60 mL), and the mixture was extracted with Et₂O (150 mL×3). The combined organic phase was dried over anhydrous Na₂SO₄, filtered and concentrated under reduced pressure. Flash chromatography (SiO₂, 0-2% ether-hexanes gradient) afforded Compound S17 (4.0 g, 57%) as a colorless oil. ¹H NMR (CDCl₃, 500 MHz) δ 3.41 (t, J=6.9 Hz, 2H), 3.25 (t, J=7.0 Hz, 2H), 1.85 (p, J=6.9 Hz, 2H), 1.60 (p, J=7.0 Hz, 2H), 1.45-1.27 (m, 16H). ¹³C NMR (CDCl₃, 150 MHz) δ 51.7, 34.2, 33.0, 29.7, 29.64, 29.60, 29.3, 29.0, 28.9, 28.4, 26.9.

### tert-Butyl (2-((12-azidododecyl)-oxy)ethyl)carbamate (Compound S18)

A mixture of NaH (60% in oil, 0.13 g, 3.9 mmol) in anhydrous DMF (3.0 mL) was treated with a solution of N-Boc-ethanolamine (0.58 g, 3.6 mmol) dissolved in anhydrous DMF (2.0 mL) at 0° C. After being stirred at 0° C. for 10 min, the reaction mixture was treated with Bu₄NI (0.55 g, 1.5 mmol) and a solution of Compound S17 (0.87 g, 3.0 mmol) in anhydrous DMF (5.0 mL). The resulting reaction mixture was warmed to rt and stirred for 24 h. The reaction was quenched with the addition of H₂O (20 mL), and the solution was acidified to pH 6-7 by the addition of saturated aqueous NH₄Cl. The mixture was extracted with EtOAc (40 mL×3), and the combined organic phase was dried over anhydrous Na₂SO₄, filtered and concentrated under reduced pressure. Flash chromatography (SiO₂, 0-20% EtOAc-hexanes gradient) afforded Compound S18 (0.64 g, 58%) as a colorless oil. ¹H NMR (CDCl₃, 600 MHz) δ 3.45 (t, J=5.2 Hz, 2H), 3.40 (t, J=6.7 Hz, 2H), 3.29 (q, J=5.3 Hz, 2H), 3.24 (t, J=7.0 Hz, 2H), 1.61-1.52 (m, 4H), 1.43 (s, 9H), 1.37-1.26 (m, 16H). ¹³C NMR (CDCl₃, 150 MHz) δ 156.2, 79.4, 71.4, 69.8, 51.7, 40.6, 29.8 (29.82 and 29.76), 29.7 (29.73 and 29.70), 29.6, 29.3, 29.0, 28.6, 26.9, 26.3. HRMS (ESI-TOF) m/z calcd for C₁₉H₃₉N₄O₃ [M+H]⁺ 371.3022, found 371.3035.

### tert-Butyl (2-((12-aminododecyl)oxy) ethyl)carbamate (Compound S19)

A reaction solution of Compound S18 (0.15 g, 0.40 mmol) in THF (3.6 mL) and H₂O (0.3 mL) was treated with polymer bound Ph₃P (0.30 g) as a solid at rt. The resulting reaction mixture was stirred at rt for overnight (ca 18 h), and the mixture was passed through a pad of Celite with CH₂Cl₂ as a rinsing solvent. The filtrate was concentrated to afford the crude residue Compound S19 (0.13 g, 94%), which was used in the next step without further purification. ¹H NMR (CDCl₃, 600 MHz) δ 3.45 (t, J=5.2 Hz, 2H), 3.40 (t, J=6.7 Hz, 2H), 3.29 (q, J=5.4 Hz, 2H), 2.67 (t, J=7.1 Hz, 2H), 1.91 (br, 2H), 1.54 (p, J=6.9 Hz, 2H), 1.45-1.41 (m, 11H), 1.32-1.25 (m, 16H) 13C NMR (CDCl₃, 150 MHz) δ156.2, 79.4, 71.4, 69.8, 42.3, 40.6, 33.7, 29.83, 29.80, 29.78, 29.72, 29.67, 28.6, 27.1, 26.3. HRMS (ESI-TOF) m/z calcd for C₁₉H₄₁N₂O₃ [M+H]⁺ 345.3117, found 345.3124.

FIG. 9 illustrates Reaction Scheme 6 that shows the reactions leading to Compound 521, whose detailed syntheses are set out below.

### tert-Butyl 2-((12-azidododecyl)oxy)acetate (Compound S20)

A mixture of NaH (60% in oil, 64 mg, 1.9 mmol) in anhydrous DMF (1.5 mL) was treated with a solution of tert-butyl 2-hydroxyacetate (0.24 g, 1.8 mmol) dissolved in anhydrous DMF (1.5 mL) at 0° C. After being stirred at 0° C. for 10 min, the reaction mixture was treated with Bu₄NI (0.28 g, 0.75 mmol) and a solution of Compound S17 (0.43 g, 1.5 mmol) in anhydrous DMF (3.0 mL). The resulting reaction mixture was warmed to rt and stirred for 24 h. The reaction was quenched with the addition of H₂O (10 mL), and the solution was acidified to pH 6-7 by the addition of saturated aqueous NH₄Cl. The mixture was extracted with EtOAc (20 mL×3), and the combined organic phase was dried over anhydrous Na₂SO₄, filtered and concentrated under reduced pressure. Flash chromatography (SiO₂, 0-20% EtOAc-hexanes gradient) afforded Compound S20 (0.14 g, 27%) as a colorless oil. ¹H NMR (CDCl₃, 500 MHz) δ 3.94 (s, 2H), 3.50 (t, J=6.7 Hz, 2H), 3.25 (t, J=7.0 Hz, 2H), 1.64-1.56 (m, 4H), 1.48 (s, 9H), 1.37-1.26 (m, 16H). ¹³C NMR (CDCl₃, 125 MHz) δ 170.1, 81.6, 72.1, 69.0, 51.7, 29.9, 29.75, 29.72, 29.68, 29.4, 29.1, 28.3, 26.9, 26.2. HRMS (ESI-TOF) m/z calcd for C₁₈H₃₅N₃O₃Na [M+Na]+364.2576, found 364.2578.

### tert-Butyl 2-((12-aminododecyl)oxy)acetate (Compound S21)

A suspension of Compound S20 (30 mg, 0.088 mmol) and 10% Pd/C (15 mg) in MeOH (1.0 mL) in a 1 dram vial was stirred at rt under 1 atm of hydrogen gas (a H₂ balloon) for overnight (ca. 18 h). The mixture was filtered through a pad of Celite. Concentration of the filtrate afforded the crude residue Compound S21 (25 mg, 90%), which was used in the next step without further purification. ¹H NMR (CDCl₃, 500 MHz) δ 3.92 (s, 2H), 3.48 (t, J=6.7 Hz, 2H), 2.81-2.73 (m, 2H), 1.66-1.56 (m, 4H), 1.46 (s, 9H), 1.32-1.23 (m, 16H). HRMS (ESI-TOF) m/z calcd for C₁₈H₃₈NO₃ [M+H]⁺ 316.2852, found 316.2851.

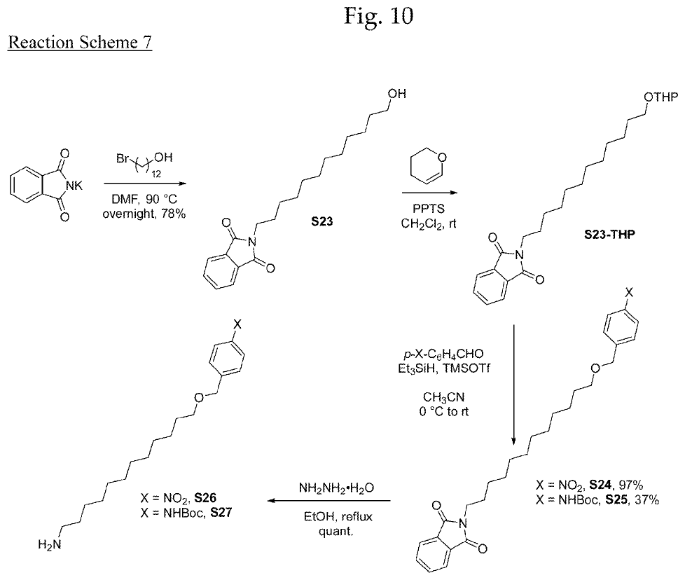

FIG. 10 illustrates Reaction Scheme 7 that shows the reactions leading to Compounds S26 and S27, whose detailed syntheses are set out below.

### 2-(12-Hydroxydodecyl) isoindoline-1,3-dione (Compound S23)

A reaction mixture of potassium phthalimide (4.1 g, 22 mmol) and 12-bromododecan-1-ol (5.3 g, 20 mmol) in anhydrous DMF (40 mL) was stirred at 90° C. for overnight (ca. 18 h) and then cooled to room temperature. The reaction was quenched with the addition of H₂O (50 mL) and the solution was extracted with EtOAc (60 mL×3). The combined organic phase was dried over anhydrous Na₂SO₄, filtered and concentrated under reduced pressure. Flash chromatography (SiO₂, 2-5% EtOAc—CH₂Cl₂ gradient) afforded Compound S23 (5.7 g, 78%) as a white solid. ¹H NMR (CDCl₃, 400 MHz) δ 7.84 (dd, J=5.4, 3.1 Hz, 2H), 7.70 (dd, J=5.5, 3.0 Hz, 2H), 3.67 (t, J=7.4 Hz, 2H), 3.64 (t, J=6.6 Hz, 2H), 1.67 (p, J=7.3 Hz, 2H), 1.59-1.52 (m, 2H), 1.34-1.25 (m, 16H). HRMS (ESI-TOF) m/z calcd for C₂₀H₃₀NO₃ [M+H]⁺ 332.2226, found 332.2229.

### 2-(12-((4-Nitrobenzyl)oxy)dodecyl)-isoindoline-1,3-dione (Compound S24)³⁷

A mixture of Compound S23-THP³⁸ (1.0 g, 2.5 mmol) and Et₃SiH (1.0 mL, 6.3 mmol) in anhydrous MeCN (1.5 mL) was added to a solution of TMSOTf (76 μL, 0.42 mmol) in MeCN (1.0 mL) followed by addition of a solution of 4-nitrobenzaldehyde (0.32 g, 2.1 mmol) in MeCN (5.0 mL) at 0° C. under Ar. The resulting mixture was stirred at rt for overnight, before the reaction was quenched with the addition of saturated aqueous NaHCO₃ (6 mL) and the solution was diluted with H₂O (15 mL). The mixture was extracted with CH₂Cl₂ (25 mL×3), and the combined organic phase was dried over anhydrous Na₂SO₄, filtered and concentrated under reduced pressure. Flash chromatography (SiO₂, 5-10% EtOAc-hexanes gradient) afforded Compound S24 (0.95 g, 97%) as a light pale solid. ¹H NMR (CDCl₃, 500 MHz) δ 8.20 (d, J=8.7 Hz, 2H), 7.84 (dd, J=5.4, 3.0 Hz, 2H), 7.70 (dd, J=5.5, 3.0 Hz, 2H), 7.50 (d, J=8.9 Hz, 2H), 4.59 (s, 2H), 3.69-3.66 (m, 2H), 3.51 (t, J=6.6 Hz, 2H), 1.69-1.61 (m, 4H), 1.38-1.25 (m, 16H). HRMS (ESI-TOF) m/z calcd for C₂₇H₃₅N₂O₅ [M+H]⁺ 467.2546, found 467.2546.

### tert-Butyl (4-(((12-(1,3-dioxoisoindolin-2-yl)-dodecyl)oxy)methyl)phenyl)carbamate (Compound S25)³⁷

A mixture of S23-THP³⁸ (1.9 g, 4.5 mmol) and Et₃SiH (1.8 mL, 11 mmol) in anhydrous MeCN (3.0 mL) was added a solution of TMSOTf (0.14 mL, 0.75 mmol) in MeCN (2.0 mL) followed by addition of a solution of tert-butyl (4-formylphenyl)carbamate (0.82 g, 3.7 mmol) in MeCN (8.0 mL) at 0° C. under Ar. The resulting mixture was stirred at rt for overnight (ca. 18 h), before the reaction was quenched with the addition of saturated aqueous NaHCO₃ (8 mL) and the solution was diluted with H₂O (20 mL). The mixture was extracted with CH₂Cl₂ (30 mL×3), and the combined organic phase was dried over anhydrous Na₂SO₄, filtered and concentrated under reduced pressure. Flash chromatography (SiO₂, 5-10% EtOAc-hexanes gradient) afforded Compound S25 (0.75 g, 37%) as a white solid. ¹H NMR (CDCl₃, 500 MHz) δ 7.84 (dd, J=5.4, 3.1 Hz, 2H), 7.70 (dd, J=5.5, 3.0 Hz, 2H), 7.33 (d, J=8.1 Hz, 2H), 7.25 (d, J=8.1 Hz, 2H), 6.5 (br, 1H), 4.43 (s, 2H), 3.67 (t, J=7.3 Hz, 2H), 3.42 (t, J=6.7 Hz, 2H), 1.66 (p, J=7.3 Hz, 2H), 1.61-1.55 (m, 2H), 1.51 (s, 9H), 1.34-1.24 (m, 16H). ¹³C NMR (CDCl₃, 125 MHz) δ 168.7, 152.9, 137.9, 134.0, 133.5, 132.4, 128.7, 123.3, 118.6, 80.6, 72.6, 70.5, 38.3, 29.9, 29.74, 29.71, 29.65, 29.63, 29.4, 28.8, 28.5, 27.1, 26.4. HRMS (ESI-TOF) m/z calcd for C₃₂H₄₄N₂O₅Na [M+Na]+559.3142, found 559.3139.

### 12-((4-Nitrobenzyl)oxy)dodecan-1-amine hydrogen chloride (Compound S26)

A solution of Compound S24 (0.10 g, 0.21 mmol) in hydrazine hydrate (1.5 mL) and EtOH (3.0 mL) was stirred at 80° C. for 2.5 h, and cooled to rt. The reaction was quenched by the dropwise addition of aqueous 1 N HCl to adjust the pH 8-9, and the solution was further diluted with H₂O. The mixture was extracted with CH₂Cl₂ (15 mL×3), and the combined organic phase was dried over anhydrous Na₂SO₄, filtered, and concentrated in vacuo to afford Compound S26 (70 mg, 99%) as a pale-yellow solid, which was used without further purification. ¹H NMR (CDCl₃, 500 MHz) δ 8.20 (d, J=8.7 Hz, 2H), 7.50 (d, J=8.7 Hz, 2H), 4.59 (s, 2H), 3.51 (t, J=6.6 Hz, 2H), 2.87-2.81 (m, 2H), 1.67-1.59 (m, 4H), 1.39-1.26 (m, 16H). HRMS (ESI-TOF) m/z calcd for C₁₉H₃₃N₂O₃ [M+H]⁺ 337.2491, found 337.2498.

### tert-Butyl (4-(((12-aminododecyl)oxy)-methyl)phenyl)carbamate (Compound S27)

A solution of Compound S25 (0.11 g, 0.20 mmol) in hydrazine hydrate (1.5 mL) and EtOH (3.0 mL) was stirred at 80° C. for 2.5 h, and cooled to rt. The reaction was quenched by the dropwise addition of aqueous 1 N HCl to adjust the pH 8-9, and the solution was further diluted with H₂O. The mixture was extracted with CH₂Cl₂ (15 mL×3), and the combined organic phase was dried over anhydrous Na₂SO₄, filtered, and concentrated in vacuo to afford Compound S27 (81 mg, 99%) as a pale-yellow solid, which was used without further purification. ¹H NMR (CDCl₃, 500 MHz) δ 7.33 (d, J=8.2 Hz, 2H), 7.25 (d, J=8.2 Hz, 2H), 4.43 (s, 2H), 3.42 (t, J=6.7 Hz, 2H), 2.72-2.70 (m, 2H), 1.61-1.55 (m, 2H), 1.53-1.46 (m, 11H), 1.34-1.25 (m, 16H). HRMS (ESI-TOF) m/z calcd for C₂₄H₄₃N₂O₃ [M+H]⁺ 407.3268, found 407.3270.

### Compound Assays

### EC₅₀ Determination:

THP-1 (American Type Culture Collection) cells, a human monocytic leukemia cell line, were differentiated by treatment with 100 nM PMA (Sigma) in Roswell Park Memorial Institute (RPMI) cell culture medium [RPMI containing 10% vol/vol FBS (Gemini Bio Products), 1% penicillin and streptomycin (Life Technologies)] for 24 h. After that, the cells were washed with PBS and cultured in fresh RPMI cell culture medium for 24 h before use in studies. PMA-differentiated THP-1 cells were seeded onto 96-well plates at 0.5×10⁵ cells per well and treated with systematically varied concentrations of the candidate compounds (dissolved in DMSO, and the final assay DMSO concentration (0.1%) was kept constant in all experiments) for 4 h. Ultra-pure LPS (dissolved in H₂O, Enzo Life Sciences) was assessed alongside the compounds as a positive control and DMSO treated control was used as a negative control. Human TNF-α in the supernatants was measured by ELISA (eBioscience). This assay was used for establishing dose-response curves and measured EC₅₀'s. EC₅₀'s were calculated by Graphpad Prism. All data is the mean of duplicates and SD is within +10% of mean.

EC₅₀ Measurement of TNF-α Release from Mouse Macrophages:

Thioglycolate-elicited macrophages were recovered 4 days after i.p. injection of 2 mL BBL thioglycolate medium, brewer modified (4%; BD Biosciences) by peritoneal lavage with 5 mL phosphate buffered saline (PBS). The peritoneal macrophages were cultured in DMEM cell culture medium [DMEM containing 10% FBS (Gemini Bio Products), 1% penicillin and streptomycin (Life Technologies)] at 37° C. and 95% air/5% CO₂. Cells were seeded onto 96-well plates at 1×10³ cells per well and treated with systematically varied concentrations of the candidate compounds (dissolved in DMSO, and final assay DMSO concentrations (0.5%) were kept constant in all experiments) for 4 h. Ultra-pure LPS (dissolved in H₂O, Enzo Life Sciences) was assessed alongside the compounds as a positive control and DMSO treated controls. 1% DMSO or less does not induce TNF-α release from mouse macrophages. It has been reported that DMSO (1%) enhances LPS induced IL-1b release, but has no effect on LPS-induced TNF-α and IL-6 release or NF-kB activation. See: Xing get al., J. Immunol. 2005, 174, 6195-6202. Mouse TNF-α in the supernatants was measured by ELISA kits according to the manufacturer's instructions (eBioscience). EC₅₀'s were calculated by Graphpad Prism. All data is mean of duplicates and SD is within ±10% of mean. Mouse cells were from wild type C57BL/6J mice.

### Immunization and In Vivo Assays of CTL Activity, Antibody Responses, and Tumor Growth

C57BL/6J mice were purchased from The Jackson Laboratory. All study procedures using mice were approved by the Institutional Animal Care and Use Committee (IACUC) of the University of Texas Southwestern Medical Center and were conducted in accordance with institutionally approved protocols and guidelines for animal care and use. All the mice were maintained at the University of Texas Southwestern Medical Center in accordance with institutionally approved protocols.

Mice were injected i.m. on day 0 and day 28 (FIG. 1) with 100 μg OVA alone, or 100 μg OVA mixed with vehicle (DMSO:Tween 80:saline=1:1:8), or mixed with 200 μg diprovocim-1 (TNF-α 1) or diprovocim-X (TNF-α 35) per mouse. EndoFit ovalbumin (OVA) with >98% purity minimum (SDS-PAGE) and endotoxin levels <1 EU/mg was purchased from Invivogen.

### CTL Assay:

On day 9 and day 37 (FIG. 1), naive C57BL/6J mice were killed, and splenocytes were collected. Half of the splenocytes were left unpulsed, and half were pulsed with OVA peptide (SIINFEKL, SEQ ID NO: 1) for 2 h in complete medium [RPMI containing 10% vol/vol FBS, 1% penicillin and streptomycin] at 37° C. The unpulsed and peptide-pulsed cells were labeled, respectively, with 0.5 pM (“low”) or 5 pM (“high”) CellTrace Violet (Invitrogen) in serum-free medium for 20 min. Equal numbers (2×10⁶) of CellTrace Violet^{high} (OVA pulsed) and CellTrace Violet^{low} (unpulsed) cells were mixed together and injected intravenously into the immunized mice.

After 48 h, blood from treated mice was collected and subjected to flow cytometry analysis. The numbers of remaining live CellTrace Violet^{high} and CellTrace Violet^{low} cells were determined and used to calculate the percentage of OVA peptide-pulsed target cells killed. Specific killing was defined as the ratio=CellTrace Violet^{low} cells/CellTrace Violet^{high} cells. The percentage of target cell lysis=[1−unimmunized ratio/immunized ratio]×100.

### Antibody Response Assay:

On day 14 (FIG. 1), serum titers of OVA-specific IgG, IgG1, or IgG2b (SouthernBiotech) were measured by ELISA. Nunc MaxiSorp flat-bottom 96-well microplates (Thermo Fisher Scientific) were coated with 5 μg/mL soluble OVA (Invivogen) and incubated at 4° C. overnight (ca. 18 h). Plates were washed four times with washing buffer (0.05% [vol/vol] Tween-20 in PBS), then blocked with 1% (vol/vol) BSA in PBS for 1 h at room temperature. Serum samples diluted in 1% (vol/vol) BSA were added to the prepared plates and incubated for 2 h. The plates were washed eight times with washing buffer and then incubated with HRP-conjugated goat anti-mouse IgG, IgG1, or IgG2b for 1 h at room temperature. Plates were washed eight times with washing buffer, then developed with SureBlue TMB Microwell Peroxidase Substrate and TMB Stop Solution (KPL). Absorbance was measured at 450 nm on a Synergy Neo2 Plate Reader (BioTek).

### Tumor Growth Assay:

B16-OVA cells (B16F10 melanoma cells stably expressing chicken ovalbumin) were grown in DMEM containing 10% vol/vol FBS. On day 38 (FIG. 1), a total of 2×10⁵ B16-OVA cells in 100 μL PBS were injected s.c. into the right flank of 17 week-old male C57BL/6J mice to establish tumors. On days 41, 44, and 47, the mice were injected i.p. with 200 μg checkpoint inhibitor (anti-mPD-L1, BioXcell) in 100 μL saline. Tumors were measured with a digital caliper (Fisher) and the tumor sizes were calculated using the following formula: volume=0.5×length×width². Mice were sacrificed when the tumor length or width reached 2 cm.

### CITATIONS

1. Hoebe et al., Nat. Immunol. 2004, 5, 971-974.
2. (a) Moresco et al., Curr. Biol. 2011, 21, R488-R493; (b) Kawai et al., Nat. Immunol. 2010, 13, 373-384.
3. (a) Kanzler et al., Nat. Med. 2007, 13, 552-559; (b) Hoebe et al., Curr. Pharmaceut. Des. 2006, 12, 4123-4134.
4. (a) Federico et al., J. Med. Chem. 2020, 63, 13466-13513; (b) Wang et al., Acc. Chem. Res. 2020, 53, 1046-1055; (c) Wang et al., Chem. Soc. Rev. 2013, 42, 4859-4866; (d) Hennessy et al., Nat. Rev. Drug Discovery 2010, 9, 293-301; and (e) Czarniecki et al., J. Med. Chem. 2008, 51, 6621-6626.
5. (a) Aldous et al., Bioorg. Med. Chem. 2018, 26, 2842-2849; (b) Sahin et al., Science 2018, 359, 1355-1360; and (c) Ribas et al., Science 2018, 359, 1350-1354.
6. (a) Metzger et al., Int. J. Peptide Protein Res. 1991, 37, 46-57; (b) Bessler et al., J. Immunol. 1985, 135, 1900-1905; and (c) Muhlradt et al., J. Exp. Med. 1997, 185, 1951-1958.
7. Deres et al., Nature 1989, 342, 561-564.
8. (a) Takeuchi et al., J. Immunol. 2002, 169, 10-14; and (b) Buwitt-Beckmann et al., J. Biol. Chem. 2006, 281, 9049-9057.
9. (a) Kang et al., Immunity 2009, 31, 873-884; (b) Jin et al., Cell 2007, 130, 1071-1081; and (c) Jin et al., Curr. Opin. Immunol. 2008, 20, 414-419.
10. (a) Lu et al., Org. Biomol. Chem. 2020, 18, 5073-5094; and (b) Kaur et al., J. Med. Chem. 2021, 64, 233-278.
11. (a) Guo et al., Front. Immunol. 2017, 8, Article 158; (b) Santone et al., Hum. Vaccin. Immunother. 2015, 11, 2038-2050; (c) Salunke et al., J. Med. Chem. 2013, 56, 5885-5900; (d) Salunke et al., J. Med. Chem. 2012, 55, 3353-3363; and (e) Agnihotri et al., J. Med. Chem. 2011, 54, 8148-8160.
12. (a) Guan et al., J. Biol. Chem. 2010, 285, 23755-23762; (b) Cheng et al., Sci. Adv. 2015, 1, e1400139; (c) Murgueitio et al., Biochim. Biophys. Acta—Gen. Subj. 2017, 1861, 2680-2681; (d) Chen et al., Chem. Commun. 2018, 54, 11411-11414; (e) Hu et al., ACS Med. Chem. Lett. 2019, 10, 132-136; and (f) Chen et al., J. Med. Chem. 2021, 64, 7371-7389.
13. (a) Goldberg et al., J. Am. Chem. Soc. 2002, 124, 544-555; (b) Boger et al., Angew. Chem. Int. Ed. 2003, 42, 4138-4176; and (c) Whitby et al., Acc. Chem. Res. 2012, 45, 1698-1709.
14. Wang et al., Proc. Natl. Acad. Sci. USA 2018, 115, E8698-E8706; and U.S. Pat. No. 11,040,959 Jun. 22, 2021.
15. Morin et al., J. Am. Chem. Soc. 2018, 140, 14440-14454.
16. Su et al., J. Med. Chem. 2019, 62, 2938-2949.
17. Ignacio et al., Bioconjugate Chem. 2018, 29, 587-603.
18. It is noteworthy that the characteristics of such an immune stimulator are different from those of a conventional drug.¹⁷ It is administered infrequently as part of prime and boost vaccinations, it is typically administered i.m.; the vaccination not only providing long term systemic protection but does so with a localized low dose administration, and the acceptable chemical (e.g.; Mwt), physical (e.g.; low solubility and high c Log P), PK (e.g.; short vs long circulating half-life in blood), permeability (low vs high), and metabolic stability properties that are distinct from those of a traditional drug.¹⁷
19. (a) The experimental procedure followed the method reported in Raffi et al., Synthesis 2007, 3013-3016, using 12-aminododecanoic acid (500 mg, 2.32 mmol), AcCl (3.3 mL, 46.4 mmol), and MeOH (10 mL) to afford methyl 12-aminododecanoate hydrochloride (0.60 g, 97%); (b) The spectroscopic data matched those found in the previous report. Jain et al., Bioconjugate Chem. 2010, 21, 2110-2118.
20. The experimental procedure followed the method reported in citation 19a, using 8-aminododecanoic acid (0.13 g, 0.80 mmol), AcCl (1.1 mL, 16 mmol), and MeOH (3.5 mL) to afford methyl 8-aminododecanoate hydrochloride (0.17 g, 99%). The spectroscopic data matched those from a previous report: Lintnerova et al., Monatsh. Chem. 2018, 149, 901-911.
21. The experimental procedure followed the method reported in citation 19a, using 6-aminododecanoic acid (0.13 g, 1.0 mmol), AcCl (1.4 mL, 20 mmol), and MeOH (4.5 mL) to afford methyl 6-aminodo decanoate hydrochloride (0.18 g, 99%). The spectroscopic data matched those from the previous report. Jakobsen et al., PCT WO 2001007912, 2001.
22. Battah et al., J. Med. Chem. 2017, 60, 3498-3510.
23. A synthesis of this starting material is provided above.
24. The experimental procedure for the preparation of this methyl ester followed the method reported in citation 19a, using 3-(2-(2-(2-aminoethoxy)-ethoxy)ethoxy)propanoic acid (55 mg, 0.25 mmol), AcCl (0.35 mL, 5.0 mmol), and MeOH (1.2 mL).
25. Glansdorp et al., Org. Biomol. Chem. 2008, 6, 4120-4124.
26. Naturale e al., Eur. J. Org. Chem. 2012, 5774-5788.
27. Yan et al., ACS Sens. 2018, 3, 2621-2628.
28. (a) Zhou et al., Chem. Commun. 2018, 54, 6036-6039; and (b) Yang e al., J. Am. Chem. Soc. 2010, 132, 656-666.
29. Morimoto et al., Biomacromolecules 2013, 14, 56-63.
30. Kuehl et al., PCT WO 2003064360, 2003.
31. Rawling et al., PCT WO 2015176135, 2015.
32. Fujimoto et al., Org. Lett. 2013, 15, 184-187.
33. Iwan et al., RSC Adv. 2015, 5, 70127-70138.
34. Bigi et al., J. Am. Chem. Soc. 2013, 135, 7831-7834.
35. The preparation followed the procedure of the previous report: Wang et al., Adv. Synth. Catal. 2009, 351, 415-422.
36. The preparation of compounds S24 and S25 followed a reported procedure: Suzuki et al., Synthesis 1999, 1561-1563.
37. Compound S23-THP was prepared by following a reported protocol using Compound S23 (0.91 g, 2.5 mmol), 3,4-dihydro-2H-pyran (0.34 mL, 3.7 mmol), PPTS (0.13 g, 0.5 mmol), and anhydrous CH₂Cl₂ (12 mL). Miyashita et al., J. Org. Chem. 1977, 42, 3772-3774.

Each of the patents, patent applications and articles cited herein is incorporated by reference.

The foregoing description and the examples are intended as illustrative and are not to be taken as limiting. Still other variations within the spirit and scope of this invention are possible and will readily present themselves to those skilled in the art.

1. A compound of Formula I, wherein 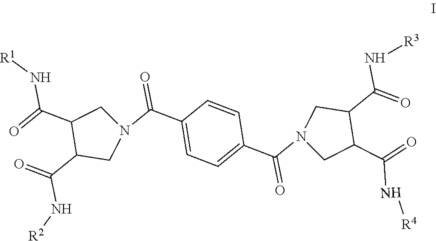
R¹, R² and R³ are the same or different and are a trans-2-phenylcyclopropyl, or a trans-2-(4-fluorophenyl)cyclopropyl group, and
R⁴ is a composite of (a) a hydrocarbyl group bonded to the depicted amido nitrogen atom and (b) a substituent group bonded thereto as discussed below, the hydrocarbyl group has a longest chain of atoms that has a total length that is about that of a saturated chain of about 2 carbon atoms (an ethylene group), and a length that is less than that of a saturated chain of about 20 carbon atoms [an eicosylene group],
said hydrocarbyl group including a methylene group (—CH₂—) that is 1) bonded directly to or 2) bonded indirectly and distal to the amido nitrogen of the depicted —C(O)NH—R⁴ group,
and said methylene group is also bonded to a substituent group that is one or another of i) a phenyl group that includes a substituent selected from the group consisting of a hydroxyl, amino, carboxy, C₁-C₆ alkyl carboxylate, sulfo, C₁-C₆ alkyl sulfonate, fluoro, azido and an ethynyl group, ii) a hydroxyl, a mercaptan, amine, mono- or disubstituted amine, an azido group or iii) to an oxygen, nitrogen or sulfur atom that is part of a substituent group that is selected from the group consisting of an amino acid, ether, ester, carbonate, carbamate, thioether, thioester, thiourea, amido, mono- or disubstituted amide, urea, and a N′-mono or N′,N′-disubstituted urea, wherein said substituent group contains a chain of atoms that include zero to four oxygens, zero to two sulfur atoms, and zero to four nitrogen atoms, with the proviso that the sum of the sulfur, nitrogen and oxygen atoms in the substituent is at least one, and not greater than eight, and
the length of said hydrocarbyl group together with the substituent bonded to said methylene of that hydrocarbyl group is less than that of a saturated chain of about 24 carbon atoms [a tetracosylene group].

2. The compound according to claim 1, wherein each depicted pyrrolidinyldicarboxamido group has the (S,S) configuration.

3. The compound according to claim 2, wherein bonds to the cyclopropyl moiety have a (1S,2R) configuration.

4. The compound according to claim 2, wherein and said compound has the structural Formula Ia, and wherein the R¹⁻⁴ moieties are as described above

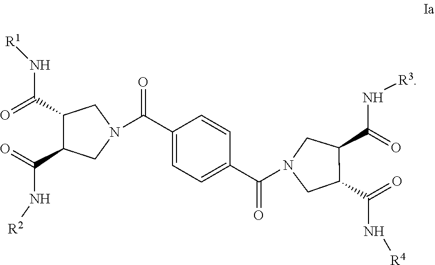

5. The compound according to claim 4 that is a single enantiomer.

6. A pharmaceutical composition comprising a concentration of a compound of claim 1 effective to induce release of TNF-α from one or both of in vitro cultured human PMA differentiated THP-1 cells and or mouse macrophages, said compound being dissolved or dispersed in a physiologically tolerable diluent.

7. The pharmaceutical composition according to claim 6, wherein said compound is a single enantiomer.

8. The compound according to claim 1, wherein said hydrocarbyl group of said composite R⁴ has a saturated chain length of about 6 to about 16 carbon atoms.

9. The compound according to claim 1, wherein said substituent group bonded to said R⁴ hydrocarbyl group is bonded to a methylene group that is bonded indirectly and distal to the amido nitrogen of the depicted —C(O)NH—R⁴ group.

10. The compound according to claim 8, wherein said methylene group is also bonded to a phenyl group that includes a substituent selected from the group consisting of a hydroxyl, amino, carboxy, C₁-C₆ alkyl carboxylate, sulfo, C₁-C₆ alkyl sulfonate, fluoro, azido and an ethynyl group.

11. The compound according to claim 8, wherein said methylene group is also bonded to a phenyl group that includes a substituent selected from the group consisting of a hydroxyl, a mercaptan, amine, mono- or disubstituted amine, and an azido group.

12. The compound according to claim 8, wherein said methylene group is also bonded to a to an oxygen, nitrogen or sulfur atom that is part of a substituent group that is selected from the group consisting of an ether, ester, carbonate, carbamate, thioether, thioester, thiourea, amido, mono- or disubstituted amide, urea, and a N′-mono or N′,N′-disubstituted urea, wherein said substituent group contains a chain of atoms that include zero to four oxygens, zero to two sulfur atoms, and zero to four nitrogen atoms, with the proviso that the sum of the sulfur, nitrogen and oxygen atoms in the substituent is at least one, and not greater than eight.

13. The compound according to claim 8, wherein said methylene group is also bonded to an oxygen atom of a carboxylic acid ester.

14. The compound according to claim 13, wherein said carboxylic acid ester is a glycine ester.

15. The compound according to claim 14, wherein said glycine ester is a Boc-protected glycine ester.

16. The compound according to claim 15, wherein said hydrocarbyl group of said composite R⁴ has a saturated chain length of about 10 to about 14 carbon atoms.

17. A method of enhancing an immunogen-specific IgG humoral immune response titer that comprises contacting immune cells with composition containing an adjuvant-effective amount of a compound of claim 1 and an immunogen to which said response is to be enhanced that are dissolved or dispersed in a physiologically tolerable diluent, wherein the enhancement in IgG titer is about 2 to about 4 times the titer observed due to the same amount of immunogen dissolved or dispersed said a physiologically tolerable diluent in the absence of said compound, and said titers are measured 14 days after said immune cell contact.

18. The method according to claim 17, wherein said cells are contacted in vivo.

19. The method according to claim 18, wherein said compound is a single enantiomer.


SEQUENCE LISTING (1 sequence)

SEQ ID NO: 1 - protein, 8 aa, Gallus gallus
  REGION 1..8: note = misc_feature - OVA peptide
SIINFEKL